SEQUENCE LISTING (72 sequences)

SEQ ID NO: 1 - protein, 819 aa, Homo Sapiens
SENGSEVAAQAGLSGPAEVPGAVGERTPRKKEPPRASPPGGLAEPPGSAGPQAGPTVVPGSATPMETGAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 2 - DNA, 2559 bp, Homo Sapiens
atgttatctgggaagaaggcggcagccgcggcggcggcggctgcagcggcagcaaccgggacggaggctggccctgggacagcaggcggctccgagaacgggtctgaggtggccgcgcagcccgcgggcctgtcgggcccagccgaggtcgggccgggggcggtgggggagcgcacaccccgcaagaaagagcctccgcgggcctcgccccccgggggcctggcggaaccgccggggtccgcagggcctcaggccggccctactgtcgtgcctgggtctgcgacccccatggaaactggaatagcagagactccggaggggcgtcggaccagccggcgcaagcgggcgaaggtagagtacagagagatggatgaaagcttggccaacctctcagaagatgagtattattcagaagaagagagaaatgccaaagcagagaaggaaaagaagcttcccccaccaccccctcaagccccacctgaggaagaaaatgaaagtgagcctgaagaaccatcgggtgtggagggcgcagctttccagagccgacttcctcatgaccggatgacttctcaagaagcagcctgttttccagatattatcagtggaccacaacagacccagaaggtttttcttttcattagaaaccgcacactgcagttgtggttggataatccaaagattcagctgacatttgaggctactctccaacaattagaagcaccttataacagtgatactgtgcttgtccaccgagttcacagttatttagagcgtcatggtcttatcaacttcggcatctataagaggataaaacccctaccaactaaaaagacaggaaaggtaattattataggctctggggtctcaggcttggcagcagctcgacagttacaaagttttggaatggatgtcacacttttggaagccagggatcgtgtgggtggacgagttgccacatttcgcaaaggaaactatgtagctgatcttggagccatggtggtaacaggtcttggagggaatcctatggctgtggtcagcaaacaagtaaatatggaactggccaagatcaagcaaaaatgcccactttatgaagccaacggacaagctgttcctaaagagaaagatgaaatggtagagcaagagtttaaccggttgctagaagctacatcttaccttagtcatcaactagacttcaatgtcctcaataataagcctgtgtcccttggccaggcattggaagttgtcattcagttacaagagaagcatgtcaaagatgagcagattgaacattggaagaagatagtgaaaactcaggaagaattgaaagaacttcttaataagatggtaaatttgaaagagaaaattaaagaactccatcagcaatacaaagaagcatctgaagtaaagccacccagagatattactgccgagttcttagtgaaaagcaaacacagggatctgaccgccctatgcaaggaatatgatgaattagctgaaacacaaggaaagctagaagaaaaacttcaggagttggaagcgaatcccccaagtgatgtatatctctcatcaagagacagacaaatacttgattggcattttgcaaatcttgaatttgctaatgccacacctctctcaactctctcccttaagcactgggatcaggatgatgactttgagttcactggcagccacctgacagtaaggaatggctactcgtgtgtgcctgtggctttagcagaaggcctagacattaaactgaatacagcagtgcgacaggttcgctacacggcttcaggatgtgaagtgatagctgtgaatacccgctccacgagtcaaacctttatttataaatgcgacgcagttctctgtacccttcccctgggtgtgctgaagcagcagccaccagccgttcagtttgtgccacctctccctgagtggaaaacatctgcagtccaaaggatgggatttggcaaccttaacaaggtggtgttgtgttttgatcgggtgttctgggatccaagtgtcaatttgttcgggcatgttggcagtacgactgccagcaggggtgagctcttcctcttctggaacctctataaagctccaatactgttggcactagtggcaggagaagctgctggtatcatggaaaacataagtgacgatgtgattgttggccgatgcctggccattctcaaagggatttttggtagcagtgcagtacctcagcccaaagaaactgtggtgtctcgttggcgtgctgatccctgggctcggggctcttattcctatgttgctgcaggatcatctggaaatgactatgatttaatggctcagccaatcactcctggcccctcgattccaggtgccccacagccgattccacgactcttctttgcgggagaacatacgatccgtaactacccagccacagtgcatggtgctctgctgagtgggctgcgagaagcgggaagaattgcagaccagtttttgggggccatgtatacgctgcctcgccaggccacaccaggtgttcctgcacagcagtccccaagcatgtga

SEQ ID NO: 3 - protein, 876 aa, Homo Sapiens
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGQAGGLQDDSSGGYGDGQASGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQADTVKVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 4 - DNA, 2631 bp, Homo Sapiens
atgttatctgggaagaaggcggcagccgcggcggcggcggctgcagcggcagcaaccgggacggaggctggccctgggacagcaggcggctccgagaacgggtctgaggtggccgcgcagcccgcgggcctgtcgggcccagccgaggtcgggccgggggcggtgggggagcgcacaccccgcaagaaagagcctccgcgggcctcgccccccgggggcctggcggaaccgccggggtccgcagggcctcaggccggccctactgtcgtgcctgggtctgcgacccccatggaaactggaatagcagagactccggaggggcgtcggaccagccggcgcaagcgggcgaaggtagagtacagagagatggatgaaagcttggccaacctctcagaagatgagtattattcagaagaagagagaaatgccaaagcagagaaggaaaagaagcttcccccaccaccccctcaagccccacctgaggaagaaaatgaaagtgagcctgaagaaccatcggggcaagcaggaggacttcaagacgacagttctggagggtatggagacggccaagcatcaggtgtggagggcgcagctttccagagccgacttcctcatgaccggatgacttctcaagaagcagcctgttttccagatattatcagtggaccacaacagacccagaaggtttttcttttcattagaaaccgcacactgcagttgtggttggataatccaaagattcagctgacatttgaggctactctccaacaattagaagcaccttataacagtgatactgtgcttgtccaccgagttcacagttatttagagcgtcatggtcttatcaacttcggcatctataagaggataaaacccctaccaactaaaaagacaggaaaggtaattattataggctctggggtctcaggcttggcagcagctcgacagttacaaagttttggaatggatgtcacacttttggaagccagggatcgtgtgggtggacgagttgccacatttcgcaaaggaaactatgtagctgatcttggagccatggtggtaacaggtcttggagggaatcctatggctgtggtcagcaaacaagtaaatatggaactggccaagatcaagcaaaaatgcccactttatgaagccaacggacaagctgacactgtcaaggttcctaaagagaaagatgaaatggtagagcaagagtttaaccggttgctagaagctacatcttaccttagtcatcaactagacttcaatgtcctcaataataagcctgtgtcccttggccaggcattggaagttgtcattcagttacaagagaagcatgtcaaagatgagcagattgaacattggaagaagatagtgaaaactcaggaagaattgaaagaacttcttaataagatggtaaatttgaaagagaaaattaaagaactccatcagcaatacaaagaagcatctgaagtaaagccacccagagatattactgccgagttcttagtgaaaagcaaacacagggatctgaccgccctatgcaaggaatatgatgaattagctgaaacacaaggaaagctagaagaaaaacttcaggagttggaagcgaatcccccaagtgatgtatatctctcatcaagagacagacaaatacttgattggcattttgcaaatcttgaatttgctaatgccacacctctctcaactctctcccttaagcactgggatcaggatgatgactttgagttcactggcagccacctgacagtaaggaatggctactcgtgtgtgcctgtggctttagcagaaggcctagacattaaactgaatacagcagtgcgacaggttcgctacacggcttcaggatgtgaagtgatagctgtgaatacccgctccacgagtcaaacctttatttataaatgcgacgcagttctctgtacccttcccctgggtgtgctgaagcagcagccaccagccgttcagtttgtgccacctctccctgagtggaaaacatctgcagtccaaaggatgggatttggcaaccttaacaaggtggtgttgtgttttgatcgggtgttctgggatccaagtgtcaatttgttcgggcatgttggcagtacgactgccagcaggggtgagctcttcctcttctggaacctctataaagctccaatactgttggcactagtggcaggagaagctgctggtatcatggaaaacataagtgacgatgtgattgttggccgatgcctggccattctcaaagggatttttggtagcagtgcagtacctcagcccaaagaaactgtggtgtctcgttggcgtgctgatccctgggctcggggctcttattcctatgttgctgcaggatcatctggaaatgactatgatttaatggctcagccaatcactcctggcccctcgattccaggtgccccacagccgattccacgactcttctttgcgggagaacatacgatccgtaactacccagccacagtgcatggtgctctgctgagtgggctgcgagaagcgggaagaattgcagaccagtttttgggggccatgtatacgctgcctcgccaggccacaccaggtgttcctgcacagcagtccccaagcatgtga

SEQ ID NO: 5 - protein, 101 aa, Artificial Sequence
  Synthetic polypeptide
MEPVDPRLEPWKHPGSQPKTACTNCYCKKCCFHCQVCFITKALGISYGRKKRRQRRRPPQGSQTHQVSLSKQPTSQSRGDPTGPKESKKKVERETETDPFD

SEQ ID NO: 6 - protein, 11 aa, Artificial Sequence
  Synthetic polypeptide
SYGRKKMRRQR

SEQ ID NO: 7 - DNA, 306 bp, Human immunodeficiency virus
atggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcaacccacctcccaatcccgaggggacccgacaggcccgaaggaatcgaagaagaaggtggagagagagacagagacagatccattcgattag

SEQ ID NO: 8 - protein, 482 aa, Homo Sapiens
MVEKGPEVSGKRRGRNNAAASASAAAASAAASAACASPAATAASGAAASSASAAAASAAAAPNNGQNKSLAAAAPNGNSSSNSWEEGSSGSSSDEEHGGGGMRVGPQYQAVVPDFDPAKLARRSQERDNLGMLVWSPNQNLSEAKLDEYIAIAKEKHGYNMEQALGMLFWHKHNIEKSLADLPNFTPFPDEWTVEDKVLFEQAFSFHGKTFHRIQQMLPDKSIASLVKFYYSWKKTRTKTSVMDRHARKQKREREESEDELEEANGNNPIDIEVDQNKESKKEVPPTETVPQVKKEKHSTQAKNRAKRKPPKGMFLSQEDVEAVSANATAATTVLRQLDMELVSVKRQIQNIKQTNSALKEKLDGGIEPYRLPEVIQKCNARWTTEEQLLAVQAIRKYGRDFQAISDVIGNKSVVQVKNFFVNYRRRFNIDEVLQEWEAEHGKEETNGPSNQKPVKSPDNSIKMPEEEDEAPVLDVRYASAS

SEQ ID NO: 9 - DNA, 1449 bp, Homo Sapiens
atggtggagaagggccccgaggtctcagggaagcggagagggaggaacaacgcggccgcctccgcctccgccgccgccgcctccgccgccgcctcggccgcctgcgcctcgccagccgccactgccgcctcgggcgccgccgcctcctcagcctcggccgccgccgcctcagccgccgccgcccccaataatggccagaataaaagtttggcggcggcggcgcccaatggcaacagcagcagcaactcctgggaggaaggcagctcgggctcgtccagcgacgaggagcacggtggcggtggcatgagggtcggaccccagtaccaggcggtggtgcccgacttcgaccccgccaaactggcaagacgcagtcaagaacgggacaatcttggcatgttggtctggtcacccaatcaaaatctgtcagaagcaaagttggatgaatacattgccattgccaaagaaaagcatgggtacaacatggaacaggctcttgggatgctcttctggcataaacataatatcgaaaagtcattggctgatttgcccaactttacccctttcccagatgagtggactgtggaagataaagtcttatttgagcaagcctttagttttcatgggaaaacttttcatagaatccaacaaatgcttccagataaatctatagcaagtctggtgaaattttactattcttggaagaagacgaggactaaaactagtgtgatggatcgccatgcccggaaacaaaaacgggagcgggaggagagcgaggatgaactggaagaggcaaatggaaacaatcccattgacattgaggttgatcaaaacaaggaaagcaaaaaggaggttccccctactgagacagttcctcaggtcaaaaaagaaaaacatagcacacaagctaaaaatagagcaaaaaggaaacctccaaaaggaatgtttctttctcaagaagatgtggaggctgtttctgccaatgccactgctgctaccacggtgctgagacaactagacatggaattggtttcagtcaaacgacagatccagaatattaaacagacaaacagtgctctcaaagaaaaacttgatggtggaatagaaccatatcgacttccagaggtcattcagaaatgtaatgcacgttggactacagaagagcagcttctcgccgtacaagccatcaggaaatatggccgagattttcaggcaatctcagacgtgattgggaacaaatcagtggtacaagtgaaaaacttttttgtaaattatcgacgccgcttcaacatagatgaagttttacaagaatgggaggcagaacatggtaaagaagagaccaatgggcccagtaaccagaagcctgtgaagtccccagataattccattaagatgcccgaagaggaagacgaggctcctgttctggatgtcagatatgcatctgcctcctga

SEQ ID NO: 10 - protein, 634 aa, Homo Sapiens
MELQTLQEALKVEIQVHQKLVAQMKQDPQNADLKKQLHELQAKITALSEKQKRVVEQLRKNLIVKQEQPDKFQIQPLPQSENKLQTAQQQPLQQLQQQQQYHHHHAQQSAAASPNLTASQKTVTTASMITTKTLPLVLKAATATMPASVVGQRPTIAMVTAINSQKAVLSTDVQNTPVNLQTSSKVTGPGAEAVQIVAKNTVTLQVQATPPQPIKVPQFIPPPRLTPRPNFLPQVRPKPVAQNNIPIAPAPPPMLAAPQLIQRPVMLTKFTPTTLPTSQNSIHPVRVVNGQTATIAKTFPMAQLTSIVIATPGTRLAGPQTVQLSKPSLEKQTVKSHTETDEKQTESRTITPPAAPKPKREENPQKLAFMVSLGLVTHDHLEEIQSKRQERKRRTTANPVYSGAVFEPERKKSAVTYLNSTMHPGTRKRANEEHWPKGDIHEDFCSVCRKSGQLLMCDTCSRVYHLDCLDPPLKTIPKGMWICPRCQDQMLKKEEAIPWPGTLAIVHSYIAYKAAKEEEKQKLLKWSSDLKQEREQLEQKVKQLSNSISKCMEMKNTILARQKEMHSSLEKVKQLIRLIHGIDLSKPVDSEATVGAISNGPDCTPPANAATSTPAPSPSSQSCTANCNQGEETK

SEQ ID NO: 11 - DNA, 1905 bp, Homo Sapiens
atggagttgcagactctacaggaggctcttaaagtggaaattcaggttcaccagaaactggttgctcaaatgaagcaggatccacagaatgctgacttaaagaaacagcttcatgaactccaagccaaaatcacagctttgagtgagaaacagaaaagagtagttgaacagctacggaagaacctgatagtaaagcaagaacaaccggacaagttccaaatacagccattgccacaatctgaaaacaaactacaaacagcacagcagcaaccactacagcaactacaacaacagcagcagtaccaccaccaccacgcccagcagtcagctgcagcctctcccaacctgactgcttcacagaagactgtaactacagcttctatgattaccacaaagacactacctctcgtcttgaaagcagcaactgcgaccatgcctgcctctgtggtgggccagagacctaccattgctatggtgaccgccatcaacagtcagaaggctgtgctcagcactgatgtgcagaacacaccagtcaacctccagacgtctagtaaggtcactgggcctggggcagaggctgtccaaattgtggcaaaaaacacagtcactctgcaggttcaggcaacacctcctcagcccatcaaagtaccacagtttatcccccctcctagactcactccacgtccaaactttcttccacaggttcgacccaagcctgtggcccagaataacattcctattgccccagcaccacctcccatgctcgcagctcctcagcttatccagaggcccgtcatgctgaccaagttcacccccacaacccttcccacatcccagaattccatccaccccgtccgtgtcgtcaatgggcagactgcaaccatagccaaaacgttccccatggcccagctcaccagcattgtgatagctactccagggaccagactcgctggacctcaaactgtacagcttagcaagccaagtcttgaaaaacagacagttaaatctcacacagaaacagatgagaaacaaacagagagccgcaccatcaccccacctgctgcacccaaaccaaaacgggaggagaaccctcagaaacttgccttcatggtgtctctagggttggtaacacatgaccatctagaagaaatccaaagcaagaggcaagagcgaaaaagaagaacaacagcaaatccggtctacagtggagcagtctttgagccagagcgtaagaagagtgcagtgacatacctaaacagcacaatgcaccctgggacccggaagagagccaatgaggaacactggccaaagggtgatattcatgaggatttttgcagcgtttgcagaaaaagtggccagttactgatgtgcgacacatgttcccgtgtatatcatttggactgcttagacccccctctgaaaacaattcccaagggcatgtggatctgtcccagatgtcaggaccagatgctgaagaaggaagaagcaattccatggcctggaactttagcaattgttcattcctatattgcctacaaagcagcaaaagaagaagagaaacagaagttacttaaatggagttcagatttaaaacaagaacgagaacaactagagcaaaaggtgaaacagctcagcaattccataagtaaatgcatggaaatgaagaacaccatcctggcccggcagaaggagatgcacagctccctggagaaggtaaaacagctgattcgcctcatccacggcatcgacctctccaaacctgtagactctgaggccactgtgggggccatctccaatggcccggactgcaccccccctgccaatgccgccacctccacgccggccccttccccctcctcccagagctgcacagcgaactgtaaccagggggaagagactaaataa

SEQ ID NO: 12 - protein, 101 aa, Human immunodeficiency virus; identical to SEQ ID NO: 5

SEQ ID NO: 13 - protein, 86 aa, Human immunodeficiency virus
MEPVDPRLEPWKHPGSQPKTACTNCYCKKCCFHCQVCFITKALGISYGRKKRRQRRRPPQGSQTHQVSLSKQPTSQSRGDPTGPKE

SEQ ID NO: 14 - protein, 86 aa, Human immunodeficiency virus
MEPVDPRLEPWKHPGSQPKTACTNCYCKKCCFHCQVCFITKALGISYGRKKRRQRRRAPQGSQTHQVSLSKQPTSQSRGDPTGPKE

SEQ ID NO: 15 - protein, 86 aa, Human immunodeficiency virus
MEPVDPRLEPWKHPGSQPKTACTNCYCKKCCFHCQVCFITKALGISYGRKKRRQRRRPPQGSQTHQVSLSKQPTSQPRGDPTGPKE

SEQ ID NO: 16 - protein, 86 aa, Human immunodeficiency virus
MEPVDPRLEPWKHPGSQPKTACTTCYCKKCCFHCQVCFTTKALGISYGRKKRRQRRRPPQGSQTHQVSLSKQPTSQPRGDPTGPKE

SEQ ID NO: 17 - protein, 101 aa, Human immunodeficiency virus
MEPVDPRLEPWKHPGSQPKTACNNCYCKKCCYHCQVCFLTKGLGISYGRKKRRQRRGPPQGSQTHQVSLSKQPTSQPRGDPTPKESKEKVERETETDPAVQ

SEQ ID NO: 18 - protein, 86 aa, Human immunodeficiency virus
MEPVDPRLEPWKHPGSQPKTACTNCYCKKCCFHCQVCFITKALGISYGRKKRRQRRRAHQNSQTHQASLSKQPTSQPRGDPTGPKE

SEQ ID NO: 19 - protein, 101 aa, Human immunodeficiency virus
MEPVDPRLEPWKHPGSQPKTACTNCYCKKCCFHCQVCFTKKALGISYGRKKRRQRRRAHQDSQNHQASLSKQPSSQTRGDPTGPKEPKKEVEREAETDPLD

SEQ ID NO: 20 - protein, 101 aa, Human immunodeficiency virus
MEPVDPNLEPWKHPGSQPRTACTNCYCKKCCFHCQVCFITKGLGISYGRKKRRQRQRAPDSSQNHQDSLSKQPSSQPRGDPTGPKESKKEVERETETDPLD

SEQ ID NO: 21 - protein, 86 aa, Human immunodeficiency virus
MDPVDPNLEPWNHPGSQPKTACNRCHCKKCCYHCQVCFITKGLGISYGRKKRRQRRRPSQGGQTHQDPIPKQPSSQPRGNPTGPKE

SEQ ID NO: 22 - protein, 86 aa, Human immunodeficiency virus
MDPVDPNIEPWNHPGSQPKTACNRCHCKKCCYHCQVCFITKGLGISYGRKKRRQRRRPSQGGQTHQDPIPKQPSSQPRGDPTGPKE

SEQ ID NO: 23 - protein, 99 aa, Human immunodeficiency virus
MDPVDPNLEPWNHPGSQPRTPCNKCHCKKCCYHCPVCFLNKGLGISYGRKKRRQRRGPPQGGQAHQVPIPKQPSSQPRGDPTGPKEQKKKVESEAETDP

SEQ ID NO: 24 - protein, 86 aa, Human immunodeficiency virus
MDPVDPNLESWNHPGSQPRTACNKCHCKKCCYHCQVCFITKGLGISYGRKKRRQRRKPPQGDQAHQVPIPEQPSSQSRGDPTGPKK

SEQ ID NO: 25 - protein, 87 aa, Human immunodeficiency virus
MDPVDPNLEPWNHPGSQPRTPCNKCYCKKCCYHCQMCFITKGLGISYGRKKRRQRRRPPQGNQAHQDPLPEQPSSQHRGDHPTGPKE

SEQ ID NO: 26 - protein, 101 aa, Human immunodeficiency virus
MEPVDPNLEPWKHPGSQPTTACSNCYCKVCCWHCQLCFLKKGLGISYGKKKRKPRRGPPQGSKDHQTLIPKQPLPQSQRVSAGQEESKKKVESKAKTDRFA

SEQ ID NO: 27 - protein, 101 aa, Human immunodeficiency virus
MEPVDPNLEPWKHPGSQPRTACNNCYCKKCCFHCYACFTRKGLGISYGRKKRRQRRRAPQDSQTHQASLSKQPASQSRGDPTGPTESKKKVERETETDPFD

SEQ ID NO: 28 - protein, 101 aa, Human immunodeficiency virus
MEPVDPNLEPWKHPGSQPRTACNNCYCKKCCFHCQVCFTKKGLGISYGRKKRRQRRRPPQDSQTHQSSLSKQPTSQLRGDPTGPTESKKKVERETETDPVH

SEQ ID NO: 29 - protein, 101 aa, Human immunodeficiency virus
MEPVDPRLEPWKHPGSQPKTASNNCYCKRCCLHCQVCFTKKGLGISYGRKKRRQRRRAPQDSKTHQVSLSKQPASQPRGDPTGPKESKKKVERETETDPED

SEQ ID NO: 30 - protein, 58 aa, Human immunodeficiency virus
MEPVDPRLEPWKHPGSQPKTACTTCYCKKCCFHCQVCFITKALGISYGRKKRRQRRRA

SEQ ID NO: 31 - protein, 101 aa, Human immunodeficiency virus
MEPVDPRLEPWKHPGSQPKTACTTCYCKKCCFHCQVCFTKKALGISYGRKKRRQRRRAPEDSQTHQVSLPKQPAPQFRGDPTGPKESKKKVERETETHPVD

SEQ ID NO: 32 - protein, 101 aa, Human immunodeficiency virus
MDPVDPRLEPWKHPGSQPKAACTSCYCKKCCFHCQVCFTTKGLGISYGRKKRRQRRRAPQDSQTHQVSLPKQPASQARGDPTGPKESKKKVERETETDPVD

SEQ ID NO: 33 - protein, 101 aa, Human immunodeficiency virus
MEPVDPRLEPWKHPGSQPKTACTNCYCKKCCFHCQVCFITKGLGISYGRKKRRQRRRAPPDSEVHQVSLPKQPASQPQGDPTGPKESKKKVERETETDPVH

SEQ ID NO: 34 - protein, 101 aa, Human immunodeficiency virus
MEPVDPSLEPWKHPGSQPKTACTNCYCKKCCLHCQVCFTTKGLGISYGRKKRRQRRRPPQDSQTHQVSLPKQPSSQQRGDPTGPKESKKKVERETETDPDN

SEQ ID NO: 35 - protein, 10 aa, Artificial Sequence
  Synthetic polypeptide
RKKMRRQRRR

SEQ ID NO: 36 - DNA, 19 bp, Artificial Sequence
  Synthetic oligonucleotide
gaaggctcttctagcaata

SEQ ID NO: 37 - protein, 88 aa, Artificial Sequence
  Synthetic polypeptide
VKKEKHSTQAKNRAKRKPPKGMFLSQEDVEAVSANATATTVLRQLDMELVSVKRQIQNIKQTNSALKEKLDGGIEPYRLPEVIQKCNA

SEQ ID NO: 38 - protein, 30 aa, Artificial Sequence
  Synthetic polypeptide
QAKNRAKRKPPKGMFLSQEDVEAVSANATA

SEQ ID NO: 39 - protein, 29 aa, Artificial Sequence
  Synthetic polypeptide
TTVLRQLDMELVSVKRQIQNIKQTNSALK

SEQ ID NO: 40 - protein, 30 aa, Artificial Sequence
  Synthetic polypeptide
ELVSVKRQIQNIKQTNSALKEKLDGGIEPY

SEQ ID NO: 41 - protein, 30 aa, Artificial Sequence
  Synthetic polypeptide
NIKQTNSALKEKLDGGIEPYRLPEVIQKCN

SEQ ID NO: 42 - protein, 28 aa, Artificial Sequence
  Synthetic polypeptide
VKKEKHSTQAKNRAKRKPPKGMFLSQED

SEQ ID NO: 43 - protein, 69 aa, Artificial Sequence
  Synthetic polypeptide
QAKNRAKRKPPKGMFLSQEDVEAVSANATATTVLRQLDMELVSVKRQIQNIKQTNSALKEKLDGGIEPY

SEQ ID NO: 44 - protein, 69 aa, Artificial Sequence
  Synthetic polypeptide
PKGMFLSQEDVEAVSANATATTVLRQLDMELVSVKRQIQNIKQTNSALKEKLDGGIEPYRLPEVIQKCN

SEQ ID NO: 45 - protein, 59 aa, Artificial Sequence
  Synthetic polypeptide
PKGMFLSQEDVEAVSANATATTVLRQLDMELVSVKRQIQNIKQTNSALKEKLDGGIEPY

SEQ ID NO: 46 - protein, 49 aa, Artificial Sequence
  Synthetic polypeptide
VEAVSANATATTVLRQLDMELVSVKRQIQNIKQTNSALKEKLDGGIEPY

SEQ ID NO: 47 - protein, 11 aa, Artificial Sequence
  Synthetic polypeptide
YGRKKRRQRRR

SEQ ID NO: 48 - protein, 12 aa, Artificial Sequence
  Synthetic polypeptide
RRQRRTSKLMKR

SEQ ID NO: 49 - protein, 27 aa, Artificial Sequence
  Synthetic polypeptide
GWTLNSAGYLLGKINLKALAALAKKIL

SEQ ID NO: 50 - protein, 33 aa, Artificial Sequence
  Synthetic polypeptide
KALAWEAKLAKALAKALAKHLAKALAKALKCEA

SEQ ID NO: 51 - protein, 16 aa, Artificial Sequence
  Synthetic polypeptide
RQIKIWFQNRRMKWKK

SEQ ID NO: 52 - protein, 9 aa, Artificial Sequence
  Synthetic polypeptide
RKKRRQRRR

SEQ ID NO: 53 - protein, 8 aa, Artificial Sequence
  Synthetic polypeptide
RKKRRQRR

SEQ ID NO: 54 - protein, 11 aa, Artificial Sequence
  Synthetic polypeptide
YARAAARQARA

SEQ ID NO: 55 - protein, 11 aa, Artificial Sequence
  Synthetic polypeptide
THRLPRRRRRR

SEQ ID NO: 56 - protein, 11 aa, Artificial Sequence
  Synthetic polypeptide
GGRRARRRRRR

SEQ ID NO: 57 - protein, 36 aa, Artificial Sequence
  Synthetic polypeptide
YTSLIHSLIEESQNQQEKNEQELLELDKWASLWNWF

SEQ ID NO: 58 - protein, 10 aa, Artificial Sequence
  Synthetic polypeptide
CYPYDVPDYA

SEQ ID NO: 59 - protein, 8 aa, Artificial Sequence
  Synthetic polypeptide
DYKDDDDK

SEQ ID NO: 60 - protein, 10 aa, Artificial Sequence
  Synthetic polypeptide
EQKLISEEDL

SEQ ID NO: 61 - protein, 10 aa, Artificial Sequence
  Synthetic polypeptide
SYGRKKRRQR

SEQ ID NO: 62 - DNA, 30 bp, Artificial Sequence
  Synthetic oligonucleotide
tcctatggcaggaagaagcggagacagcga

SEQ ID NO: 63 - DNA, 21 bp, Artificial Sequence
  Synthetic oligonucleotide
catgtgcctgtttctgccatg

SEQ ID NO: 64 - DNA, 19 bp, Artificial Sequence
  Synthetic oligonucleotide
gtcaagtctcacttgcgtc

SEQ ID NO: 65 - DNA, 19 bp, Artificial Sequence
  Synthetic oligonucleotide
cttacgctgagtacttcga

SEQ ID NO: 66 - protein, 11 aa, Artificial Sequence
  Synthetic polypeptide
RKAKMRRQRRR

SEQ ID NO: 67 - protein, 10 aa, Artificial Sequence
  Synthetic polypeptide
RKAKRRQRRR

SEQ ID NO: 68 - DNA, 20 bp, Artificial Sequence
  Synthetic oligonucleotide
gagccctcagatcctgcata

SEQ ID NO: 69 - DNA, 20 bp, Artificial Sequence
  Synthetic oligonucleotide
agctcctctggtttcccttt

SEQ ID NO: 70 - protein, 8 aa, Artificial Sequence
  Synthetic polypeptide
GRKKRRQR

SEQ ID NO: 71 - protein, 8 aa, Artificial Sequence
  Synthetic polypeptide
KKRRQRRR

SEQ ID NO: 72 - protein, 112 aa, Artificial Sequence
  Synthetic polypeptide
MEPVDPRLEPWKHPGSQPKTACTNCYCKKCCFHCQVCFITKALGISYGRKKRRQRRRPPQGSQTHQVSLSKQPTSQSRGDPTGPKESKKKVERETETDPFDGGDYKDDDDKD

(19) United States
(12) Patent Application Publication
(10) Pub. No.: US 2012/0108500 A1
(43) Pub. Date: May 3, 2012

(54) Compositions and Methods for Modulating Immunodeficiency Virus Transcription

(76) Inventors: Naoki Sakane, Kanazawa-ku Yokohama (JP); Melanie Ott, Mill Valley, CA (US)

(21) Appl. No.: 13/252,624

(22) Filed: Oct. 4, 2011

Related U.S. Application Data
(60) Provisional application No. 61/431,241, filed on Jan. 10, 2011.
(60) Provisional application No. 61/390,968, filed on Oct. 7, 2010.

(51) Int. Cl. A61K 38/02; A61P 31/18; C12Q 1/48; C12N 5/07; A61K 31/7088

(52) U.S. Cl. 514/3.8; 435/375; 514/44 A; 435/15

(57) ABSTRACT

The present disclosure provides methods of modulating immunodeficiency virus transcription, involving modulating enzymatic activity and/or levels of a lysine-specific demethylase-1 (LSD1) polypeptide and/or LSD1-mediated demethylation of methylated Tat. The present disclosure also provides method of identifying agents that modulate LSD1-mediated demethylation of a human immunodeficiency virus (HIV) Tat polypeptide.

### CROSS-REFERENCE

This application claims the benefit of U.S. Provisional Patent Application No. 61/390,968, filed Oct. 7, 2010, and U.S. Provisional Patent Application No. 61/431,241, filed Jan. 10, 2011, which applications are incorporated herein by reference in their entirety.

### BACKGROUND

Lysine-specific demethylase-1 (LSD1) has been shown to exhibit histone demethylation activity, and has been implicated in processes such as carcinogenesis and inflammation.

Human immunodeficiency virus (HIV) infection is a pressing threat to public health worldwide. According to UNAIDS estimates, as of the end of 2007, 33.2 million persons were infected with HIV-1 worldwide, 2.5 million of those becoming newly infected and another 2.1 million dying of HIV-related deaths in that year alone.

### LITERATURE

U.S. Patent Publication No. 2008/0070257; U.S. Patent Publication No. 2009/0170796; U.S. Pat. No. 7,741,086; Huang et al. (2007) Proc. Natl. Acad. Sci. USA 104:8023; Kahl et al. (2006) Cancer Res. 66:1341; Reddy et al. (2008) Circ. Res. 103:615; Wang et al. (2007) Nature 882; Shi et al. (2005) Mol. Cell. 19:857.

### SUMMARY OF THE INVENTION

The present disclosure provides methods of modulating immunodeficiency virus transcription, involving modulating enzymatic activity and/or levels of a lysine-specific demethylase-1 (LSD1) polypeptide and/or LSD1-mediated demethylation of methylated Tat. The present disclosure also provides method of identifying agents that modulate LSD1-mediated demethylation of a human immunodeficiency virus (HIV) Tat polypeptide.

### BRIEF DESCRIPTION OF THE DRAWINGS

FIGS. 1A-D depict in vitro acetylation and methylation assays using synthetic Tat peptides.

FIGS. 2A-C depict MALDI-TOF mass spectrometric analysis of cellular Tat. FIG. 2A shows the sequence of a Tat-FLAG peptide (SEQ ID NO:72); FIG. 2B shows GRKKRRQR (SEQ ID NO:70); and FIG. 2C shows KKRRQRRR (SEQ ID NO:71).

FIG. 3A-C depict analysis of K50Ac/K51Me Tat using anti-Tat antibodies.

FIGS. 4A-D depict demethylation of monomethylated K51 Tat by LSD1.

FIGS. 5A-C depict in vivo recruitment of LSD1 and CoREST to HIV long terminal repeat (LTR).

FIGS. 6A-F depict activation of Tat-dependent HIV transcription by LSD1/CoREST complex.

FIGS. 7A and 7B depict inhibition of HIV transcription by phenelzine in cultured cells (FIG. 7A) and in primary CD4⁺ T cells.

FIG. 8 provides an amino acid sequence of human LSD1 isoform a.

FIGS. 9A and 9B provide a nucleotide sequence encoding human LSD1 isoform a.

FIG. 10 provides an amino acid sequence of human LSD1 isoform b.

FIGS. 11A and 11B provide a nucleotide sequence encoding human LSD1 isoform b.

FIG. 12 provides a consensus Tat amino acid sequence.

FIGS. 13A and 13B provide Tat amino acid sequences.

FIG. 14 provides a nucleotide sequence encoding a Tat polypeptide.

FIG. 15 provides an amino acid sequence of a CoREST polypeptide (SEQ ID NO:8).

FIG. 16 provides a nucleotide sequence encoding a CoREST polypeptide.

FIG. 17 provides an amino acid sequence of a BHC80 polypeptide.

FIGS. 18A and 18B provide a nucleotide sequence encoding a BHC80 polypeptide.

### DEFINITIONS

The term “immunodeficiency virus” includes human immunodeficiency virus (HIV), feline immunodeficiency virus, and simian immunodeficiency virus. The term “human immunodeficiency virus” as used herein, refers to human immunodeficiency virus-1 (HIV-1); human immunodeficiency virus-2 (HIV-2); and any of a variety of HIV subtypes and quasispecies.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of a compound that, when administered to a mammal or other subject for treating a disease, is sufficient to effect such treatment for the disease. The “therapeutically effective amount” will vary depending on the compound or the cell, the disease and its severity and the age, weight, etc., of the subject to be treated.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The terms “polypeptide,” “peptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. In keeping with standard polypeptide nomenclature, J. Biol. Chem., 243 (1969), 3552-59 is used.

The terms “polynucleotide” and “nucleic acid” are used interchangeably herein to refer to polymeric forms of nucleotides of any length. The polynucleotides may contain deoxyribonucleotides, ribonucleotides, and/or their analogs.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and is free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal and the like. In some embodiments the composition is suitable for administration by a transdermal route, using a penetration enhancer other than dimethylsulfoxide (DMSO). In other embodiments, the pharmaceutical compositions are suitable for administration by a route other than transdermal administration. A pharmaceutical composition will in some embodiments include a subject compound and a pharmaceutically acceptable excipient. In some embodiments, a pharmaceutically acceptable excipient is other than DMSO.

As used herein, “pharmaceutically acceptable derivatives” of a compound of the invention include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and are either pharmaceutically active or are prodrugs.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

A “pharmaceutically acceptable ester” of a compound of the invention means an ester that is pharmaceutically acceptable and that possesses the desired pharmacological activity of the parent compound, and includes, but is not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

A “pharmaceutically acceptable enol ether” of a compound of the invention means an enol ether that is pharmaceutically acceptable and that possesses the desired pharmacological activity of the parent compound, and includes, but is not limited to, derivatives of formula C═C(OR) where R is hydrogen, alkyl, alkenyl, alkynyl, aryl, heteroaryl, aralkyl, heteroaralkyl, cycloalkyl or heterocyclyl.

A “pharmaceutically acceptable solvate or hydrate” of a compound of the invention means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

“Pro-drugs” means any compound that releases an active parent drug according to one or more of the generic formulas shown below in vivo when such prodrug is administered to a mammalian subject. Prodrugs of a compound of one or more of the generic formulas shown below are prepared by modifying functional groups present in the compound of the generic formula in such a way that the modifications may be cleaved in vivo to release the parent compound. Prodrugs include compounds of one or more of the generic formulas shown below wherein a hydroxy, amino, or sulfhydryl group in one or more of the generic formulas shown below is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylaminocarbonyl) of hydroxy functional groups in compounds of one or more of the generic formulas shown below, and the like.

The term “organic group” and “organic radical” as used herein means any carbon-containing group, including hydrocarbon groups that are classified as an aliphatic group, cyclic group, aromatic group, functionalized derivatives thereof and/or various combinations thereof. The term “aliphatic group” means a saturated or unsaturated linear or branched hydrocarbon group and encompasses alkyl, alkenyl, and alkynyl groups, for example. The term “alkyl group” means a substituted or unsubstituted, saturated linear or branched hydrocarbon group or chain (e.g., C₁ to C₈) including, for example, methyl, ethyl, isopropyl, tert-butyl, heptyl, iso-propyl, n-octyl, dodecyl, octadecyl, amyl, 2-ethylhexyl, and the like. Suitable substituents include carboxy, protected carboxy, amino, protected amino, halo, hydroxy, protected hydroxy, nitro, cyano, monosubstituted amino, protected monosubstituted amino, disubstituted amino, C₁ to C₇ alkoxy, C₁ to C₇ acyl, C₁ to C₇ acyloxy, and the like. The term “substituted alkyl” means the above defined alkyl group substituted from one to three times by a hydroxy, protected hydroxy, amino, protected amino, cyano, halo, trifloromethyl, mono-substituted amino, di-substituted amino, lower alkoxy, lower alkylthio, carboxy, protected carboxy, or a carboxy, amino, and/or hydroxy salt. As used in conjunction with the substituents for the heteroaryl rings, the terms “substituted (cycloalkyl)alkyl” and “substituted cycloalkyl” are as defined below substituted with the same groups as listed for a “substituted alkyl” group. The term “alkenyl group” means an unsaturated, linear or branched hydrocarbon group with one or more carbon-carbon double bonds, such as a vinyl group. The term “alkynyl group” means an unsaturated, linear or branched hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polycyclic aromatic hydrocarbon group, and may include one or more heteroatoms, and which are further defined below. The term “heterocyclic group” means a closed ring hydrocarbon in which one or more of the atoms in the ring are an element other than carbon (e.g., nitrogen, oxygen, sulfur, etc.), and are further defined below.

“Organic groups” may be functionalized or otherwise comprise additional functionalities associated with the organic group, such as carboxyl, amino, hydroxyl, and the like, which may be protected or unprotected. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ethers, esters, haloalkyls, nitroalkyls, carboxyalkyls, hydroxyalkyls, sulfoalkyls, etc.

In addition to the disclosure herein, any of the organic groups used herein may be substituted or unsubstituted. The term “substituted,” when used to modify a specified group or radical, can also mean that one or more hydrogen atoms of the specified group or radical are each, independently of one another, replaced with the same or different substituent groups as defined herein.

The terms “halo” and “halogen” refer to the fluoro, chloro, bromo or iodo groups. There can be one or more halogen, which are the same or different. Halogens of particular interest include chloro and bromo groups.

The term “haloalkyl” refers to an alkyl group as defined above that is substituted by one or more halogen atoms. The halogen atoms may be the same or different. The term “dihaloalkyl” refers to an alkyl group as described above that is substituted by two halo groups, which may be the same or different. The term “dihaloalkyl” refers to an alkyl group as describe above that is substituted by three halo groups, which may be the same or different. The term “perhaloalkyl” refers to a haloalkyl group as defined above wherein each hydrogen atom in the alkyl group has been replaced by a halogen atom. The term “perfluoroalkyl” refers to a haloalkyl group as defined above wherein each hydrogen atom in the alkyl group has been replaced by a fluoro group.

The term “cycloalkyl” means a mono-, bi-, or tricyclic saturated ring that is fully saturated or partially unsaturated. Examples of such a group included cyclopropyl, cyclobutyl, cyclopentyl, cyclohexyl, cycloheptyl, adamantyl, cyclooctyl, cis- or trans decalin, bicyclo[2.2.1]hept-2-ene, cyclohex-1-enyl, cyclopent-1-enyl, 1,4-cyclooctadienyl, and the like.

The term “(cycloalkyl)alkyl” means the above-defined alkyl group substituted for one of the above cycloalkyl rings. Examples of such a group include (cyclohexyl)methyl, 3-(cyclopropyl)-n-propyl, 5-(cyclopentyl)hexyl, 6-(adamantyl)hexyl, and the like.

The term “substituted phenyl” specifies a phenyl group substituted with one or more moieties, and in some instances one, two, or three moieties, chosen from the groups consisting of halogen, hydroxy, protected hydroxy, cyano, nitro, trifluoromethyl, C₁ to C₇ alkyl, C₁ to C₇ alkoxy, C₁ to C₇ acyl, C₁ to C₇ acyloxy, carboxy, oxycarboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino or phenyl, substituted or unsubstituted, such that, for example, a biphenyl or naphthyl group results.

Examples of the term “substituted phenyl” includes a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3, or 4-hydroxyphenyl, 2,4-dihydroxyphenyl, the protected-hydroxy derivatives thereof and the like; a nitrophenyl group such as 2, 3, or 4-nitrophenyl; a cyanophenyl group, for example, 2, 3 or 4-cyanophenyl; a mono- or di(alkyl)phenyl group such as 2, 3, or 4-methylphenyl, 2,4-dimethylphenyl, 2, 3 or 4-(iso-propyl)phenyl, 2, 3, or 4-ethylphenyl, 2, 3 or 4-(n-propyl)phenyl and the like; a mono or di(alkoxy)phenyl group, for example, 2,6-dimethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono- or di(hydroxymethyl)phenyl or (protected hydroxymethyl)phenyl such as 2, 3 or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-4-ethyl-2-hydroxyphenyl, 3-hydroxy-4-nitrophenyl, 2-hydroxy-4-chlorophenyl and the like.

The term “(substituted phenyl)alkyl” means one of the above substituted phenyl groups attached to one of the above-described alkyl groups. Examples of include such groups as 2-phenyl-1-chloroethyl, 2-(4′-methoxyphenyl)ethyl, 4-(2′,6′-dihydroxy phenyl)ₙ-hexyl, 2-(5′-cyano-3′-methoxyphenyl)n-pentyl, 3-(2′,6′-dimethylphenyl)n-propyl, 4-chloro-3-aminobenzyl, 6-(4′-methoxyphenyl)-3-carboxy(n-hexyl), 5-(4′-aminomethylphenyl)-3-(aminomethyl)n-pentyl, 5-phenyl-3-oxo-n-pent-1-yl, (4-hydroxynapth-2-yl)methyl and the like.

As noted above, the term “aromatic” or “aryl” refers to six membered carbocyclic rings. Also as noted above, the term “heteroaryl” denotes optionally substituted five-membered or six-membered rings that have 1 to 4 heteroatoms, such as oxygen, sulfur and/or nitrogen atoms, in particular nitrogen, either alone or in conjunction with sulfur or oxygen ring atoms.

Furthermore, the above optionally substituted five-membered or six-membered rings can optionally be fused to an aromatic 5-membered or 6-membered ring system. For example, the rings can be optionally fused to an aromatic 5-membered or 6-membered ring system such as a pyridine or a triazole system, and preferably to a benzene ring.

The following ring systems are examples of the heterocyclic (whether substituted or unsubstituted) radicals denoted by the term “heteroaryl”: thienyl, furyl, pyrrolyl, pyrrolidinyl, imidazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, oxazinyl, triazinyl, thiadiazinyl tetrazolo, 1,5-[b]pyridazinyl and purinyl, as well as benzo-fused derivatives, for example, benzoxazolyl, benzthiazolyl, benzimidazolyl and indolyl.

Substituents for the above optionally substituted heteroaryl rings are from one to three halo, trihalomethyl, amino, protected amino, amino salts, mono-substituted amino, di-substituted amino, carboxy, protected carboxy, carboxylate salts, hydroxy, protected hydroxy, salts of a hydroxy group, lower alkoxy, lower alkylthio, alkyl, substituted alkyl, cycloalkyl, substituted cycloalkyl, (cycloalkyl)alkyl, substituted (cycloalkyl)alkyl, phenyl, substituted phenyl, phenylalkyl, and (substituted phenyl)alkyl. Substituents for the heteroaryl group are as heretofore defined, or in the case of trihalomethyl, can be trifluoromethyl, trichloromethyl, tribromomethyl, or triiodomethyl. As used in conjunction with the above substituents for heteroaryl rings, “lower alkoxy” means a C₁ to C₄ alkoxy group, similarly, “lower alkylthio” means a C₁ to C₄ alkylthio group.

The term “(monosubstituted)amino” refers to an amino group with one substituent chosen from the group consisting of phenyl, substituted phenyl, alkyl, substituted alkyl, C₁ to C₄ acyl, C₂ to C₇ alkenyl, C₂ to C₇ substituted alkenyl, C₂ to C₇ alkynyl, C₇ to C₁₆ alkylaryl, C₇ to C₁₆ substituted alkylaryl and heteroaryl group. The (monosubstituted) amino can additionally have an amino-protecting group as encompassed by the term “protected (monosubstituted)amino.” The term “(disubstituted)amino” refers to amino groups with two substituents chosen from the group consisting of phenyl, substituted phenyl, alkyl, substituted alkyl, C₁ to C₇ acyl, C₂ to C₇ alkenyl, C₂ to C₇ alkynyl, C₇ to C₁₆ alkylaryl, C₇ to C₁₆ substituted alkylaryl and heteroaryl. The two substituents can be the same or different.

The term “heteroaryl(alkyl)” denotes an alkyl group as defined above, substituted at any position by a heteroaryl group, as above defined.

“Optional” or “optionally” means that the subsequently described event, circumstance, feature, or element may, but need not, occur, and that the description includes instances where the event or circumstance occurs and instances in which it does not. For example, “heterocyclo group optionally mono- or di-substituted with an alkyl group” means that the alkyl may, but need not, be present, and the description includes situations where the heterocyclo group is mono- or disubstituted with an alkyl group and situations where the heterocyclo group is not substituted with the alkyl group.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

A compound may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see, e.g., the discussion in Chapter 4 of “Advanced Organic Chemistry”, 4th edition J. March, John Wiley and Sons, New York, 1992).

“In combination with” or “co-administered” or “co-administration” as used herein refers to uses where, for example, a first compound is administered during the entire course of administration of a second compound; where the first compound is administered for a period of time that is overlapping with the administration of the second compound, e.g. where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits; ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an immunodeficiency virus” includes a plurality of such immunodeficiency viruses and reference to “the active agent” includes reference to one or more active agents and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of modulating immunodeficiency virus transcription, involving modulating enzymatic activity and/or levels of a lysine-specific demethylase-1 (LSD1) polypeptide and/or modulating LSD1-mediated demethylation of a Tat polypeptide. The present disclosure also provides method of identifying agents that modulate LSD1-mediated demethylation of a human immunodeficiency virus (HIV) Tat polypeptide.

It has been observed that LSD1 is an activator of HIV transcription and latency, and that LSD1 inhibitors suppress HIV transcription. The present disclosure provides methods of inhibiting immunodeficiency virus transcription in a cell infected with the immunodeficiency virus, the methods generally involving contacting the cell with an agent that inhibits enzymatic activity of LSD1 and/or that reduces the level of LSD1 in the cell and/or that inhibits LSD1-mediated demethylation of a Tat polypeptide in the cell. The methods are useful for treating an immunodeficiency virus infection in an individual. Thus, the present disclosure further provides methods of treating an immunodeficiency virus infection in an individual, the methods generally involving administering to the individual an effective amount of an agent that inhibits enzymatic activity of LSD1 and/or that reduces the level of LSD1 in an immunodeficiency virus-infected cell in the individual and/or that inhibits LSD1-mediated demethylation of a Tat polypeptide in an immunodeficiency virus-infected cell in the individual.

The present disclosure provides methods of reactivating latent HIV integrated into the genome of an HIV-infected cell. The methods generally involve contacting an HIV-infected cell in which HIV is latent with an agent that increases LSD1 enzymatic activity in the cell. Latently infected cells contain replication-competent integrated HIV-1 genomes that are blocked at the transcriptional level, resulting in the absence of viral protein expression. The present disclosure provides methods for reducing the reservoir of latent immunodeficiency virus in an individual.

### Methods of Modulating Immunodeficiency Virus Transcription

The present disclosure provides methods of modulating immunodeficiency virus transcription in a cell infected with the immunodeficiency virus, the methods generally involving contacting the cell with an agent that modulates enzymatic activity of LSD1 and/or that reduces the level of LSD1 in the cell and/or that inhibits LSD1-mediated demethylation of a Tat polypeptide in the cell.

Agents that reduce LSD1 enzymatic activity include: 1) small molecule agents that inhibit LSD1 enzymatic activity; and 2) BHC80 polypeptides. Agents that reduce the level of LSD1 in a cell include: 1) inhibitory nucleic acid agents that specifically reduce LSD1 expression; 2) dominant negative CoREST polypeptides; and 3) inhibitory nucleic acid agents that reduce CoREST expression. Agents that inhibit LSD1-mediated demethylation of methylated Tat include HDAC inhibitors. In some cases, a suitable agent is an agent that targets CoREST independently of any direct effect on LSD1.

Methods of inhibiting immunodeficiency virus transcription are provided, where a subject method of inhibiting immunodeficiency virus transcription involves contacting a cell infected with an immunodeficiency virus with an agent that inhibits enzymatic activity of LSD1 in the cell and/or that reduces the level of LSD1 in the cell and/or that inhibits LSD1-mediated demethylation of a Tat polypeptide in the cell. Such methods are useful for treating an immunodeficiency virus infection in an individual.

Methods of activating latent immunodeficiency virus present in the genome of a cell infected with the immunodeficiency virus are also provided, where a subject method of activating latent immunodeficiency virus involves contacting a cell having an immunodeficiency virus latent in the genome of the cell with an agent that increases activity of LSD1 in the cell and/or that increases the level of LSD1 in the cell.

### Methods of Inhibiting Immunodeficiency Virus Transcription

The present disclosure provides methods of inhibiting immunodeficiency virus transcription in a cell infected with the immunodeficiency virus, the methods generally involving contacting the cell with an agent that inhibits enzymatic activity of LSD1 and/or that reduces the level of LSD1 in the cell and/or that inhibits LSD1-mediated demethylation of a Tat polypeptide in the cell and/or that inhibits activity of a CoREST component independently of any direct effect on LSD1.

A suitable agent that decreases enzymatic activity of an LSD1 polypeptide decreases enzymatic activity of an LSD1 polypeptide by at least about 10%, at least about 20%, at least about 25%, at least about 30%, at least about 40%, at least about 50%, at least about 60%, at least about 70%, at least about 80%, or greater than 80%, compared to the level of enzymatic activity of the LSD1 polypeptide in the absence of the agent.

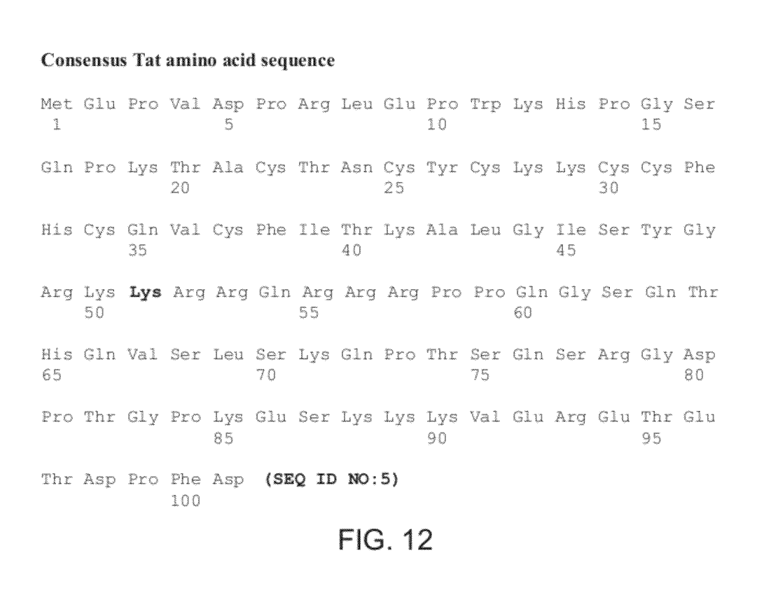

Enzymatic activity of an LSD1 polypeptide includes demethylation of Lys-51 of a Tat polypeptide, e.g., demethylation of Tat polypeptide lysine residue corresponding to Lys-51 of the consensus Tat amino acid sequence depicted in FIG. 12 and set forth in SEQ ID NO:5. A Tat lysine residue at a position corresponding to Lys-51 of the consensus Tat sequence can be monomethylated or dimethylated. Demethylation includes removal of the methyl group of monomethylated Tat; and removal of one or both methyl groups of dimethylated Tat.

A suitable agent that that reduces the level of LSD1 in the cell reduces the level of LSD1 in the cell by at least about 10%, at least about 20%, at least about 25%, at least about 30%, at least about 40%, at least about 50%, at least about 60%, at least about 70%, at least about 80%, or greater than 80%, compared to the level of LSD1 in the cell in the absence of the agent.

A suitable agent that reduces LSD1-mediated demethylation of a methylated Tat polypeptide in a cell reduces LSD1-mediated demethylation of the methylated Tat polypeptide by at least about 10%, at least about 20%, at least about 25%, at least about 30%, at least about 40%, at least about 50%, at least about 60%, at least about 70%, at least about 80%, or greater than 80%, compared to the LSD1-mediated demethylation of the methylated Tat polypeptide in a cell in the absence of the agent. For example, a suitable agent that reduces LSD1-mediated demethylation of a methylated Tat polypeptide in a cell reduces demethylation of Tat polypeptide lysine residue corresponding to Lys-51 of the consensus Tat amino acid sequence depicted in FIG. 12 and set forth in SEQ ID NO:5. Methods for determining the level of methylated Tat polypeptide in a cell are known in the art, and are described in detail below, including in the Examples section. For example, the amount of methylated Tat polypeptide in a cell can be determined using antibody specific for methylated Tat. A cell that produces methylated Tat polypeptide can be contacted with an agent; and the effect of the agent on the level of LSD1-mediated demethylation of Tat can be determined using antibody specific for methylated Tat. A methylated arginine-rich motif (ARM) Tat peptide can be used, where a suitable methylated ARM peptide has the sequence RKK^{Me}RRQRRR (SEQ ID NO:35), where K^{Me} is monomethylated lysine.

An agent that decreases enzymatic activity of an LSD1 polypeptide in a cell and/or that reduces the level of LSD1 in the cell decreases transcription of an immunodeficiency virus in the cell by at least about 10%, at least about 20%, at least about 25%, at least about 30%, at least about 40%, at least about 50%, at least about 60%, at least about 70%, at least about 80%, or greater than 80%, compared to the level of transcription of the immunodeficiency virus in the cell in the absence of the agent. Whether immunodeficiency virus transcription in a cell is reduced can be determined by a number of well-known methods including, e.g., established methods for detection of immunodeficiency virus RNA and/or a cDNA copy of immunodeficiency virus RNA. Such methods include, e.g., a polymerase chain reaction (PCR) method, a reverse transcription-PCR (RT-PCR) method, and the like.

### LSD1 Polypeptides

An “LSD1 polypeptide” is a polypeptide having substantial amino acid sequence identity to a known LSD1 polypeptide; and having enzymatic activity, including, e.g., the ability to remove a methyl group from a substrate polypeptide such as a histone (e.g., demethylates Lys-4 from histone H3), an HIV Tat polypeptide (e.g., demethylates Lys-51 of HIV Tat), and the like. Amino acid sequences of LSD1 polypeptides, and nucleotide sequences encoding such polypeptides, from a number of species are publicly available. See, e.g., 1) GenBank Accession No. XP_{—}866610.1 for a Canis familiaris LSD1 amino acid sequence; and GenBank Accession No. XM_{—}861517.1 for a nucleotide sequence encoding the amino acid sequence provided at GenBank Accession No. XP_{—}866610.1; 2) GenBank Accession No. XP_{—}513190.2 for a Pan troglodytes LSD1 amino acid sequence; and GenBank Accession No. XM_{—}513190.2 for a nucleotide sequence encoding the amino acid sequence provided at GenBank Accession No. XP_{—}513190.2; 3) GenBank Accession No. XP_{—}575936.2 for a Rattus norvegicus LSD1 amino acid sequence; and GenBank Accession No. XM_{—}575936.2 for a nucleotide sequence encoding the amino acid sequence provided at GenBank Accession No. XP_{—}575936.2; 4) GenBank Accession No. NP_{—}598633.1 for a Mus musculus amino acid sequence; and GenBank Accession No. NM_{—}133872.1 for a nucleotide sequence encoding the amino acid sequence provided at GenBank Accession No. NP_{—}598633.1; 5) GenBank Accession Nos. NP_{—}055828 and NP_{—}00100999 for Homo sapiens LSD1 amino acid sequences; and GenBank Accession Nos. NM_{—}015013 and NM_{—}001009999 for nucleotide sequences encoding the amino acid sequences provided at GenBank Accession Nos. NP_{—}055828 and NP_{—}00100999. A crystal structure of human LSD1 is reported in Chen et al. (2006) Proc. Natl. Acad. Sci. USA 103:13956. See also, Formeris et al. (2005) FEBS Lett. 579:2203.

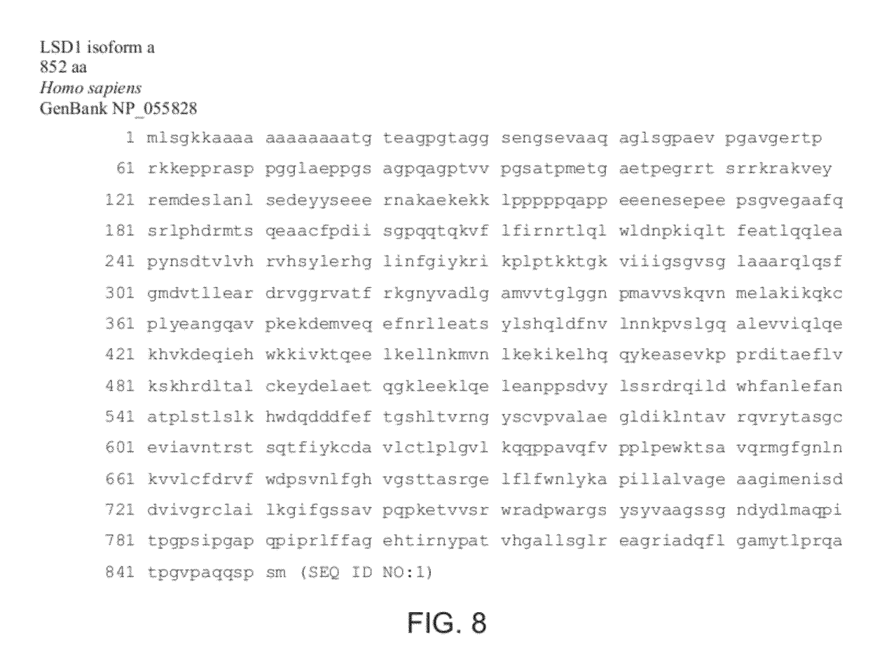

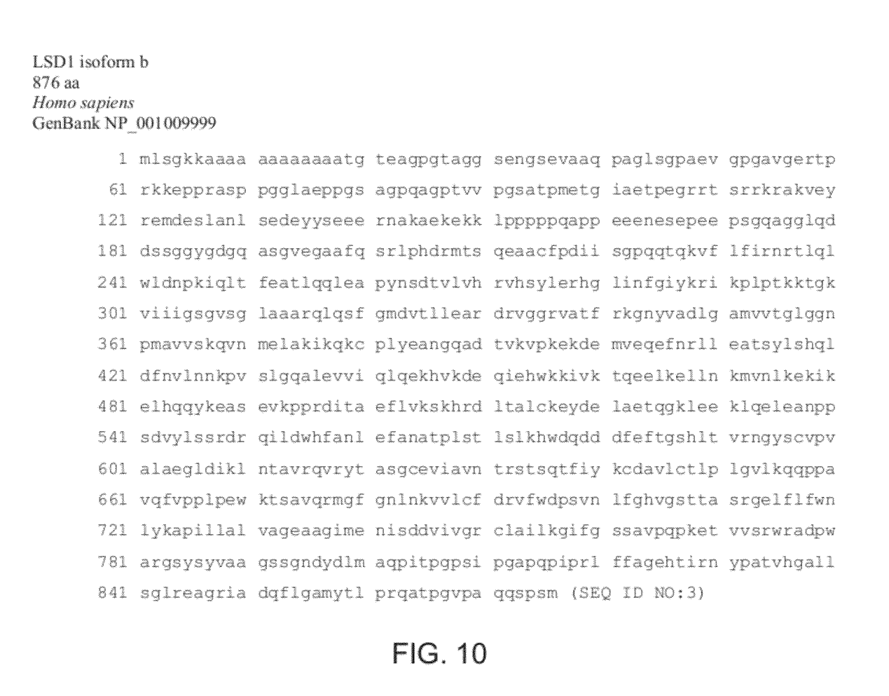

In some embodiments, an LSD1 polypeptide comprises an amino acid sequence having at least about 75%, at least about 80%, at least about 85%, at least about 90%, at least about 95%, at least about 98%, at least about 99%, or 100%, amino acid sequence identity to a contiguous stretch of from about 750 amino acids to about 800 amino acids, or from about 800 amino acids to 852 amino acids, of the amino acid sequence depicted in FIG. 8 and set forth in SEQ ID NO:1. In some embodiments, an LSD1 polypeptide comprises an amino acid sequence having at least about 75%, at least about 80%, at least about 85%, at least about 90%, at least about 95%, at least about 98%, at least about 99%, or 100%, amino acid sequence identity to a contiguous stretch of from about 750 amino acids to about 800 amino acids, from about 800 amino acids to about 850 amino acids, or from about 850 amino acids to 876 amino acids, of the amino acid sequence depicted in FIG. 10 and set forth in SEQ ID NO:3.

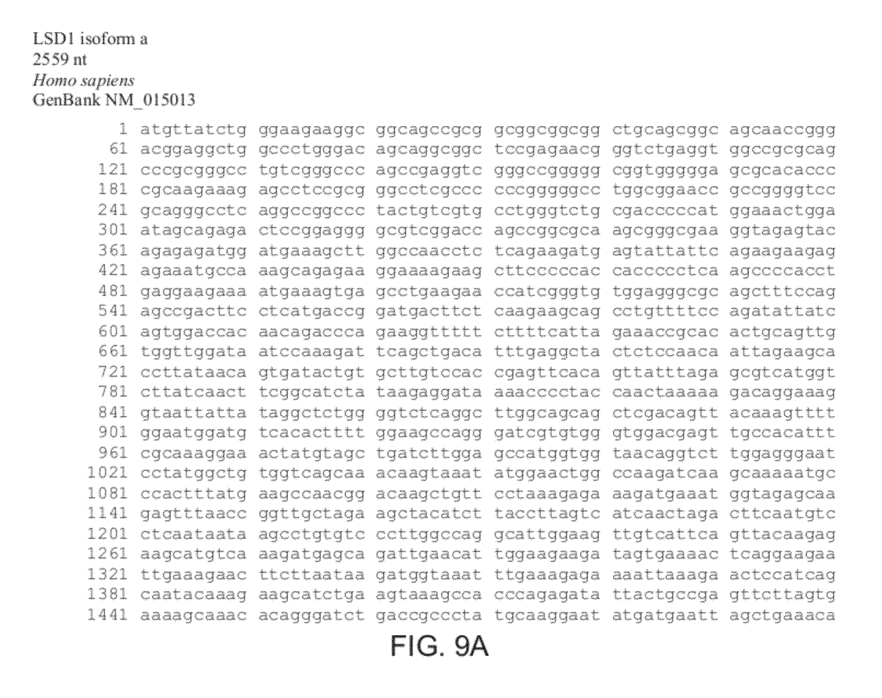

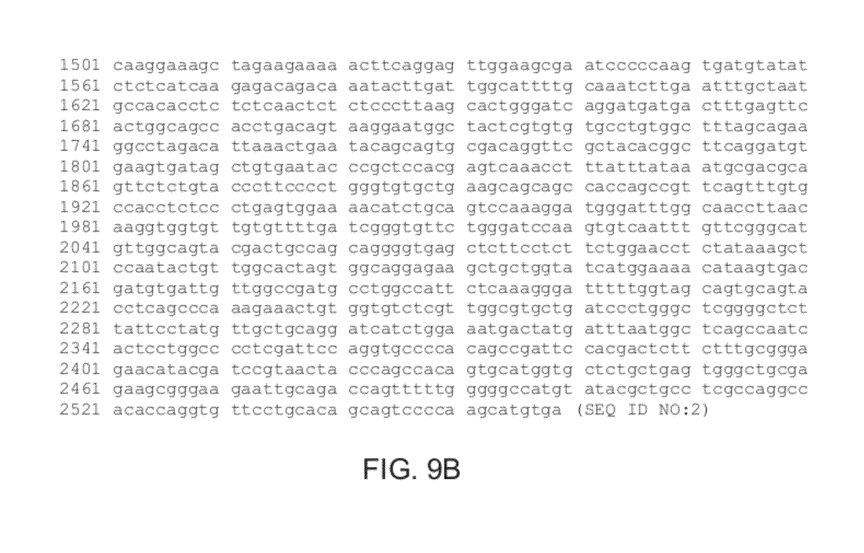

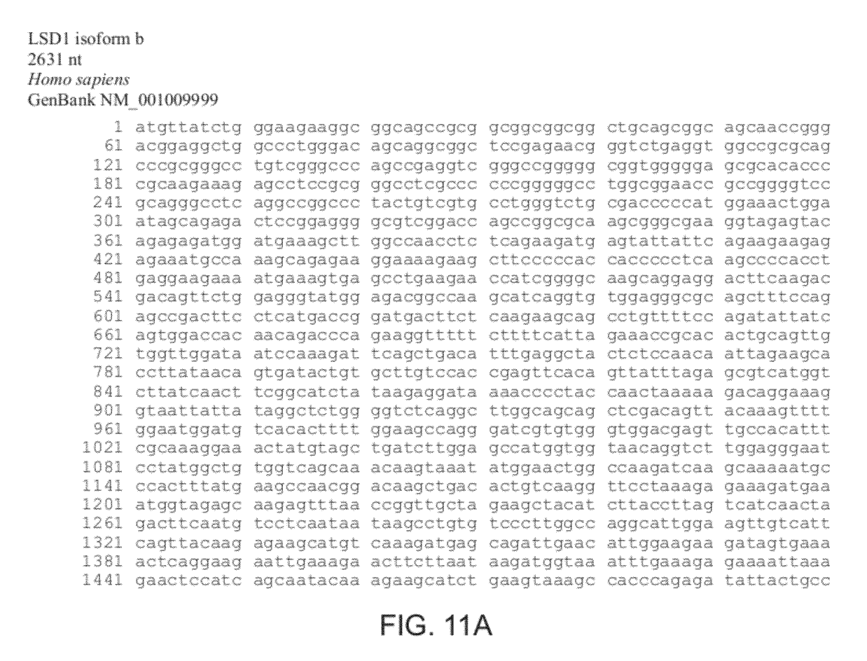

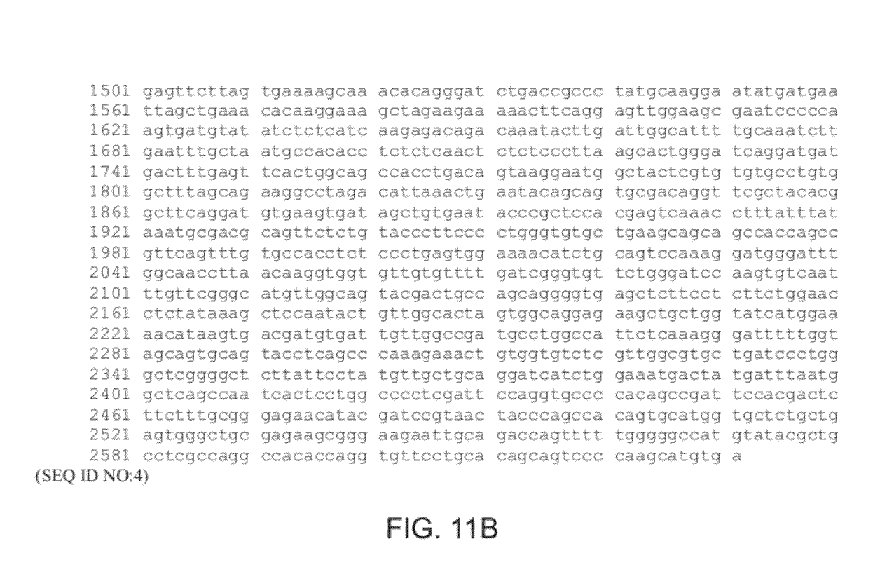

In some embodiments, an LSD1 polypeptide is encoded by a nucleotide sequence having at least about 75%, at least about 80%, at least about 85%, at least about 90%, at least about 95%, at least about 98%, at least about 99%, or 100%, nucleotide sequence identity to a contiguous stretch of from about 2400 nucleotides to about 2500 nucleotides, or from 2500 nucleotides to 2559 nucleotides, of the nucleotide sequence depicted in FIGS. 9A and 9B and set forth in SEQ ID NO:2. In some embodiments, an LSD1 polypeptide is encoded by a nucleotide sequence having at least about 75%, at least about 80%, at least about 85%, at least about 90%, at least about 95%, at least about 98%, at least about 99%, or 100%, nucleotide sequence identity to a contiguous stretch of from about 2500 nucleotides to about 2600 nucleotides, or from 2600 nucleotides to 2631 nucleotides, of the nucleotide sequence depicted in FIGS. 11A and 11B and set forth in SEQ ID NO:4.

### Tat Polypeptides

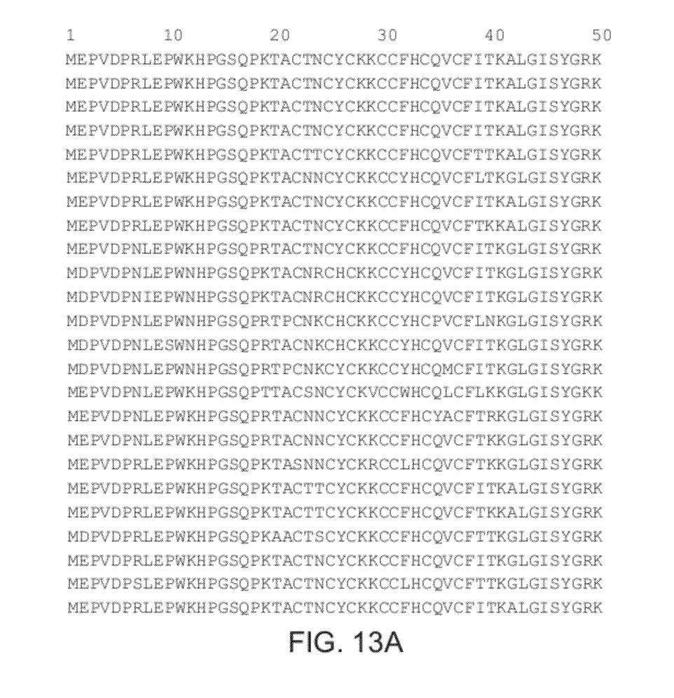

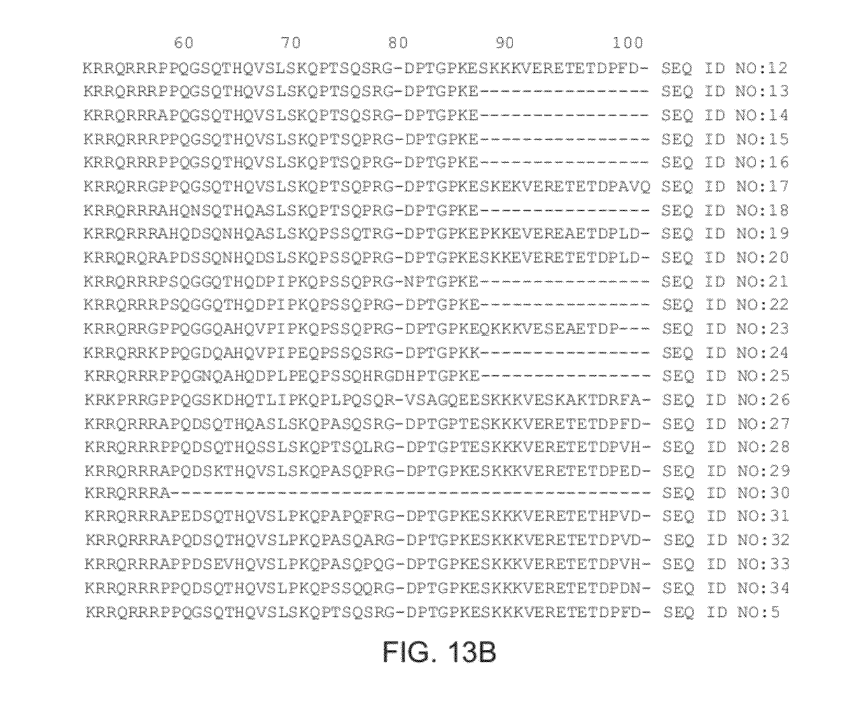

The amino acid sequences of HIV Tat polypeptides are known, and any of these sequences can be included in a subject acetylated Tat polypeptide. Numerous HIV Tat protein amino acid sequences are found under GenBank. Exemplary, non-limiting, HIV Tat protein amino acid sequences are found under GenBank Accession Nos. AAO26250, AAO26252, AAO26254, AAO26258, AAO26260, AAO26262, AAO26264, AAO26266, AAO26268, AAO26270, AAO26272, AAO26274, AAO26276, AAO26278, AAO26280, AAO26282, AAO26284, AAO26286, AAO26288, AAO26290, AAO26292, AAO26294, AAO26296, AAO26298, AAO26300, AAO26302, AAO26304, AAO26306, AAO26308; AAB50256; AAL12204; AAL12195; AAL12186; AAL12177; AAN47131; AAN47122; AAN47113; AAN47104; AAN03332; AAN3323; AAN03314; AAN03305; AAN03296; AAN03287; AAN03278; AAN31592; AAN64126; AAN64117; AAN64108; AAN64099; AAN64090; AAN64080; K02013; AAL29460; and as shown in FIGS. 13A and 13B (SEQ ID NOs:12-34; and consensus Tat sequence SEQ ID NO:5). Additional HIV Tat amino acid sequences are found in Peloponese et al. (1999) J. Biol. Chem. 274:11473-11478; and Goldstein (1996) Nat. Med. 2:960-964.

A methylated substrate for an LSD1 polypeptide can comprise a monomethylated and/or a dimethylated lysine. In some embodiments, a methylated LSD1 substrate is a methylated HIV Tat polypeptide. In some embodiments, a methylated Tat polypeptide comprises a methylated lysine at a position corresponding to Lys-51 of the amino acid sequence depicted in FIG. 12 and set forth in SEQ ID NO:5, where the methylated lysine is monomethylated or dimethylated. LSD1 demethylates mono- and di methylated lysines, but not trimethylated lysines. In some embodiments, a methylated Tat polypeptide comprises the amino acid sequence SYGRKK^{Me}RRQR (SEQ ID NO:6); where the designation “K^{Me}” is methylated lysine), or a variation thereof, where the methylated lysine is monomethylated or dimethylated.

### CoREST Polypeptides and Nucleic Acids

It has been shown that CoREST activates LSD1, and that CoREST interacts directly with LSD1. As such, reduction of CoREST levels and/or inhibition of CoREST-LSD1 binding results in a reduction of LSD1 levels and/or LSD1 enzymatic activity. In some cases, an agent that binds to a CoREST polypeptide can reduce an activity of the CoREST polypeptide independently of any direct effect on LSD1.

A full-length CoREST polypeptide binds an LSD1 polypeptide, and increases enzymatic activity of the bound LSD1 polypeptide; certain fragments of CoREST bind an LSD1 polypeptide, but do not enhance enzymatic activity of the LSD1 polypeptide. See, e.g., Shi et al. (2005) Mol. Cell. 19:857.

Amino acid sequences of CoREST polypeptides, and nucleotide sequences encoding such polypeptides, from a number of species are publicly available. See, e.g., 1) GenBank Accession No. AAF01498 for a Homo sapiens CoREST amino acid sequence; and GenBank Accession No. AF155595 for a nucleotide sequence encoding the amino acid sequence provided at GenBank Accession No. AAF01498; 2) GenBank Accession No. XP_{—}002825179.1 for a Pongo abelii CoREST amino acid sequence; and GenBank Accession No. XM_{—}002825133 for a nucleotide sequence encoding the amino acid sequence provided at GenBank Accession No. XP_{—}002825179.1; 3) GenBank Accession No. CAB93943 for a Mus musculus CoREST amino acid sequence; and GenBank Accession No. X83587 for a nucleotide sequence encoding the amino acid sequence provided at GenBank Accession No. CAB93943; 4) GenBank Accession No. XP_{—}426467.2 for a Gallus gallus CoREST amino acid sequence; ad GenBank Accession No. XM_{—}42467 for a nucleotide sequence encoding the amino acid sequence provided at GenBank Accession No. XP_{—}426467.2; and 5) GenBank Accession No. EDL97484.1 for a Rattus norvegicus CoREST amino acid sequence. Yang et al. (2006) Mol. Cell. 23:377 presents a crystal structure of a CoREST polypeptide in complex with an LSD1 polypeptide.

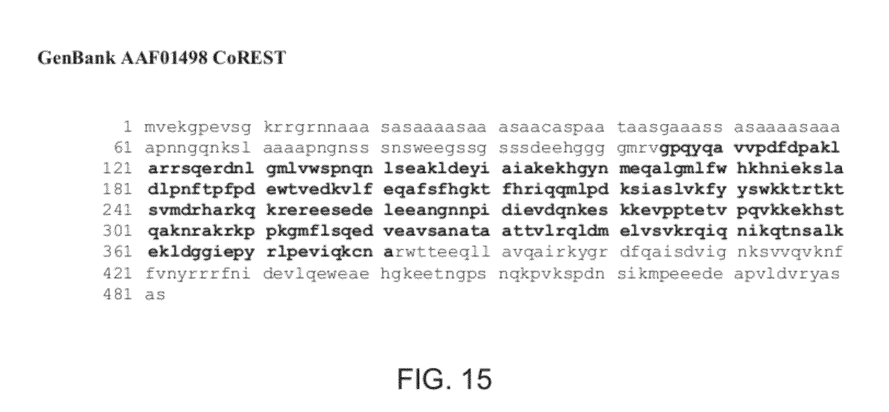

In some embodiments, a CoREST polypeptide comprises an amino acid sequence having at least about 75%, at least about 80%, at least about 85%, at least about 90%, at least about 95%, at least about 98%, at least about 99%, or 100%, amino acid sequence identity to a contiguous stretch of from about 50 to about 75, from about 75 to about 100, from about 100 to about 125, from about 125 to about 150, from about 150 to about 200, from about 200 to about 250, from about 250 to about 275, from about 275 to about 300, from about 300 to about 350, from about 350 to about 400, from about 400 to about 450, or from about 450 to 482, contiguous amino acids of the amino acid sequence depicted in FIG. 15 (SEQ ID NO:8).

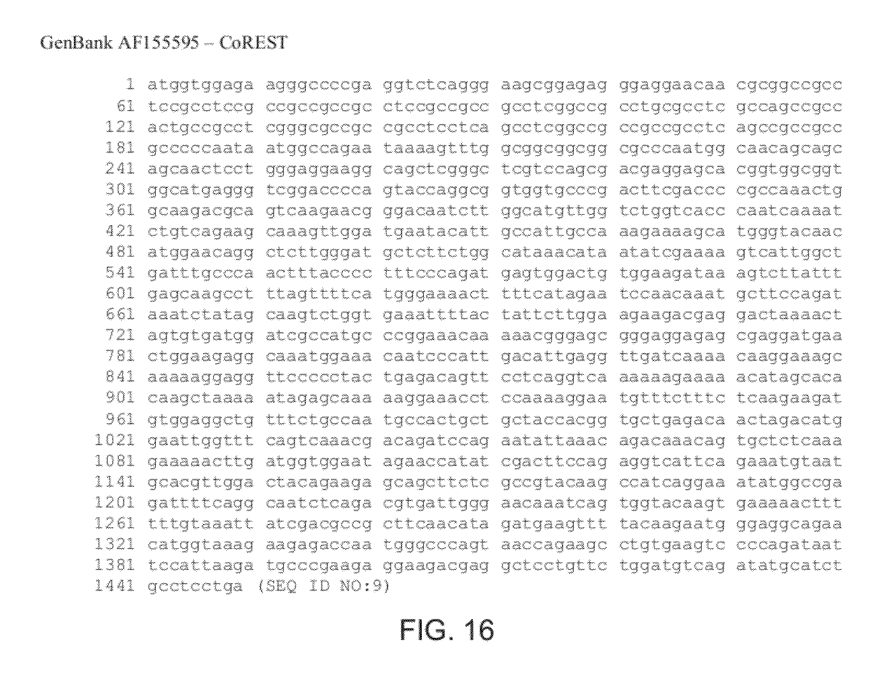

In some embodiments, a CoREST polypeptide is encoded by a nucleotide sequence having at least about 75%, at least about 80%, at least about 85%, at least about 90%, at least about 95%, at least about 98%, at least about 99%, or 100%, nucleotide sequence identity to a contiguous stretch of from about 150 to about 450, from about 450 to about 1000, or from about 1000 to 1449, contiguous nucleotides of the nucleotide sequence depicted in FIG. 16 (SEQ ID NO:9).

As noted above, CoREST nucleic acids (nucleic acids comprising nucleotide sequences encoding CoREST polypeptides) are known in the art. For example, a CoREST nucleic acid comprises a nucleotide sequence having at least about 75%, at least about 80%, at least about 85%, at least about 90%, at least about 95%, at least about 98%, at least about 99%, or 100%, nucleotide sequence identity to a contiguous stretch of from about 150 to about 450, from about 450 to about 1000, or from about 1000 to 1449, contiguous nucleotides of the nucleotide sequence depicted in FIG. 16 (SEQ ID NO:9).

### BHC80 Polypeptides and Nucleic Acids

A BHC80 polypeptide forms part of a complex with CoREST and LSD1. BHC80 is also known as PHD finger protein 21A. A BHC80 polypeptide can inhibit LSD1 demethylase activity.

Amino acid sequences of BHC80 polypeptides, and nucleotide sequences encoding BHC80 polypeptides, are publicly available. See, e.g., 1) GenBank Accession No. NP_{—}057705 for a Homo sapiens BHC80 amino acid sequence; and GenBank NM_{—}016621 for a nucleotide sequence encoding the amino acid sequence set forth in GenBank Accession No. NP_{—}057705; 2) GenBank Accession No. NP_{—}620094 for a Mus musculus BHC80 amino acid sequence; and GenBank NM_{—}138755 for a nucleotide sequence encoding the amino acid sequence set forth in GenBank Accession No. NP_{—}620094; 3) GenBank Accession No. NP00118576.1 for a Gallus gallus BHC80 amino acid sequence; and GenBank NM_{—}001199647 for a nucleotide sequence encoding the amino acid sequence set forth in GenBank Accession No. NP00118576.1; and 4) GenBank Accession No. DAA21793 for a Bos taurus BHC80 amino acid sequence.

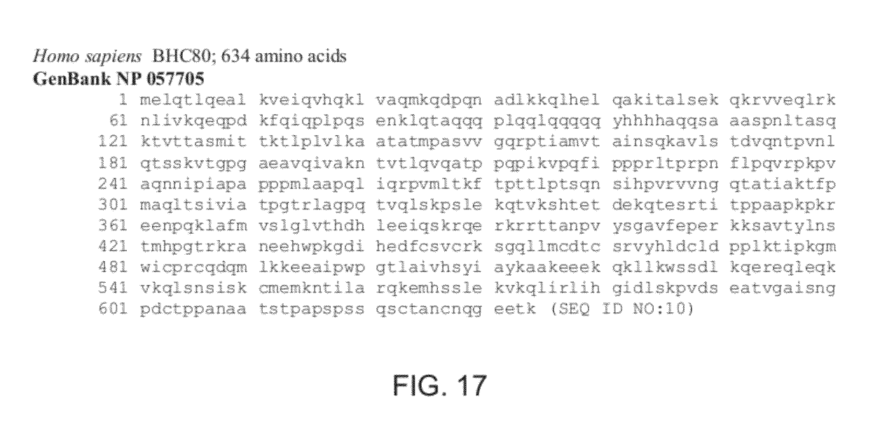

A BHC80 polypeptide can comprise an amino acid sequence having at least about 75%, at least about 80%, at least about 85%, at least about 90%, at least about 95%, at least about 98%, at least about 99%, or 100%, amino acid sequence identity to a contiguous stretch of from about 100 amino acids (aa) to about 200 aa, from about 200 aa to about 300 aa, from about 300 aa to about 400 aa, from about 400 aa to about 500 aa, from about 500 aa to about 600 aa, or from about 600 aa to 634 aa, of the amino acid sequence depicted in FIG. 17 (SEQ ID NO:10).

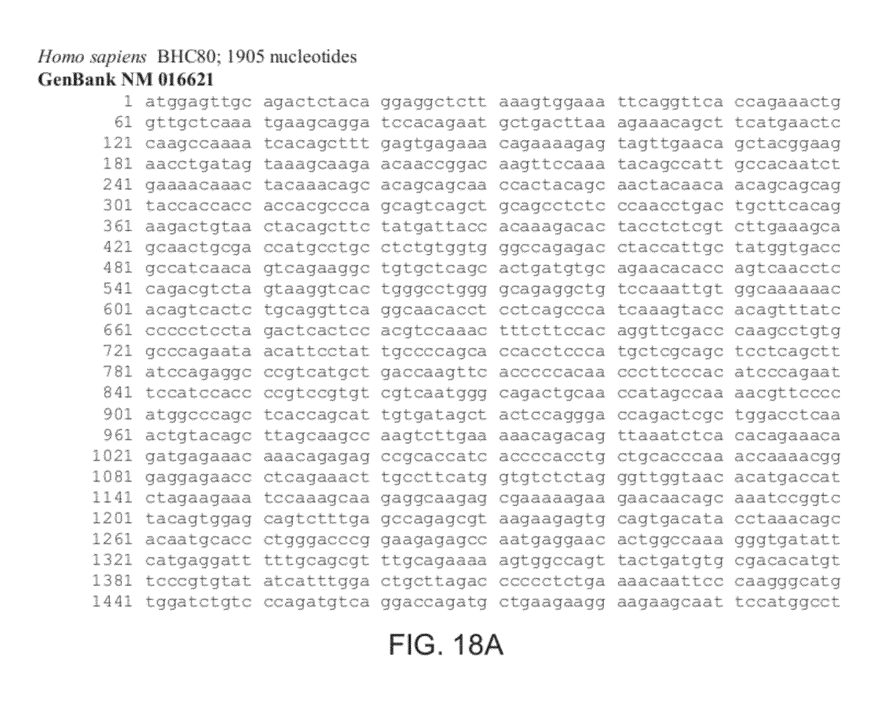

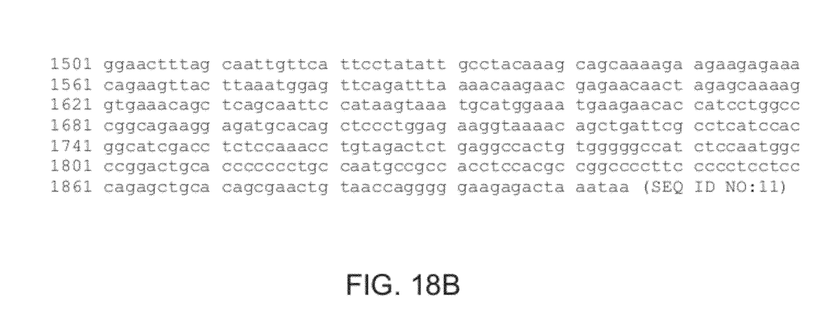

A BHC80 polypeptide can be encoded by a nucleotide sequence having at least about 75%, at least about 80%, at least about 85%, at least about 90%, at least about 95%, at least about 98%, at least about 99%, or 100%, nucleotide sequence identity with a contiguous stretch of from about 300 nucleotides (nt) to about 500 nt, from about 500 nt to about 1000 nt, from about 1000 nt to about 1500 nt, or from about 1500 nt to 1905 nt, of the nucleotide sequence set forth in FIGS. 18A and 18B (SEQ ID NO:11).

A BHC80 nucleic acid (a nucleic acid comprising a nucleotide sequence encoding a BHC80 polypeptide) can comprise a nucleotide sequence having at least about 75%, at least about 80%, at least about 85%, at least about 90%, at least about 95%, at least about 98%, at least about 99%, or 100%, nucleotide sequence identity with a contiguous stretch of from about 300 nucleotides (nt) to about 500 nt, from about 500 nt to about 1000 nt, from about 1000 nt to about 1500 nt, or from about 1500 nt to 1905 nt, of the nucleotide sequence set forth in FIGS. 18A and 18B (SEQ ID NO:11).

Small Molecule Agents that Reduce Enzymatic Activity of LSD1

Small molecule agents that reduce enzymatic activity of LSD1 (also referred to herein as “LSD1 inhibitors”) that are suitable for use in a subject method include monoamine oxidase (MAO) inhibitors that also inhibit LSD1 enzymatic activity; polyamine compounds that inhibit LSD1 enzymatic activity; phenylcyclopropylamine derivatives that inhibit LSD1 enzymatic activity; and the like.

In some embodiments, a suitable agent that inhibits enzymatic activity of an LSD1 polypeptide has an IC₅₀ of less than 50 μM, e.g., an LSD1 inhibitor suitable for use in a subject method has an IC₅₀ of from about 50 μM to about 5 nm, or less than 5 nM. For example, in some embodiments, an LSD1 inhibitor suitable for use in a subject method has an IC₅₀ of from about 50 μM to about 25 μM, from about 25 μM to about 10 μM, from about 10 μM to about 5 μM, from about 5 μM to about 1 μM, from about 1 μM to about 500 nM, from about 500 nM to about 400 nM, from about 400 nM to about 300 nM, from about 300 nM to about 250 nM, from about 250 nM to about 200 nM, from about 200 nM to about 150 nM, from about 150 nM to about 100 nM, from about 100 nM to about 50 nM, from about 50 nM to about 30 nM, from about 30 nM to about 25 nM, from about 25 nM to about 20 nM, from about 20 nM to about 15 nM, from about 15 nM to about 10 nM, from about 10 nM to about 5 nM, or less than about 5 nM.

MAO inhibitors that are suitable for use in a subject method of inhibiting immunodeficiency virus and/or in a subject method of treating an immunodeficiency virus infection include MAO-A-selective inhibitors, MAO-B-selective inhibitors, and MAO non-selective inhibitors. Non-limiting examples of MAO inhibitors include reported inhibitors of the MAO-A isoform, which preferentially deaminates 5-hydroxytryptamine (serotonin) (5-HT) and norepinephrine (NE), and/or the MAO-B isoform, which preferentially deaminates phenylethylamine (PEA) and benzylamine (both MAO-A and MAO-B metabolize Dopamine (DA)). In various embodiments, MAO inhibitors may be irreversible or reversible (e.g., reversible inhibitors of MAO-A (RIMA)), and may have varying potencies against MAO-A and/or MAO-B (e.g., non-selective dual inhibitors or isoform-selective inhibitors).

Non-limiting examples of MAO inhibitors useful in a subject method of inhibiting immunodeficiency virus and/or in a subject method of treating an immunodeficiency virus infection include clorgyline; L-deprenyl; isocarboxazid (Marplan™); ayahuasca; nialamide; iproniazide; iproclozide; moclobemide (Aurorix™; 4-chloro-N-(2-morpholin-4-ylethyl)benzamide); phenelzine (Nardil™; (±)-2-phenylethylhydrazine); tranylcypromine (Parnate™; (±)-trans-2-phenylcyclopropan-1-amine) (the congeneric of phenelzine); toloxatone; levo-deprenyl (Selegiline™); harmala; RIMAs (e.g., moclobemide, described in Da Prada et al., J Pharmacol Exp Ther 248: 400-414 (1989); brofaromine; and befloxatone, described in Curet et al., J Affect Disord 51: 287-303 (1998), lazabemide (Ro 19 6327), described in Ann. Neurol., 40(1): 99-107 (1996), and SL25.1131, described in Aubin et al., J. Pharmacol. Exp. Ther., 310: 1171-1182 (2004)); selegiline hydrochloride (1-deprenyl, ELDEPRYL, ZELAPAR); dimethylselegilene; safinamide; rasagiline (AZILECT); bifemelane; desoxypeganine; harmine (also known as telepathine or banasterine); linezolid (ZYVOX, ZYVOXID); pargyline (EUDATIN, SUPIRDYL); dienolide kavapyrone desmethoxyyangonin; 5-(4-Arylmethoxyphenyl)-2-(2-cyanoethyl)tetrazoles; and the like.

Inhibitors of LSD1 enzymatic activity that are suitable for use in a subject method of inhibiting immunodeficiency virus and/or in a subject method of treating an immunodeficiency virus infection include polyamine compounds as described by Woster et al. in U.S. Publication No. 2007/0208082, which is herein incorporated by reference.

Polyamine inhibitors of LSD1 enzymatic activity that are suitable for use in a subject method of inhibiting immunodeficiency virus and/or in a subject method of treating an immunodeficiency virus infection include a compound of the formula (I):

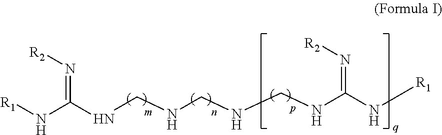

or a salt, solvate, or hydrate thereof, where
n is an integer from 1 to 12;
m and p are independently an integer from 1 to 5;
q is 0 or 1;
each R₁ is independently selected from the group consisting of C₁-C₈ alkyl, C₄-C₁₅ cycloalkyl, C₃-C₁₅ branched alkyl, C₆-C₂₀ aryl, C₆-C₂₀ heteroaryl, C₇-C₂₄ aralkyl, C₇-C₂₄ heteroaralkyl, and

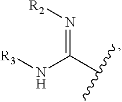

where R₃ is selected from the group consisting of C₁-C₈ alkyl, C₄-C₁₅ cycloalkyl, C₃-C₁₅ branched alkyl, C₆-C₂₀ aryl, C₆-C₂₀ heteroaryl, C₇-C₂₄ aralkyl and C₇-C₂₄ heteroaralkyl; and

each R₂ is independently selected from hydrogen or a C₁-C₈ alkyl.

A suitable polyamine compound is a compound of Formula (I), wherein one or both R₁ is a C₆-C₂₀ aryl, such as a single ring aryl, including without limitation, a phenyl. In one embodiment, the compound is of the formula (I) and each R₁ is phenyl. In one embodiment, q is l, m and p are 3, and n is 4. In another embodiment, q is l, m and p are 3, and n is 7.

A suitable polyamine compound is a compound of Formula (I), where at least one or both R₁ is a C₈-C₁₂ or a C₁-C₈ alkyl, such as a linear alkyl. One or both R₁ may be a C₁-C₈ linear alkyl, such as methyl or ethyl. In one embodiment, each R₁ is methyl. One or both R₁ may comprise or be a C₄-C₁₅ cycloalkyl group, such as a cycloalkyl group containing a linear alkyl group, where the cycloalkyl group is connected to the molecule either via its alkyl or cycloalkyl moiety. For instance, one or both R₁ may be cyclopropylmethyl or cyclohexylmethyl. In one embodiment, one R₁ is cyclopropylmethyl or cyclohexylmethyl and the other R₁ is a linear alkyl group, such as a linear C₁-C₈ unsubstituted alkyl group, including without limitation an ethyl group. In one embodiment, R₁ is a C₃-C₁₅ branched alkyl group such as isopropyl. When R₁ is a C₁-C₈ substituted alkyl, the substituted alkyl may be substituted with any substituent, including a primary, secondary, tertiary or quaternary amine. Accordingly, in one embodiment, R₁ is a C₁-C₈ alkyl group substituted with an amine such that R₁ may be e.g., alkyl-NH₂ or an alkyl-amine-alkyl moiety such as —(CH₂)_{y}NH(CH₂)_{z}CH₃ where y and z are independently an integer from 1 to 8. In one embodiment, R₁ is —(CH₂)₃NH₂.

In one embodiment, the compound is of the formula (I) where one or both R₁ is a C₇-C₂₄ substituted or unsubstituted aralkyl, which in one embodiment is an aralkyl connected to the molecule via its alkyl moiety (e.g., benzyl). In one embodiment, both R₁ are aralkyl moieties wherein the alkyl portion of the moiety is substituted with two aryl groups and the moiety is connected to the molecule via its alkyl group. For instance, in one embodiment one or both R₁ is a C₇-C₂₄ aralkyl wherein the alkyl portion is substituted with two phenyl groups, such as when R₁ is 2,2-diphenylethyl or 2,2-dibenzylethyl. In one embodiment, both R₁ of formula (I) is 2,2-diphenylethyl and n is 1, 2 or 5. In one embodiment, each R₁ of formula (I) is 2,2-diphenylethyl, n is 1, 2 or 5 and m and p are each 1.

In one embodiment, at least one R₁ is hydrogen. When one R₁ is hydrogen, the other R₁ may be any moiety listed above for R₁, including an aryl group such as benzyl. Any of the compounds of formula (I) listed above include compounds where at least one or both of R₂ is hydrogen or a C₁-C₈ substituted or unsubstituted alkyl. In one embodiment, each R₂ is an unsubstituted alkyl such as methyl. In another embodiment, each R₂ is hydrogen. Any of the compounds of formula (I) listed above may be compounds where q is 1 and m and p are the same. Accordingly, the polyaminoguanidines of formula (I) may be symmetric with reference to the polyaminoguanidine core (e.g., excluding R₁). Alternatively, the compounds of formula (I) may be asymmetric, e.g., when q is 0. In one embodiment, m and p are 1. In one embodiment, q is 0. In one embodiment, n is an integer from 1 to 5.

In one embodiment, the compound is a polyaminobiguanide or N-alkylated polyaminobiguanide. An N-alkylated polyaminobiguanide intends a polyaminobiguanide where at least one imine nitrogen of at least one biguanide is alkylated. In one embodiment, the compound is a polyaminobiguanide of the formula (I), or a salt, solvate, or hydrate thereof, where q is 1, and at least one or each R₁ is of the structure:

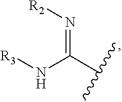

where each R₃ is independently selected from the group consisting of C₁-C₈ alkyl, C₆-C₂₀ aryl, C₆-C₂₀ heteroaryl, C₇-C₂₄ aralkyl, and C₇-C₂₄ heteroaralkyl; and each R₂ is independently hydrogen or a C₁-C₈ alkyl.

In one embodiment, in the polyaminobiguanide compound, at least one or each R₃ is a C₁-C₈ alkyl. For instance, when R₃ is a C₁-C₈ alkyl, the alkyl may be substituted with any substituent, including a primary, secondary, tertiary or quaternary amine. Accordingly, in one embodiment, R₃ is a C₁-C₈ alkyl group substituted with an amine such that R₃ may be e.g., alkyl-NH₂ or an alkyl-amine-alkyl moiety such as —(CH₂)_{y}NH(CH₂)_{z}CH₃ where y and z are independently an integer from 1 to 8. In one embodiment, R₃ is —(CH₂)₃NH₂. R₃ may also be a C₄-C₁₅ cycloalkyl or a C₃-C₁₅ branched alkyl. In one embodiment, at least one or each R₃ is a C₆-C₂₀ aryl. In one embodiment, q is l, m and p are 3, and n is 4. In another embodiment, q is l, m and p are 3, and n is 7.

In one embodiment, the compound is a polyaminobiguanide of formula (I) where at least one R₃ is a C₇-C₂₄ aralkyl, which in one embodiment is an aralkyl connected to the molecule via its alkyl moiety. In one embodiment, each R₃ is an aralkyl moiety where the alkyl portion of the moiety is substituted with one or two aryl groups and the moiety is connected to the molecule via its alkyl moiety. For instance, in one embodiment at least one or each R₃ is an aralkyl where the alkyl portion is substituted with two phenyl or benzyl groups, such as when R₃ is 2,2-diphenylethyl or 2,2-dibenzylethyl. In one embodiment, each R₃ is 2,2-diphenylethyl and n is 1, 2 or 5. In one embodiment, each R₃ is 2,2-diphenylethyl and n is 1, 2 or 5 and m and p are each 1.

Any of the polyaminobiguanide compounds of formula (I) listed above include compounds where at least one or both of R₂ is hydrogen or a C₁-C₈ alkyl. In one embodiment, each R₂ is an unsubstituted alkyl, such as methyl. In another embodiment, each R₂ is a hydrogen.

Any of the polyaminobiguanide compounds of formula (I) listed above include compounds where q is 1 and m and p are the same. Accordingly, the polyaminobiguanides of formula (I) may be symmetric with reference to the polyaminobiguanide core. Alternatively, the compounds of formula (I) may be asymmetric. In one embodiment, m and p are 1. In one embodiment, q is 0. In one embodiment, n is an integer from 1 to 5. In one embodiment, q, m and p are each 1 and n is 1, 2 or 5.

It is understood and clearly conveyed by this disclosure that each R₁, R₂, R₃, m, n, p and q disclosed in reference to formula (I) intends and includes all combinations thereof the same as if each and every combination of R₁, R₂, R₃, m, n, p and q were specifically and individually listed.

Representative compounds of the formula (I) include, e.g.:

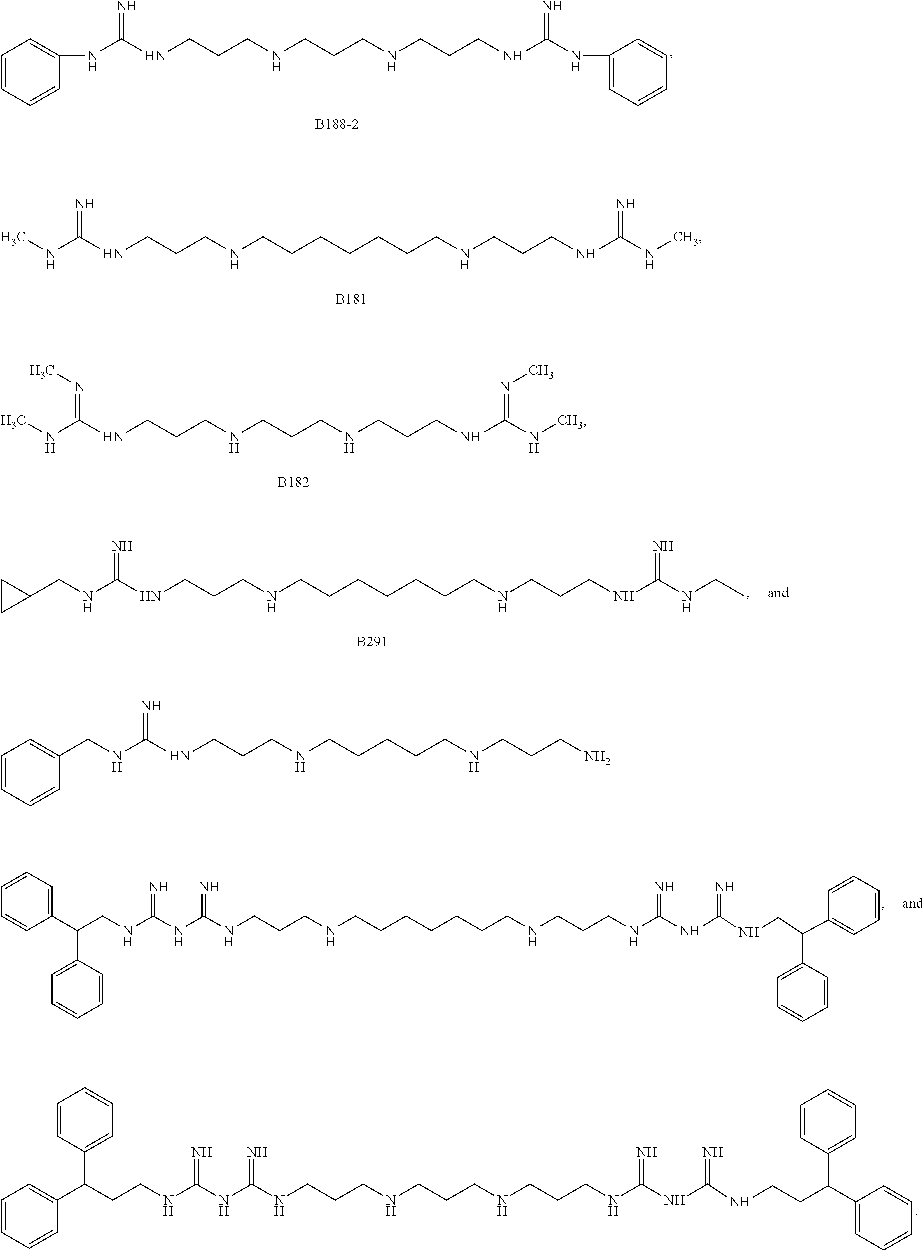

In certain embodiments, the polyamine compound is of the structure of Formula (II):

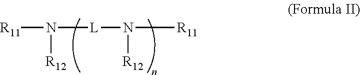

or a salt, solvate or hydrate thereof,

where n is 1, 2 or 3;

each L is independently a linker of from about 2 to 14 carbons in length, for example of about 2, 3, 4, 5, 6, 8, 10, 12 or 14 carbon atoms in length, where the linker backbone atoms may be saturated or unsaturated, usually not more than one, two, three, or four unsaturated atoms will be present in a tether backbone, where each of the backbone atoms may be substituted or unsubstituted (for example with a C₁-C₈ alkyl), where the linker backbone may include a cyclic group (for example, a cyclohex-1,3-diyl group where 3 atoms of the cycle are included in the backbone);

each R₁₂ is independently selected from hydrogen and a C₁-C₈ alkyl; and

each R₁₁ is independently selected from hydrogen, C₂-C₈ alkenyl, C₁-C₈ alkyl or C₃-C₈ branched alkyl (e.g., methyl, ethyl, tert-butyl, isopropyl, pentyl, cyclobutyl, cyclopropylmethyl, 3-methylbutyl, 2-ethylbutyl, 5-NH₂-pent-1-yl, propyl-1-ylmethyl(phenyl)phosphinate, dimethylbicyclo[3.1.1]heptyl)ethyl, 2-(decahydronaphthyl)ethyl and the like), C₆-C₂₀ aryl or heteroaryl, C₁-C₂₄ aralkyl or heteroaralkyl (2-phenylbenzyl, 4-phenylbenzyl, 2-benzylbenzyl, 3-benzylbenzyl, 3,3-diphenylpropyl, 3-(benzoimidazolyl)-propyl, 4-isopropylbenzyl, 4-fluorobenzyl, 4-tert-butylbenzyl, 3-imidazolyl-propyl, 2-phenylethyl and the like), —C(═O)—C₁-C₈ alkyl, —C(═O)—C₁-C₈ alkenyl, —C(═O)—C₁-C₈ alkynyl, an amino-substituted cycloalkyl (e.g., a cycloalkyl group substituted with a primary, secondary, tertiary or quaternary amine, such as 5-NH₂-cycloheptyl, 3-NH₂-cyclopentyl and the like) and a C₂-C₈ alkanoyl (e.g., an alkanoyl substituted with a methyl and an alkylazide group).

In certain embodiments, each L is independently selected from: —CHR₁₃—(CH₂)ₘ—, —CHR₁₃—(CH₂)ₙ—CHR₁₃—, —(CH₂)ₘCHR₁₃—, —CH₂-A-CH₂— and —(CH₂)ₚ—

where:

m is an integer from 1 to 5;

A is (CH₂)ₘ, ethane-1,1-diyl or cyclohex-1,3-diyl;

p is an integer from 2 to 14, such as 1, 2, 3, 4 or 5;

n is an integer from 1 to 12; and

R₁₃ is a C₁-C₈ alkyl.

A substituted aralkyl or heteroaralkyl with reference to formula (II) intends and includes alkanoyl moieties substituted with an aryl or heteroaryl group, i.e., —C(═O)-aryl, —C(═O)-aralkyl, —C(═O)-heteroaryl, and —C(═O)-heteroaralkyl. In one embodiment, the alkyl portion of the aralkyl or heteroaralkyl moiety is connected to the molecule via its alkyl moiety. For instance at least one or both of R₁₁ may be an aralkyl moiety such as 2-phenylbenzyl, 4-phenylbenzyl, 3,3,-diphenylpropyl, 2-(2-phenylethyl)benzyl, 2-methyl-3-phenylbenzyl, 2-napthylethyl, 4-(pyrenyl)butyl, 2-(3-methylnapthyl)ethyl, 2-(1,2-dihydroacenaphth-4-yl)ethyl and the like. In another embodiment, at least one or both of R₁₁ may be a heteroaralkyl moiety such as 3-(benzoimidazolyl)propanoyl, 1-(benzoimidazolyl)methanoyl, 2-(benzoimidazolyl)ethanoyl, 2-(benzoimidazolyl)ethyl and the like.

In certain embodiments, the compound of formula (II) comprises at least one moiety selected from the group consisting of t-butyl, isopropyl, 2-ethylbutyl, 1-methylpropyl, 1-methylbutyl, 3-butenyl, isopent-2-enyl, 2-methylpropan-3-olyl, ethylthiyl, phenylthiyl, propynoyl, 1-methyl-1H-pyrrole-2-yl; trifluoromethyl, cyclopropanecarbaldehyde, halo-substituted phenyl, nitro-substituted phenyl, alkyl-substituted phenyl, 2,4,6-trimethylbenzyl, halo-5-substituted phenyl (such as para-(F₃S)-phenyl, azido and 2-methylbutyl.

In certain embodiments, in formula (II), each R₁₁ is independently selected from hydrogen, n-butyl, ethyl, cyclohexylmethyl, cyclopentylmethyl, cyclopropylmethyl, cycloheptylmethyl, cyclohexyleth-2-yl, and benzyl.

In certain embodiments, the polyamine compound is of the structure of Formula (II), where n is 3, such that the compound is of the structure of Formula

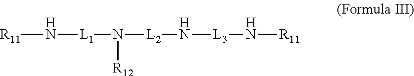

where L₁, L₂ and L₃ are independently selected from —CHR₁₃—(CH₂)ₘ—, —CHR₁₃—(CH₂)ₙ—CHR₁₃—, —(CH₂)ₘ—CHR₁₃—, —CH₂-A-CH₂— and —(CH₂)ₚ—

where m, A, p, n and R₁₃ are as defined above.

In certain embodiments, the polyamine compound is of the structure of Formula (III) where:
L₁ is —CHR₁₃—(CH₂)ₘ—;
L₂ is —CHR₁₃—(CH₂)ₙ—CHR₁₃—; and
L₃ is —(CH₂)ₘ—CHR₁₃—;
where R₁₁, R₁₂, R₁₃, m and n are as defined above.

In certain embodiments, the polyamine compound is of the structure of Formula (III) where:
L₁, L₂ and L₃ are independently —CH₂-A-CH₂—; and
R₁₂ is hydrogen;
where R₁₁ and A are as defined above. In particular embodiments, at least one of an A and an R₁₁ comprises an alkenyl moiety.

In certain embodiments, the polyamine compound is of the structure of Formula (III) where:
L₁, L₂ and L₃ are independently —(CH₂)ₚ— where p is as defined above; and
R₁₂ is hydrogen. In particular embodiments, for L₁ and L₃, p is an integer from 3 to 7, and for L₃ p is an integer from 3 to 14.

In certain embodiments, the polyamine compound is of the structure of Formula (III) where:
L₁, and L₃ are independently —(CH₂)ₚ—;
L₂ is —CH₂-A-CH₂—; and
R₁₂ is hydrogen;
where R₁₂, p and A are as defined above. In particular embodiments, for L₁ and L₃, p is an integer from 2 to 6, and for L₃ A is (CH₂)^{x} where x is an integer from 1 to 5, or cyclohex-1,3-diyl.

In certain embodiments, the polyamine compound is of the structure of Formula (II), where n is 2, such that the compound is of the structure of Formula (IV):

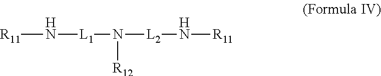

where L₁ and L₂ are independently selected from —CHR₁₃—(CH₂)ₘ—CHR₁₃—(CH₂)ₙ—CHR₁₃—, —(CH₂)ₙ, CHR₁₃—, —CH₂-A-CH₂— and —(CH₂)ₚ—

where m, A, p, n, and R₁₃ are as defined above.

In certain embodiments, the polyamine compound is of the structure of Formula (IV) where:
L₁ is —(CH₂)ₚ—; and
L₂ is —(CH₂)ₘ—CHR₁₃—;
where R₁₃, m and p are as defined above. In particular embodiments, for L₁ p is an integer from 3 to 10, and for L₂ n is an integer from 2 to 9.

In certain embodiments, the polyamine compound is of the structure of Formula (IV) where:
L₁ and L₂ are —(CH₂)ₚ—;
where p is as defined above. In particular embodiments, p is an integer from 3 to 7.

In certain embodiments, the polyamine compound is of the structure of Formula (II), where n is 1, such that the compound is of the structure of Formula (V):

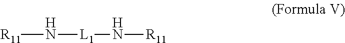

where L₁ is —(CH₂)ₚ— where p is as defined above. In particular embodiments, p is an integer from 2 to 6.

In particular embodiments, in Formula (V), one R₁₁ is an amino-substituted cycloalkyl (e.g., a cycloalkyl group substituted with a primary, secondary, tertiary or quaternary amine) or a C₂-C₈ alkanoyl (which alkanoyl may be substituted with one or more substituents such as a methyl or an alkylazide group); and the other R₁₁ is a C₁-C₈ alkyl or a C₇-C₂₄ aralkyl.

Representative compounds of the formula (II) include, e.g.:

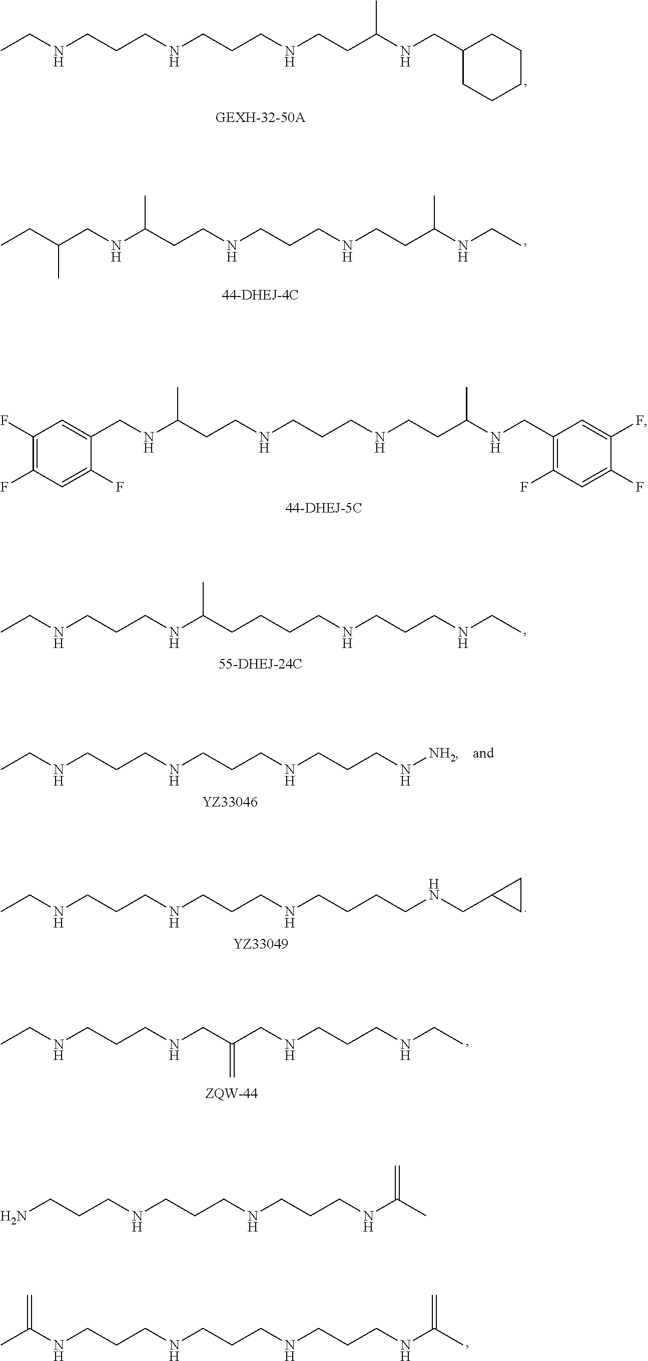

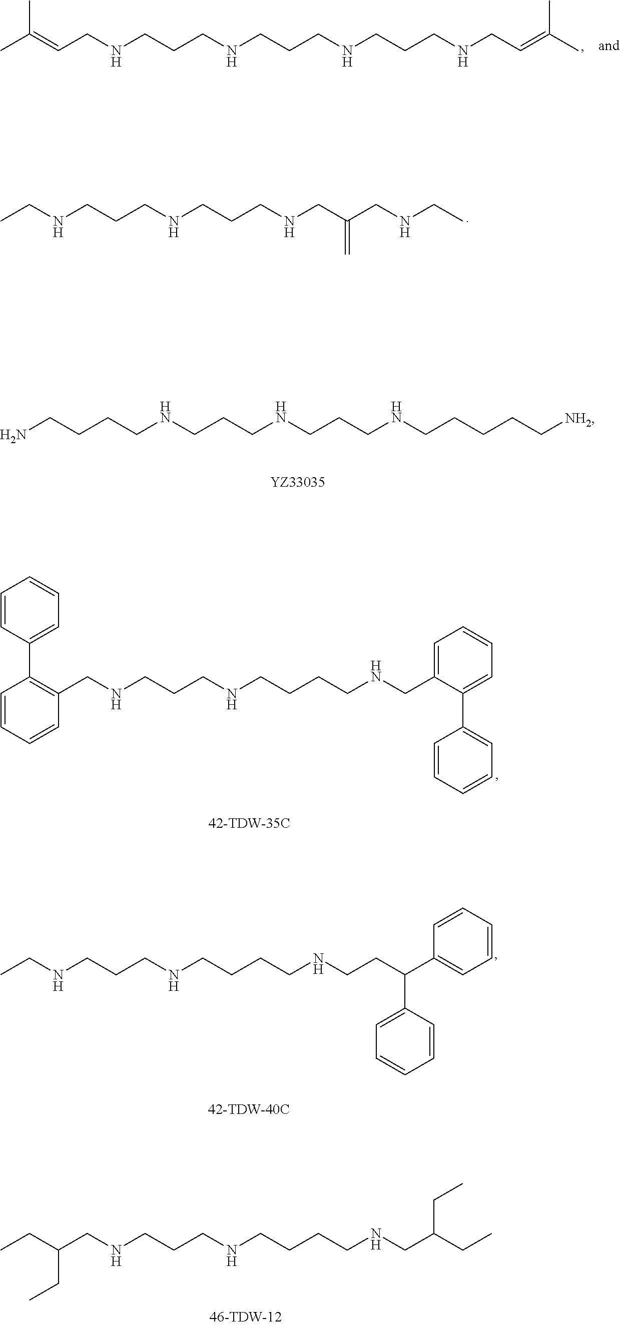

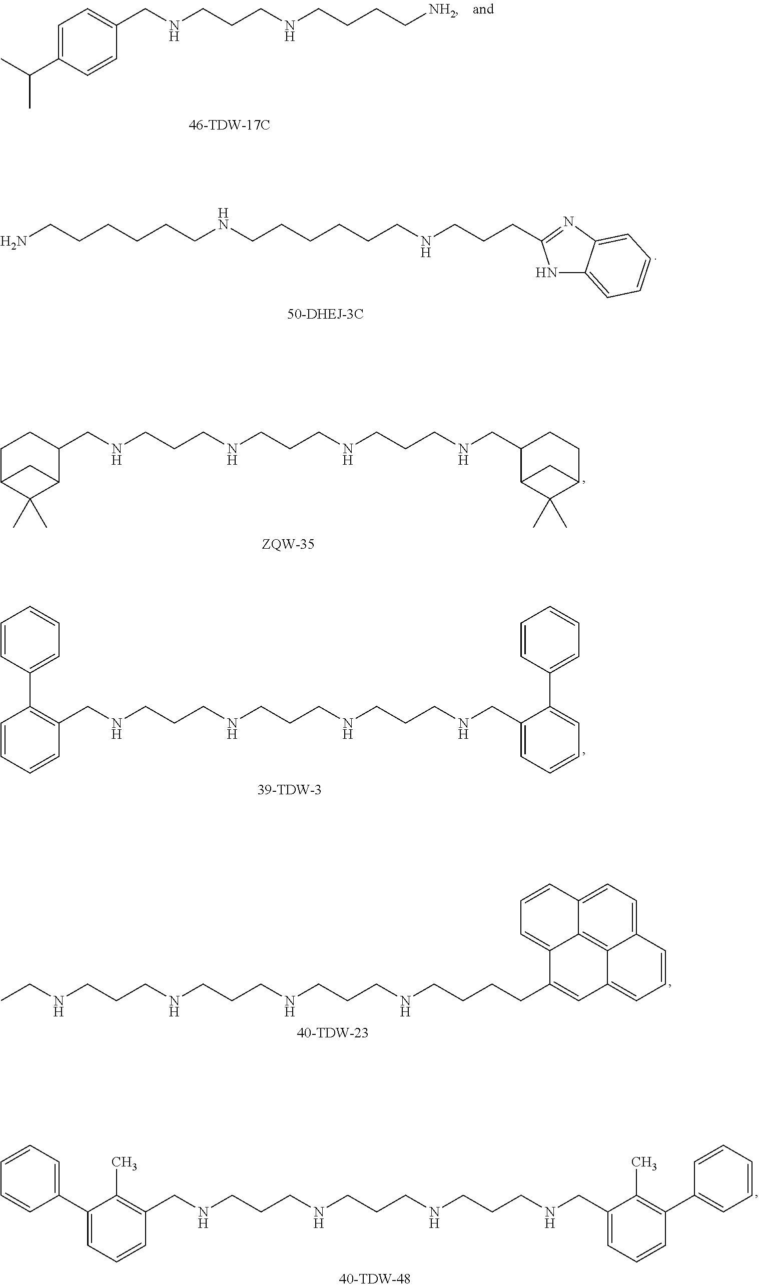

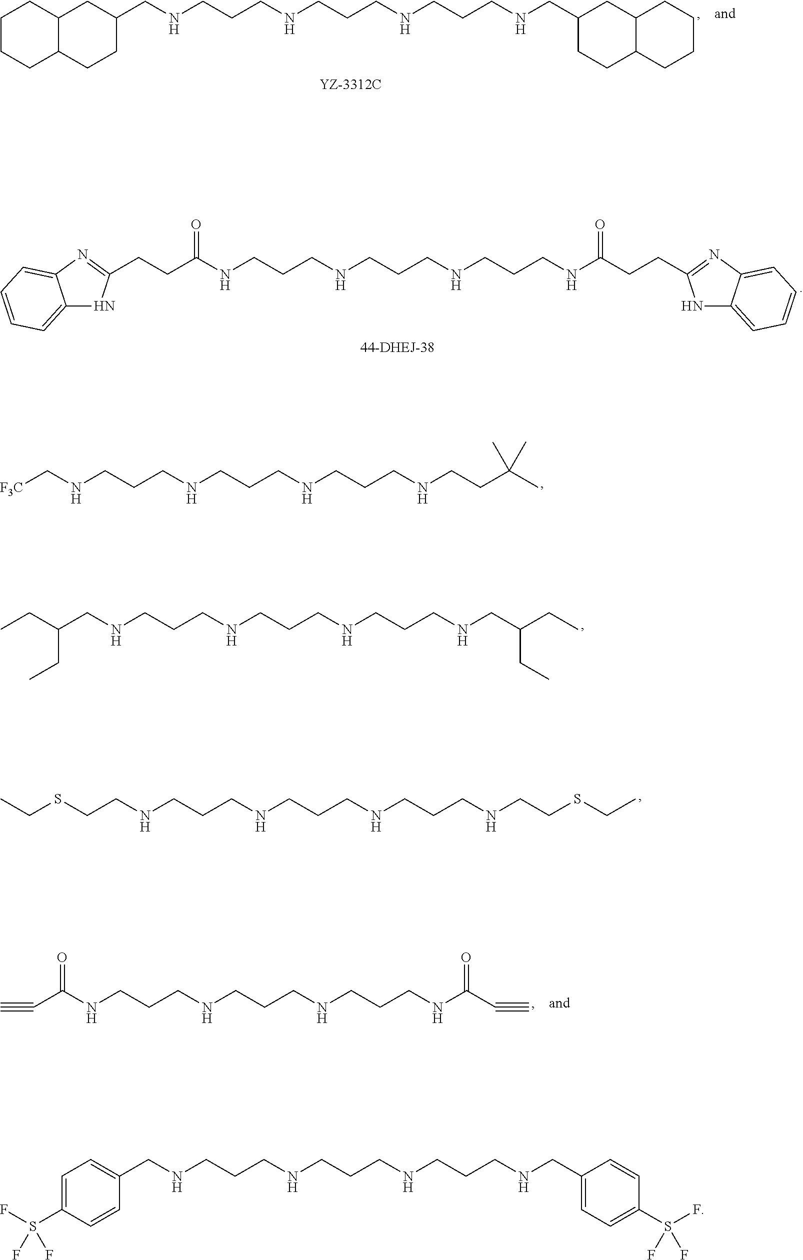

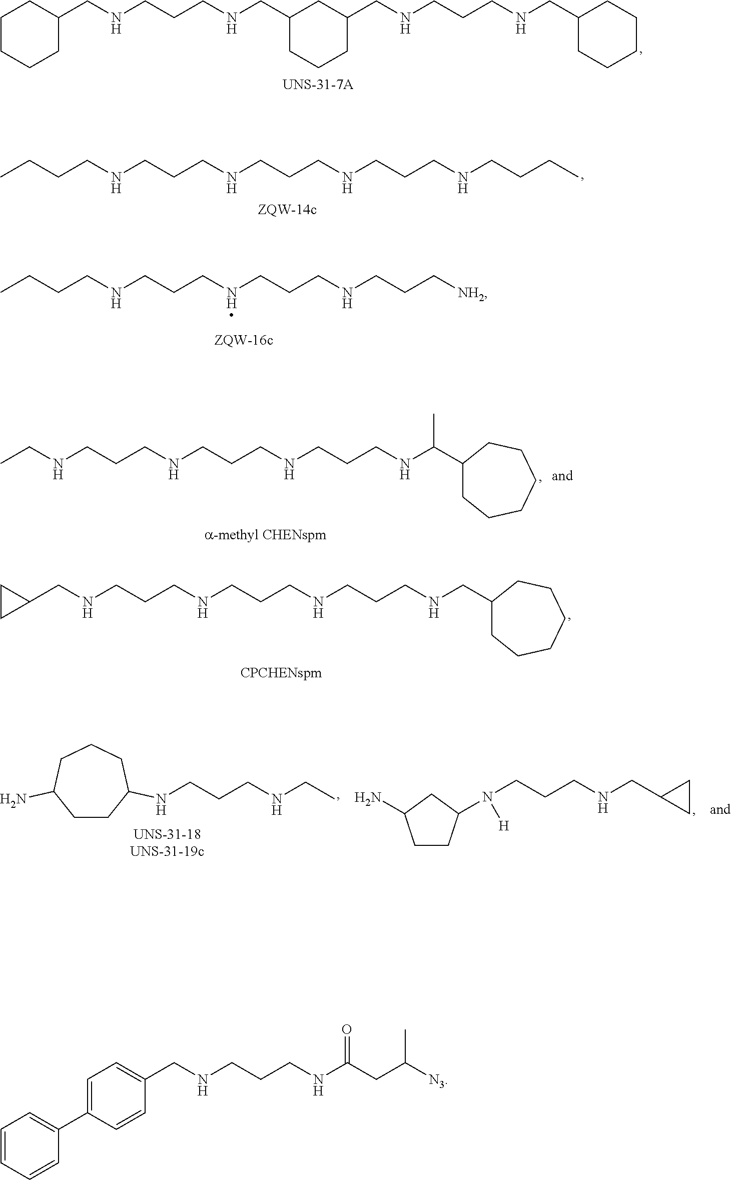

Phenylcyclopropylamine derivatives that are inhibitors of LSD1 enzymatic activity and that are suitable for use in a subject method of inhibiting immunodeficiency virus and/or in a subject method of treating an immunodeficiency virus infection include a compound of the formula:

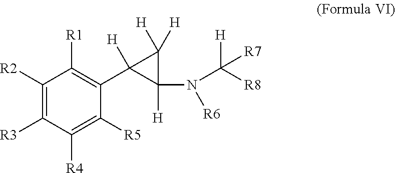

wherein:

each of R1-R5 is independently selected from H, halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanate, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;

R6 is H or alkyl;

R7 is H, alkyl, or cycloalkyl;

R8 is an -L-heterocyclyl wherein the ring or ring system of the -L-heterocyclyl has from 0 to 3 substituents selected from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanate, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido; or

R8 is -L-aryl wherein the ring or ring system of the -L-aryl has from 1 to 3 substituents selected from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanate, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;

where each L is independently selected from —(CH₂)ₙ—(CH₂)ₙ—, —(CH₂)ₙNH(CH₂)ₙ—, —(CH₂)ₙO(CH₂)ₙ—, and —(CH₂)ₙS(CH₂)ₙ—, and where each n is independently chosen from 0, 1, 2, and 3;

or a pharmaceutically acceptable salt thereof.

In some cases, L is a covalent bond. In some cases, R6 and R7 are hydro. In some cases, one of R1-R5 is selected from -L-aryl, -L-heterocyclyl, and -L-carbocyclyl.

In some embodiments of the compound of Formula VI, the substituent or substituents on the R8 ring or ring system is/are selected from hydroxyl, halo, alkyl, alkoxy, cycloalkoxy, haloalkyl, haloalkoxy, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃ alkyl), —S(═O)₂NH₂, —S(O)₂NH₂, —S(O)₂N(C₁₋₃ alkyl)₂, —S(═O)₂NH(C₁₋₃ alkyl), —CN, —NH₂, and —NO₂.

In certain embodiments, a compound of the invention is of formula (VI) where:

each R1-R5 is optionally substituted and independently chosen from —H, halo, alkyl, alkoxy, cycloalkoxy, haloalkyl, haloalkoxy, -L-aryl, -L-heteroaryl, -L-heterocyclyl, -L-carbocyclyl, acylamino, acyloxy, alkylthio, cycloalkylthio, alkynyl, amino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N— thiocarbamyl, and C-amido;

R6 is chosen from —H and alkyl;

R7 is chosen from —H, alkyl, and cycloalkyl;

R8 is chosen from —C(═O)NRxRy and —C(═O)Rz;

Rx when present is chosen from —H, alkyl, alkynyl, alkenyl, -L-carbocyclyl, -L-aryl, and -L-heterocyclyl, all of which are optionally substituted (except —H);

Ry when present is chosen from —H, alkyl, alkynyl, alkenyl, -L-carbocyclyl, -L-aryl, and -L-heterocyclyl, all of which are optionally substituted (except —H), where Rx and Ry may be cyclically linked;

Rz when present is chosen from —H, alkoxy, -L-carbocyclyl, -L-heterocyclyl, -L-aryl, wherein the aryl, heterocyclyl, or carbocyclyl are optionally substituted; each L is a linker that links the main scaffold of Formula I to a carbocyclyl, heterocyclyl, or aryl group, wherein the hydrocarbon portion of the linker -L- is saturated, partially saturated, or unsaturated, and is independently chosen from a saturated parent group having a formula of —(CH₂)ₙ—(CH₂)ₙ—, —(CH₂)ₙC(═O)(CH₂)—, —(CH₂)ₙC(═O)NH(CH₂)ₙ—, —(CH₂)ₙNHC(O)O(CH₂)ₙ—, —(CH₂)ₙNHC(═O)NH(CH₂)ₙ—, —(CH₂)ₙNHC(═S)S(CH₂)ₙ—, —(CH₂)ₙOC(═O)S(CH₂)ₙ—, —(CH₂)ₙNH(CH₂)ₙ—, —(CH₂)ₙ—O—(CH₂)ₙ—, —(CH₂)ₙS(CH₂)ₙ—, and —(CH₂)ₙNHC(═S)NH(CH₂)ₙ—, where each n is independently chosen from 0, 1, 2, 3, 4, 5, 6, 7, and 8. According to this embodiment, optionally substituted refers to zero or 1 to 4 optional substituents independently chosen from acylamino, acyloxy, alkenyl, alkoxy, cycloalkoxy, alkyl, alkylthio, cycloalkylthio, alkynyl, amino, aryl, arylalkyl, arylalkenyl, arylalkynyl, arylalkoxy, aryloxy, arylthio, heteroarylthio, carbocyclyl, cyano, cyanato, halo, haloalkyl, haloaryl, hydroxyl, heteroaryl, heteroaryloxy, heterocyclyl, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido. In a more specific aspect of this embodiment, the optional substituents are 1 or 2 optional substituents chosen from halo, alkyl, aryl, and arylalkyl.

In certain embodiments, in formula V(I), R8 is —CORz, such that the compound is of the following structure:

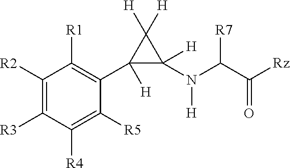

where:
R1-R7 are described above; and
Rz is -L-heterocyclyl which is optionally substituted with from 1-4 optional substituents independently chosen from acylamino, acyloxy, alkenyl, alkoxy, cycloalkoxy, alkyl, alkylthio, cycloalkylthio, alkynyl, amino, aryl, arylalkyl, arylalkenyl, arylalkynyl, arylalkoxy, aryloxy, arylthio, heteroarylthio, carbocyclyl, cyano, cyanato, halo, haloalkyl, haloaryl, hydroxyl, heteroaryl, heteroaryloxy, heterocyclyl, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido, and wherein said -L- is independently chosen from —(CH₂)ₙ—(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, and 3.

In a specific aspect of this embodiment, each L is independently chosen from —(CH₂)ₙ—(CH₂)ₙ— and —(CH₂)ₙ—O—(CH₂)ₙ where each n is independently chosen from 0, 1, 2, and 3. In a more specific aspect of this embodiment, each L is chosen from a bond, —CH₂—, —CH₂CH₂—, —OCH₂—, —OCH₂CH₂—, —CH₂OCH₂—, —CH₂CH₂CH₂—, —OCH₂CH₂CH₂—, and —CH₂OCH₂CH₂—. In an even more specific aspect, each L is chosen from a bond, —CH₂—, —CH₂CH₂—, OCH₂—, and —CH₂CH₂CH₂—. In yet an even more specific aspect, L is chosen from a bond and —CH₂—.

Exemplary compounds of Formula VI include:

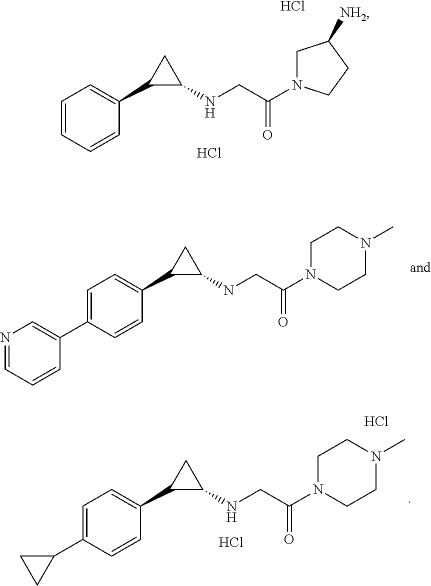

Exemplary compounds of Formula VI include:
N-cyclopropyl-2-{[(trans)-2-phenylcyclopropyl]amino}acetamide;
2-{[(trans)-2-phenylcyclopropyl]amino acetamide;
N-cyclopropyl-2-{[(trans)-2-phenylcyclopropyl]amino}propanamide;
2-{[(trans)-2-phenylcyclopropyl]amino}-N-prop-2-ynylacetamide;
N-isopropyl-2-{[(trans)-2-phenylcyclopropyl]amino}acetamide;
N-(tert-butyl)-2-{[(trans)-2-phenylcyclopropyl]amino}acetamide;
N-(2-morpholin-4-yl-2-oxoethyl)-N-[(trans)-2-phenylcyclopropyl]amine;
2-{[(trans)-2-phenylcyclopropyl]amino}propanamide;
methyl 2-{[(trans)-2-phenylcyclopropyl]amino}propanoate;
N-cyclopropyl-2-{methyl[(trans)-2-phenylcyclopropyl]amino}acetamide;
2-{methyl[(trans)-2-phenylcyclopropyl]amino}acetamide;
N-methyl-trans-2-(phenylcyclopropylamino)propanamide;
1-(4-methylpiperazin-1-yl)-2-((trans)-2-phenylcyclopropylamino)ethanone;
1-(4-ethylpiperazin-1-yl)-2-((trans)-2-phenylcyclopropylamino)ethanone;
1-(4-benzylpiperazin-1-yl)-2-((trans)-2-phenylcyclopropylamino)-ethanone;
2-((trans)-2-phenylcyclopropylamino)-1-(4-phenylpiperazin-1-yl)ethanone;
2-((trans)-2-(4-(benzyloxy)phenyl)cyclopropylamino)-1-(4-methylpiperazin-1-yl)ethanone;
2-((trans)-2-(4-(benzyloxy)phenyl)cyclopropylamino)-N-cyclopropylacetamide;
2-((trans)-2-(4-(3-fluorobenzyloxy)phenyl)cyclopropylamino)-1-(4-methylpiperazin-1-yl)ethanone;
2-((trans)-2-(4-(3-chlorobenzyloxy)phenyl)cyclopropylamino)-1-(4-methylpiperazin-1-yl)ethanone;
2-((trans)-2-(biphenyl-4-yl)cyclopropylamino)-1-(4-methylpiperazin-1-yl)ethanone;
1-(4-methylpiperazin-1-yl)-2-((trans)-2-(4-phenethoxyphenyl)cyclopropylamino)ethanone;
2-((trans)-2-(4-(4-fluorobenzyloxy)phenyl)cyclopropylamino)-1-(4-methylpiperazin-1-yl)ethanone;
2-((trans)-2-(4-(biphenyl-4-ylmethoxy)phenyl)cyclopropylamino)-1-(4-methylpiperazin-1-yl)ethanone;
(trans)-N-(4-fluorobenzyl)-2-phenylcyclopropanamine;
(trans)-N-(4-fluorobenzyl)-2-phenylcyclopropanaminiurn;
4-(((trans)-2-phenylcyclopropylamino)methyl)benzonitrile;
(trans)-N-(4-cyanobenzyl)-2-phenylcyclopropanaminium;
(trans)-2-phenyl-N-(4-(trifluoromethyl)benzyl)cyclopropanamine;
(trans)-2-phenyl-N-(4-(trifluoromethyl)benzyl)cyclopropanaminium;
(trans)-2-phenyl-N-(pyridin-2-ylmethyl)cyclopropanamine;
(trans)-2-phenyl-N-(pyridin-3-ylmethyl)cyclopropanamine;
(trans)-2-phenyl-N-(pyridin-4-ylmethyl)cyclopropanamine;
(trans)-N-((6-methylpyridin-2-yl)methyl)-2-phenylcyclopropanamine;
(trans)-2-phenyl-N-(thiazol-2-ylmethyl)cyclopropanamine;
(trans)-2-phenyl-N-(thiophen-2-ylmethyl)cyclopropanamine;
(trans)-N-((3-bromothiophen-2-yl)methyl)-2-phenylcyclopropanamine;
(trans)-N-((4-bromothiophen-2-yl)methyl)-2-phenylcyclopropanamine;
(trans)-N-(3,4-dichlorobenzyl)-2-phenylcyclopropanamine;
(trans)-N-(3-fluorobenzyl)-2-phenylcyclopropanaminium;
(trans)-N-(2-fluorobenzyl)-2-phenylcyclopropanamine;
(trans)-2-phenyl-N-(quinolin-4-ylmethyl)cyclopropanaraine;
(trans)-N-(3-methoxybenzyl)-2-phenylcyclopropanamine;
(trans)-2-phenyl-N-((6-(trifluoromethyl)pyridin-3-yl)methyl)cyclopropanamine;
(trans)-N-((6-chloropyridin-3-yl)methyl)-2-phenylcyclopropanamine;
(trans)-N-((4-methylpyridin-2-yl)methyl)-2-phenylcyclopropanamine;
(trans)-N-((6-methoxypyridin-2-yl)methyl)-2-phenylcyclopropanamine;
2-(((trans)-2-phenylcyclopropylamino)methyl)pyridin-3-ol;
(trans)-N-((6-bromopyridin-2-yl)methyl)-2-phenylcyclopropanamine;
4-(((trans)-2-(4(benzyloxy)phenyl)cyclopropylamino)methyl)benzonitrile;
(trans)-N-(4-(benzyloxy)benzyl)-2-phenylcyclopropanamine;
(trans)-N-benzyl-2-(4-(benzyloxy)phenyl)cyclopropanamine;
(trans)-2-(4-(benzyloxy)phenyl)-N-(4-methoxybenzyl)cyclopropanamine;
(trans)-2-(4-(benzyloxy)phenyl)-N-(4-fluorobenzyl)cyclopropanamine;
(trans)-2-phenyl-N-(quinolin-2-ylmethyl)cyclopropanamine;
(trans)-2-phenyl-N-((5-(trifluoromethyl)pyridin-2-yl)methyl)cyclopropanamine;
(trans)-N-((3-fluoropyridin-2-yl)methyl)-2-phenylcyclopropanamine;
(trans)-2-phenyl-N-(quinolin-3-ylmethyl)cyclopropanamine;
(trans)-N-((6-methoxypyridin-3-yl)methyl)-2-phenylcyclopropanamine;
(trans)-N-((5-methoxypyridin-3-yl)methyl)-2-phenylcyclopropanamine;
(trans)-N-((2-methoxypyridin-3-yl)methyl)-2-phenylcyclopropanamine;
(trans)-N-((3H-indol-3-yl)methyl)-2-phenylcyclopropanamine;
3-(((trans)-2-phenylcyclopropylamino)methyl)benzonitrile;
(trans)-N-(2-methoxybenzyl)-2-phenylcyclopropanamine;
3-(((trans)-2-phenylcyclopropylamino)methyl)pyridin-2-amine;
(trans)-N-((2-chloropyridin-3-yl)methyl)-2-phenylcyclopropanamine;
(trans)-N-(3,4-dimethoxybenzyl)-2-phenylcyclopropanamine;
(trans)-N-((2,3-dihydrobenzofuran-5-yl)methyl)-2-phenylcyclopropanamine;
(trans)-N-(benzo[d][1,3]dioxol-5-ylmethyl)-2-phenylcyclopropanamine;
(trans)-N-((2,3-dihydrobenzo[b][1,4]dioxin-6-yl)methyl)-2-phenylcyclopropanamine;
(trans)-N-(2,6-difluoro-4-methoxybenzyl)-2-phenylcyclopropanamine;
(trans)-2-phenyl-N-(4-(trifluoromethoxy)benzyl)cyclopropanamine;
(trans)-N-(5-fluoro-2-methoxybenzyl)-2-phenylcyclopropanamine;
(trans)-N-(2-fluoro-4-methoxybenzyl)-2-phenylcyclopropanamine;
(trans)-N-((4-methoxynaphthalen-1-yl)methyl)-2-phenylcyclopropanamine;
(trans)-N-(2-fluoro-6-methoxybenzyl)-2-phenylcyclopropanamine;
(trans)-N-((2-methoxynaphthalen-1-yl)methyl)-2-phenylcyclopropanamine;
(trans)-N-((4,7-dimethoxynaphthalen-1-yl)methyl)-2-phenylcyclopropanamine;
(trans)-N-(4-methoxy-3-methylbenzyl)-2-phenylcyclopropanamine;
(trans)-N-(3-chloro-4-methoxybenzyl)-2-phenylcyclopropanamine;
(trans)-N-(3-fluoro-4-methoxybenzyl)-2-phenylcyclopropanamine;
(trans)-N-(4-methoxy-2-methylbenzyl)-2-phenylcyclopropanamine;
(trans)-N-((3,4-dihydro-2H-benzo[b][1,4]dioxepin-6-yl)methyl)-2-phenylcyclopropanamine;
(trans)-N-((3,4-dihydro-2H-benzo[b][1,4]dioxepin-7-yl)methyl)-2-phenylcyclopropanamine;
(trans)-N-((2,2-dimethylchroman-6-yl)methyl)-2-phenylcyclopropanamine;
(trans)-N-(4-methoxy-2,3-dimethylbenzyl)-2-phenylcyclopropanamine;
(trans)-N-(4-methoxy-2,5-dimethylbenzyl)-2-phenylcyclopropanamine;
(trans)-N-(2-fluoro-4,5-dimethoxybenzyl)-2-phenylcyclopropanamine;
(trans)-N-(3-chloro-4,5-dimethoxybenzyl)-2-phenylcyclopropanamine;
(trans)-N-(2-chloro-3,4-dimethoxybenzyl)-2-phenylcyclopropanamine;
(trans)-N-(2,4-dimethoxy-6-methylbenzyl)-2-phenylcyclopropanamine;
(trans)-N-(2,5-dimethoxybenzyl)-2-phenylcyclopropanamine;
(trans)-N-(2,3-dimethoxybenzyl)-2-phenylcyclopropanamine;
(trans)-N-(2-chloro-3-methoxybenzyl)-2-phenylcyclopropanamine;
(trans)-N-((1H-indol-5-yl)methyl)-2-phenylcyclopropanamine;
(trans)-2-(4-(benzyloxy)phenyl)-N-(pyridin-2-ylmethyl)cyclopropanamine;
(trans)-2-(4-(benzyloxy)phenyl)-N-(2-methoxybenzyl)cyclopropanamine;
(trans)-N-(1-(4-methoxyphenyl)ethyl)-2-phenylcyclopropanaraine;
(trans)-N-(1-(3,4-dimethoxyphenyl)ethyl)-2-phenylcyclopropanamine;
(trans)-N-(1-(2,3-dihydrobenzo[b][1,4]dioxin-6-yl)ethyl)-2-phenylcyclopropanamine;
(trans)-N-(1-(5-fluoro-2-methoxyphenyl)ethyl)-2-phenylcyclopropanamine;
(trans)-N-(1-(3,4-dimethoxyphenyl)propan-2-yl)-2-phenylcyclopropanamine;
(trans)-N-((3-methyl-1,2,4-oxadiazol-5-yl)methyl)-2-phenylcyclopropanamine;

and pharmaceutically acceptable salts thereof.

BHC80 Polypeptides that Reduce LSD1 Activity

In some instances, an agent that inhibits LSD1 enzymatic activity is a BHC80 polypeptide. The BHC80 polypeptide can be a full-length BHC80 polypeptide, or a fragment of a BHC80 polypeptide that inhibits LSD1 enzymatic activity.

A suitable BHC80 polypeptide can have a length of from about 25 amino acids (aa) to about 50 aa, from about 50 aa to about 100 aa, from about 100 aa to about 200 aa, from about 200 aa to about 300 aa, from about 300 aa to about 400 aa, from about 400 aa to about 500 aa, from about 500 aa to about 600 aa, or from about 600 aa to 634 aa, and can comprise an amino acid sequence having at least about 75%, at least about 80%, at least about 85%, at least about 90%, at least about 95%, at least about 98%, at least about 99%, or 100%, amino acid sequence identity to a contiguous stretch of from about 25 amino acids (aa) to about 50 aa, from about 50 aa to about 100 aa, from about 100 aa to about 200 aa, from about 200 aa to about 300 aa, from about 300 aa to about 400 aa, from about 400 aa to about 500 aa, from about 500 aa to about 600 aa, or from about 600 aa to 634 aa, of the amino acid sequence depicted in FIG. 17.

A BHC80 polypeptide can be introduced into a cell by delivering a polypeptide per se, or by introducing into the cell a BHC80 nucleic acid comprising a nucleotide sequence encoding a BHC80 polypeptide. A BHC nucleic acid can comprise a nucleotide sequence having at least about 75%, at least about 80%, at least about 85%, at least about 90%, at least about 95%, at least about 98%, at least about 99%, or 100%, nucleotide sequence identity with a contiguous stretch of from about 300 nucleotides (nt) to about 500 nt, from about 500 nt to about 1000 nt, from about 1000 nt to about 1500 nt, or from about 1500 nt to 1905 nt, of the nucleotide sequence set forth in FIGS. 18A and 18B.

The BHC80 nucleic acid can be a recombinant expression vector. The BHC80-encoding nucleotide sequence can be operably linked to a transcriptional control element(s), e.g., a promoter, in the expression vector. Suitable vectors include, e.g., recombinant retroviruses, lentiviruses, and adenoviruses; retroviral expression vectors, lentiviral expression vectors, nucleic acid expression vectors, and plasmid expression vectors. In some cases, the expression vector is integrated into the genome of a cell. In other cases, the expression vector persists in an episomal state in a cell.

Suitable expression vectors include, but are not limited to, viral vectors (e.g. viral vectors based on vaccinia virus; poliovirus; adenovirus (see, e.g., Li et al., Invest Opthalmol V is Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol V is Sci 38:2857 2863, 1997; Jomary et al., Gene Ther 4:683 690, 1997, Rolling et al., Hum Gene Ther 10:641 648, 1999; Ali et al., Hum Mol Genet. 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Interfering Nucleic Acid Agents that Reduce the Level of LSD1 in a Cell

Agents that reduce the level of LSD1 in a cell include nucleic acid agents (“inhibitory nucleic acids”) that reduce the level of active LSD1 in a cell. Suitable agents that reduce the level of LSD1 activity in a cell (e.g., that reduce the total amount of LSD1 in a cell) include interfering nucleic acids, e.g., interfering RNA molecules. In one embodiment, reduction of LSD1 levels is accomplished through RNA interference (RNAi) by contacting a cell with a small nucleic acid molecule, such as a short interfering nucleic acid (siNA), a short interfering RNA (siRNA), a double-stranded RNA (dsRNA), a micro-RNA (miRNA), or a short hairpin RNA (shRNA) molecule, or modulation of expression of a small interfering RNA (siRNA) so as to provide for decreased levels of LSD1.

LSD1-specific interfering nucleic acids can be designed based on the nucleotide sequence of an LSD1-encoding nucleotide sequence. For example, in some embodiments, an LSD1-encoding nucleotide sequence as set forth in SEQ ID NO:2 or SEQ ID NO:4 is used to design an interfering nucleic acid.

Agents that reduce the level of LSD1 in a cell include nucleic acid agents (“inhibitory nucleic acids”) that reduce expression of CoREST in the cell, which in turn reduces the level of active LSD1 polypeptide in a cell. Suitable agents that reduce expression of CoREST in a cell include interfering nucleic acids, e.g., interfering RNA molecules. In one embodiment, reduction of CoREST expression is accomplished through RNA interference (RNAi) by contacting a cell with a small nucleic acid molecule, such as a short interfering nucleic acid (siNA), a short interfering RNA (siRNA), a double-stranded RNA (dsRNA), a micro-RNA (miRNA), or a short hairpin RNA (shRNA) molecule, or modulation of expression of a small interfering RNA (siRNA) so as to provide for decreased expression of CoREST, thereby decreasing the level of LSD1 polypeptide.

CoREST-specific interfering nucleic acids can be designed based on the nucleotide sequence of a CoREST-encoding nucleotide sequence. For example, in some embodiments, a CoREST-encoding nucleotide sequence as set forth in FIG. 16 is used to design an interfering nucleic acid.

The term “short interfering nucleic acid,” “siNA,” “short interfering RNA,” “siRNA,” “short interfering nucleic acid molecule,” “short interfering oligonucleotide molecule,” or “chemically-modified short interfering nucleic acid molecule” as used herein refers to any nucleic acid molecule capable of inhibiting or down regulating gene expression, for example by mediating RNA interference “RNAi” or gene silencing in a sequence-specific manner. Design of RNAi molecules when given a target gene is routine in the art. See also US 2005/0282188 (which is incorporated herein by reference) as well as references cited therein. See, e.g., Pushparaj et al. Clin Exp Pharmacol Physiol. 2006 May-June; 33(5-6):504-10; Lutzelberger et al. Handb Exp Pharmacol. 2006; (173):243-59; Aronin et al. Gene Ther. 2006 March; 13(6):509-16; Xie et al. Drug Discov Today. 2006 January; 11(1-2):67-73; Grunweller et al. Curr Med. Chem. 2005; 12(26):3143-61; and Pekaraik et al. Brain Res Bull. 2005 Dec. 15; 68(1-2):115-20. Epub 2005 Sep. 9.

Methods for design and production of siRNAs to a desired target are known in the art, and their application to LSD1 genes or CoREST genes for the purposes disclosed herein will be readily apparent to the ordinarily skilled artisan, as are methods of production of siRNAs having modifications (e.g., chemical modifications) to provide for, e.g., enhanced stability, bioavailability, and other properties to enhance use as therapeutics. In addition, methods for formulation and delivery of siRNAs to a subject are also well known in the art. See, e.g., US 2005/0282188; US 2005/0239731; US 2005/0234232; US 2005/0176018; US 2005/0059817; US 2005/0020525; US 2004/0192626; US 2003/0073640; US 2002/0150936; US 2002/0142980; and US2002/0120129, each of which is incorporated herein by reference.

Publicly available tools to facilitate design of siRNAs are available in the art. See, e.g., DEQOR: Design and Quality Control of RNAi (available on the internet at cluster-1.mpi-cbg.de/Degor/deqor.html). See also, Henschel et al. Nucleic Acids Res. 2004 Jul. 1; 32(Web Server issue):W113-20. DEQOR is a web-based program which uses a scoring system based on state-of-the-art parameters for siRNA design to evaluate the inhibitory potency of siRNAs. DEQOR, therefore, can help to predict (i) regions in a gene that show high silencing capacity based on the base pair composition and (ii) siRNAs with high silencing potential for chemical synthesis. In addition, each siRNA arising from the input query is evaluated for possible cross-silencing activities by performing BLAST searches against the transcriptome or genome of a selected organism. DEQOR can therefore predict the probability that an mRNA fragment will cross-react with other genes in the cell and helps researchers to design experiments to test the specificity of siRNAs or chemically designed siRNAs.

A suitable siRNA or shRNA sequence for reducing LSD1 expression includes, e.g., 5′-GAAGGCTCTTCTAGCAATA-3′ (SEQ ID NO:36).

siNA molecules can be of any of a variety of forms. For example the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. siNA can also be assembled from two separate oligonucleotides, where one strand is the sense strand and the other is the antisense strand, wherein the antisense and sense strands are self-complementary. In this embodiment, each strand generally comprises nucleotide sequence that is complementary to nucleotide sequence in the other strand; such as where the antisense strand and sense strand form a duplex or double stranded structure, for example wherein the double stranded region is about 15 base pairs to about 30 base pairs, e.g., about 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 nucleotides to about 25 or more nucleotides of the siNA molecule are complementary to the target nucleic acid or a portion thereof).

Alternatively, the siNA can be assembled from a single oligonucleotide, where the self-complementary sense and antisense regions of the siNA are linked by a nucleic acid-based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof.

The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (e.g., where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate.

In certain embodiments, the siNA molecule contains separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der Waals interactions, hydrophobic interactions, and/or stacking interactions. In certain embodiments, the siNA molecules comprise nucleotide sequence that is complementary to nucleotide sequence of a target gene. In another embodiment, the siNA molecule interacts with nucleotide sequence of a target gene in a manner that causes inhibition of expression of the target gene.

As used herein, siNA molecules need not be limited to those molecules containing only RNA, but further encompasses chemically-modified nucleotides and non-nucleotides. In certain embodiments, the short interfering nucleic acid molecules lack 2′-hydroxy (2′-OH) containing nucleotides. siNAs do not necessarily require the presence of nucleotides having a 2′-hydroxy group for mediating RNAi and as such, siNA molecules optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules can also be referred to as short interfering modified oligonucleotides “siMON.”

As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siNA molecules can be used to epigenetically silence a target gene at the post-transcriptional level, the pre-transcriptional level, or both the post-transcriptional and pre-transcriptional levels. In a non-limiting example, epigenetic regulation of gene expression by siNA molecules can result from siNA mediated modification of chromatin structure or methylation pattern to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

siNA molecules contemplated herein can comprise a duplex forming oligonucleotide (DFO) see, e.g., WO 05/019453; and US 2005/0233329, which are incorporated herein by reference). siNA molecules also contemplated herein include multifunctional siNA, (see, e.g., WO 05/019453 and US 2004/0249178). The multifunctional siNA can comprise sequence targeting, for example, two regions of LSD1, or two regions of CoREST.

siNA molecules contemplated herein can comprise an asymmetric hairpin or asymmetric duplex. By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a loop region comprising about 4 to about 12 (e.g., about 4, 5, 6, 7, 8, 9, 10, 11, or 12) nucleotides, and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant a siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a sense region having about 3 to about 25 (e.g., about 3, 4, 5, 6, 7, 8, 9, 10, 11, 12, 13, 14, 15, 16, 17, 18, 19, 20, 21, 22, 23, 24, or 25) nucleotides that are complementary to the antisense region.

Stability and/or half-life of siRNAs can be improved through chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein, describing various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Eamshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; each of which are hereby incorporated in their totality by reference herein). In view of such teachings, similar modifications can be used as described herein to modify the siNA nucleic acid molecules of disclosed herein so long as the ability of siNA to promote RNAi is cells is not significantly inhibited.

Short interfering nucleic acid (siNA) molecules having chemical modifications that maintain or enhance activity are contemplated herein. Such a nucleic acid is also generally more resistant to nucleases than an unmodified nucleic acid. Accordingly, the in vitro and/or in vivo activity should not be significantly lowered. Nucleic acid molecules delivered exogenously are generally selected to be stable within cells at least for a period sufficient for transcription and/or translation of the target RNA to occur and to provide for modulation of production of the encoded mRNA and/or polypeptide so as to facilitate reduction of the level of the target gene product.

Production of RNA and DNA molecules can be accomplished synthetically and can provide for introduction of nucleotide modifications to provide for enhanced nuclease stability. (see, e.g., Wincott et al., 1995, Nucleic Acids Res. 23, 2677; Caruthers et al., 1992, Methods in Enzymology 211, 3-19, incorporated by reference herein. In one embodiment, nucleic acid molecules include one or more (e.g., about 1, 2, 3, 4, 5, 6, 7, 8, 9, 10, or more) G-clamp nucleotides, which are modified cytosine analogs which confer the ability to hydrogen bond both Watson-Crick and Hoogsteen faces of a complementary guanine within a duplex, and can provide for enhanced affinity and specificity to nucleic acid targets (see, e.g., Lin et al. 1998, J. Am. Chem. Soc., 120, 8531-8532). In another example, nucleic acid molecules can include one or more (e.g., about 1, 2, 3, 4, 5, 6, 7, 8, 9, 10, or more) LNA “locked nucleic acid” nucleotides such as a 2′,4′-C methylene bicyclo nucleotide (see, e.g., Wengel et al., WO 00/66604 and WO 99/14226).

siNA molecules can be provided as conjugates and/or complexes, e.g., to facilitate delivery of siNA molecules into a cell. Exemplary conjugates and/or complexes include those composed of an siNA and a small molecule, lipid, cholesterol, phospholipid, nucleoside, antibody, toxin, negatively charged polymer (e.g., protein, peptide, hormone, carbohydrate, polyethylene glycol, or polyamine). In general, the transporters described are designed to be used either individually or as part of a multi-component system, with or without degradable linkers. These compounds can improve delivery and/or localization of nucleic acid molecules into cells in the presence or absence of serum (see, e.g., U.S. Pat. No. 5,854,038). Conjugates of the molecules described herein can be attached to biologically active molecules via linkers that are biodegradable, such as biodegradable nucleic acid linker molecules.

### Dominant Negative CoREST Polypeptide Agents

As noted above, in some cases, an agent that reduces the level of enzymatically active LSD1 in a cell is an agent that inhibits binding of CoREST to LSD1 in a cell. In some cases, the agent is a CoREST polypeptide that binds an LSD1 polypeptide, but does not enhance enzymatic activity of the LSD1 polypeptide. Such a CoREST polypeptide can be considered a dominant negative polypeptide. Suitable dominant negative CoREST polypeptides include, but are not limited to, a polypeptide having a length of from about 5 amino acids to about 275 amino acids, where the dominant negative CoREST polypeptide comprises an amino acid sequence having at least about 75%, at least about 80%, at least about 85%, at least about 90%, at least about 95%, at least about 98%, at least about 99%, or 100%, amino acid sequence identity to a contiguous stretch of from about 5 amino acids to about 275 amino acids of amino acids 105-381 of the CoREST amino acid sequence set forth in FIG. 15 (SEQ ID NO:8).

For example, a suitable dominant negative CoREST polypeptide has a length of from about 5 amino acids to 88 amino acids (e.g., from about 5 amino acids (aa) to about 10 aa, from about 10 aa to about 15 aa, from about 15 aa to about 20 aa, from about 20 aa to about 25 aa, from about 25 aa to about 30 aa, from about 30 aa to about 40 aa, from about 40 aa to about 50 aa, from about 50 aa to about 60 aa, from about 60 aa to about 70 aa, from about 70 aa to about 80 aa, or from about 80 aa to 88 aa), and comprises an amino acid sequence having at least about 75%, at least about 80%, at least about 85%, at least about 90%, at least about 95%, at least about 98%, at least about 99%, or 100%, amino acid sequence identity to a contiguous stretch of from about 5 amino acids to 88 amino acids (e.g., from about 10 aa, from about 10 aa to about 15 aa, from about 15 aa to about 20 aa, from about 20 aa to about 25 aa, from about 25 aa to about 30 aa, from about 30 aa to about 40 aa, from about 40 aa to about 50 aa, from about 50 aa to about 60 aa, from about 60 aa to about 70 aa, from about 70 aa to about 80 aa, or from about 80 aa to 88 aa; e.g., to a contiguous stretch of amino acids of the same length as the dominant negative CoREST polypeptide) of the following sequence: vkkekhst qaknrakrkp pkgmflsqed veaysanata ttvlrqldm elvsykrqiq nikqtnsalk ekldggiepy rlpeviqkcn a (SEQ ID NO:37).

As non-limiting examples, a dominant negative CoREST polypeptide can be of the following amino acid sequences:

| (SEQ ID NO: 38) | |
|---|---|
| 1) | qaknrakrkp pkgmflsqed veavsanata; |
| (SEQ ID NO: 39) | |
| 2) | ttvlrqldm elvsvkrqiq nikqtnsalk; |
| (SEQ ID NO: 40) | |
| 3) | elvsvkrqiq nikqtnsalk ekldggiepy; |
| (SEQ ID NO: 41) | |
| 4) | nikqtnsalk ekldggiepy rlpeviqkcn; |
| (SEQ ID NO: 42) | |
| 5) | vkkekhst qaknrakrkp pkgmflsqed; |
| (SEQ ID NO: 43) | |
| 6) | qaknrakrkp pkgmflsqed veavsanata ttvlrqldm elvsvkrqiq nikqtnsalk ekldggiepy; |
| (SEQ ID NO: 44) | |
| 7) | pkgmflsqed veavsanata ttvlrqldm elvsvkrqiq nikqtnsalk ekldggiepy rlpeviqkcn; |
| (SEQ ID NO: 45) | |
| 8) | pkgmflsqed veavsanata ttvlrqldm elvsvkrqiq nikqtnsalk ekldggiepy; |
| (SEQ ID NO: 46) | |
| 9) | veavsanata ttvlrqldm elvsvkrqiq nikqtnsalk ekldggiepy; |
| | and |
| (SEQ ID NO: 37) | |
| 10) | vkkekhst qaknrakrkp pkgmflsqed veavsanata ttvlrqldm elvsvkrqiq nikqtnsalk |
| | ekldggiepy rlpeviqkcn a. |

A dominant negative CoREST polypeptide can comprises one or more heterologous amino acid sequences. For example, a dominant negative CoREST polypeptide can comprise a Protein Transduction Domain (PTD). “Protein Transduction Domain” or PTD refers to a polypeptide, polynucleotide, carbohydrate, or organic or inorganic compound that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to the amino terminus of a polypeptide. In some embodiments, a PTD is covalently linked to the carboxyl terminus of a polypeptide.

Exemplary polypeptide PTD include, but are not limited to, a minimal undecapeptide protein transduction domain (corresponding to residues 47-57 of HIV-1 Tat comprising YGRKKRRQRRR; SEQ ID NO:47); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:48); Transportan GWTLNSAGYLLGKINLKALAALAKKIL (SEQ ID NO:49); KALAWEAKLAKALAKALAKHLAKALAKALKCEA (SEQ ID NO:50); and RQIKIWFQNRRMKWKK (SEQ ID NO:51). Exemplary PTDs include but are not limited to, YGRKKRRQRRR (SEQ ID NO:47), RKKRRQRRR (SEQ ID NO:52); an arginine homopolymer of from 3 arginine residues to 50 arginine residues; Exemplary PTD domain amino acid sequences include, but are not limited to, any of the following: YGRKKRRQRRR (SEQ ID NO:47); RKKRRQRR (SEQ ID NO:53); YARAAARQARA (SEQ ID NO:54); THRLPRRRRRR (SEQ ID NO:55); and GGRRARRRRRR (SEQ ID NO:56).

A dominant negative CoREST polypeptide-fusion protein can include, e.g., from N-terminal to C-terminal: 1) PTD-dominant negative CoREST; or 2) dominant negative CoREST-PTD. A PTD can also be inserted within the dominant negative CoREST polypeptide.

### Histone Deacetylase Inhibitors

In some cases, and agent that reduces LSD1-mediated demethylation of a Tat polypeptide in a cell is a histone deacetylase (HDAC) inhibitor, e.g., a histone deacetylase-1 (HDAC1) or a histone deacetylase-2 (HDAC2) inhibitor.

Class I HDACs include HDAC 1, 2, 3, and 8. See, e.g., GenBank NP_{—}004955 (HDAC1); GenBank NP_{—}001518 (HDAC2); GenBank NP_{—}003874 (HDAC3); and GenBank NP_{—}060956 (HDAC8).

Class II HDACs include HDAC 4, 5, 6, 7, 9, and 10. See, e.g., GenBank NP_{—}006028 (HDAC 4); GenBank NP_{—}631944 (HDAC5); GenBank NP_{—}006035 (HDAC6); GenBank NP_{—}057680 (HDAC7); GenBank NP_{—}478056 (HDAC9); and GenBank NP_{—}114408 (HDAC10). See also, e.g., Yang and Grégoire (2005) Mol. Cell. Biol. 25:2873.

Class III HDACs include Sirt1, Sirt2, Sirt3, Sirt4, Sirt5, Sirt6, and Sirt7. See, e.g., Blander and Guarente (2004) Ann. Rev. Biochem. 73:417; and Buck et al. (2004) J. Leukocyte Biol. 75:939.

HDAC inhibitors are known in the art, and any of a variety of HDAC inhibitors can be used. In some cases, the HDAC inhibitor inhibits all Class I HDACs, but does not substantially inhibit any Class II HDAC or any Class III HDAC. In some cases, the HDAC inhibitor specifically inhibits HDAC1 (and does not substantially inhibit other HDAC polypeptides, e.g., does not substantially inhibit HDAC 2, 3, 4, 5, 6, 7, 8, 9, or 10, or any Class III HDAC). In some cases, the HDAC inhibitor specifically inhibits HDAC2 (and does not substantially inhibit other HDAC polypeptides, e.g., does not substantially inhibit HDAC 1, 3, 4, 5, 6, 7, 8, 9, or 10, or any Class III HDAC). In some cases, the HDAC inhibitor inhibits both HDAC1 and HDAC2, but does not substantially inhibit other HDAC polypeptides, e.g., does not substantially inhibit HDAC 3, 4, 5, 6, 7, 8, 9, or 10, or any Class III HDAC.

Examples of HDAC inhibitors include trichostatin A (TSA) ((R,2E,4E)-7-(4-(dimethylamino)phenyl)-N-hydroxy-4,6-dimethyl-7-oxohepta-2,4-dienamide); suberoylanilide hydroxamic acid (SAHA); sulfonamides such as oxamflatin ((E)-N-hydroxy-5-(3-(phenylsulfonamido)phenyl)pent-2-en-4-ynamide); and belinostat (PXD101) ((E)-N-hydroxy-3-(4-(N-phenylsulfamoyl)phenypacrylamide). Other hydroxamic-acid-sulfonamide inhibitors of histone deacetylase are described in: Lavoie et al. (2001) Bioorg. Med. Chem. Lett. 11:2847-50; Bouchain et al. (2003) J. Med. Chem. 846:820-830; Bouchain et al. (2003) Curr. Med. Chem. 10:2359-2372; Marson et al. (2004) Bioorg. Med. Chem. Lett. 14:2477-2481; Finn et al. (2005) Helv. Chim. Acta 88:1630-1657; WO2002030879; WO2003082288; WO20050011661; WO2005108367; WO2006123121; WO2006017214; WO2006017215; and US2005/0234033. Other structural classes of histone deacetylase inhibitors include short chain fatty acids, cyclic peptides, and benzamides. Acharya et al. (2005) Mol. Pharmacol. 68:917-932.

Further examples of HDAC inhibitors include those disclosed in, e.g., Dokmanovic et al. (2007) Mol. Cancer. Res. 5:981; U.S. Pat. No. 7,642,275; U.S. Pat. No. 7,683,185; U.S. Pat. No. 7,732,475; U.S. Pat. No. 7,737,184; U.S. Pat. No. 7,741,494; U.S. Pat. No. 7,772,245; U.S. Pat. No. 7,795,304; U.S. Pat. No. 7,799,825; U.S. Pat. No. 7,803,800; U.S. Pat. No. 7,842,727; U.S. Pat. No. 7,842,835; U.S. Patent Publication No. 2010/0317739; U.S. Patent Publication No. 2010/0311794; U.S. Patent Publication No. 2010/0310500; U.S. Patent Publication No. 2010/0292320; and U.S. Patent Publication No. 2010/0291003. In some cases, a given HDAC inhibitor or class of HDAC inhibitors is specifically excluded. For example, in some cases, TSA is specifically excluded.

Treatment Methods; Administering an Agent that Reduces LSD1 Enzymatic Activity and/or that Reduces LSD1 Levels and/or that Reduces LSD1-Mediated Demethylation of Tat

The present disclosure provides methods for treating an immunodeficiency virus infection in an individual, the methods generally involving administering to the individual an effective amount of an agent that inhibits enzymatic activity of an LSD1 polypeptide and/or that reduces the level of an LSD1 polypeptide in a cell and/or that inhibits LSD1-mediated demethylation of a methylated Tat polypeptide in a cell (e.g., an immunodeficiency virus-infected cell) in the individual. An inhibitor of LSD1 enzymatic activity reduces the level of immunodeficiency virus in a cell infected with immunodeficiency virus, and can be used to treat an immunodeficiency virus infection in an individual. Likewise, an agent that reduces the level of an LSD1 polypeptide in a cell reduces the level of immunodeficiency virus in a cell infected with immunodeficiency virus, and can be used to treat an immunodeficiency virus infection in an individual. Similarly, an agent that inhibits LSD1-mediated demethylation of a methylated Tat polypeptide in a cell reduces the level of immunodeficiency virus in a cell infected with immunodeficiency virus, and can be used to treat an immunodeficiency virus infection in an individual.

Agents that reduce LSD1 enzymatic activity include: 1) small molecule agents that are LSD1 inhibitors; and 2) BHC80 polypeptides.

Small molecule agents that reduce enzymatic activity of LSD1 (also referred to herein as “LSD1 inhibitors”), and that are suitable for use in a subject treatment method, include monoamine oxidase (MAO) inhibitors that also inhibit LSD1 enzymatic activity; polyamine compounds that inhibit LSD1 enzymatic activity; phenylcyclopropylamine derivatives that inhibit LSD1 enzymatic activity; and the like. Such agents are described hereinabove and in, e.g., U.S. Patent Publication No. 2007/0208082; WO 2010/043721, and WO 2010/084160.

Other agents that reduce enzymatic activity of LSD1, and that are suitable for use in a subject treatment method, include BHC80 polypeptides, as described above.

Agents that reduce the level of LSD1, e.g., the level of enzymatically active LSD1, in a cell, and that are suitable for use in a subject treatment method, include: 1) inhibitory nucleic acid agents that specifically reduce LSD1 expression, as described above; 2) inhibitory nucleic acid agents that reduce CoREST expression, as described above; and 3) dominant negative CoREST polypeptides.

Agents that inhibit LSD1-mediated demethylation of methylated Tat include HDAC inhibitors.

In some embodiments, an “effective amount” of an agent that inhibits enzymatic activity of an LSD1 polypeptide and/or that reduces the level of an LSD1 polypeptide and/or that inhibits LSD1-mediated demethylation of methylated Tat is an amount that, when administered to an individual in one or more doses, in monotherapy or in combination therapy, is effective to reduce immunodeficiency virus load in the individual by at least about 10%, at least about 20%, at least about 25%, at least about 30%, at least about 40%, at least about 50%, at least about 60%, at least about 70%, at least about 80%, or greater than 80%, compared to the immunodeficiency virus load in the individual in the absence of treatment with the agent.

In some embodiments, an “effective amount” of an agent that inhibits enzymatic activity of an LSD1 polypeptide and/or that reduces the level of an LSD1 polypeptide and/or that inhibits LSD1-mediated demethylation of methylated Tat is an amount that, when administered to an individual in one or more doses, in monotherapy or in combination therapy, is effective to increase the number of CD4⁺ T cells in the individual by at least about 20%, at least about 25%, at least about 30%, at least about 40%, at least about 50%, at least about 60%, at least about 70%, at least about 80%, at least about 2-fold, at least about 2.5-fold, at least about 3-fold, at least about 5-fold, at least about 10-fold, or greater than 10-fold, compared to the number of CD4⁺ T cells in the individual in the absence of treatment with the agent.

Any of a variety of methods can be used to determine whether a treatment method is effective. For example, methods of determining whether a subject method and/or a given LSD1 inhibitor is effective in reducing immunodeficiency virus (e.g., HIV) viral load, and/or treating an immunodeficiency virus (e.g., HIV) infection, are any known test for indicia of immunodeficiency virus (e.g., HIV) infection, including, but not limited to, measuring viral load, e.g., by measuring the amount of immunodeficiency virus (e.g., HIV) in a biological sample, e.g., using a polymerase chain reaction (PCR) with primers specific for an immunodeficiency virus (e.g., HIV) polynucleotide sequence; detecting and/or measuring a polypeptide encoded by an immunodeficiency virus (e.g., HIV), e.g., p24, gp120, reverse transcriptase, using, e.g., an immunological assay such as an enzyme-linked immunosorbent assay (ELISA) with an antibody specific for the polypeptide; and measuring the CD4⁺ T cell count in the individual.

Methods of assaying an HIV infection (or any indicia associated with an HIV infection) are known in the art, and have been described in numerous publications such as HIV Protocols (Methods in Molecular Medicine, 17) N. L. Michael and J. H. Kim, eds. (1999) Humana Press.

### Methods of Reactivating Latent HIV

The present disclosure provides methods of reactivating latent HIV integrated into the genome of a cell comprising an HIV genome integrated into the genome of a cell (e.g., an HIV-infected cell). The methods generally involve contacting an HIV-infected cell in which HIV is latent with an agent that increases LSD1 enzymatic activity in the cell. Latently infected cells contain replication-competent integrated HIV-1 genomes that are blocked at the transcriptional level, resulting in the absence of viral protein expression.

Agents that increase the level of LSD1 in a cell include: a nucleic acid comprising a nucleotide sequence encoding an LSD1 polypeptide; and an LSD1 polypeptide. Thus, a subject method for reactivating latent HIV integrated into the genome of an HIV-infected cell can comprise contacting the HIV-infected cell with an LSD1 polypeptide or with a nucleic acid comprising a nucleotide sequence encoding an LSD1 polypeptide.

Suitable LSD1 polypeptides are described above. In some embodiments, the LSD1 polypeptide comprises a protein transduction domain.

“Protein Transduction Domain” or PTD refers to a polypeptide, polynucleotide, carbohydrate, or organic or inorganic compound that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to the amino terminus of an LSD1 polypeptide. In some embodiments, a PTD is covalently linked to the carboxyl terminus of an LSD1 polypeptide.

Exemplary protein transduction domains include but are not limited to a minimal undecapeptide protein transduction domain (corresponding to residues 47-57 of HIV-1 Tat comprising YGRKKRRQRRR; SEQ ID NO:47); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:48); Transportan GWTLNSAGYLLGKINLKALAALAKKIL (SEQ ID NO:49); KALAWEAKLAKALAKALAKHLAKALAKALKCEA (SEQ ID NO:50); and RQIKIWFQNRRMKWKK (SEQ ID NO:51). Exemplary PTDs include but are not limited to, YGRKKRRQRRR (SEQ ID NO:47), RKKRRQRRR (SEQ ID NO:52); an arginine homopolymer of from 3 arginine residues to 50 arginine residues; Exemplary PTD domain amino acid sequences include, but are not limited to, any of the following: YGRKKRRQRRR (SEQ ID NO:47); RKKRRQRR (SEQ ID NO:53); YARAAARQARA (SEQ ID NO:54); THRLPRRRRRR (SEQ ID NO:55); and GGRRARRRRRR (SEQ ID NO:56).

A suitable nucleic acid includes a nucleic acid comprising a nucleotide sequence having at least about 75%, at least about 80%, at least about 85%, at least about 90%, at least about 95%, at least about 98%, at least about 99%, or 100%, nucleotide sequence identity to a contiguous stretch of from about 2400 nucleotides to about 2500 nucleotides, or from 2500 nucleotides to 2559 nucleotides, of the nucleotide sequence depicted in FIGS. 9A and 9B and set forth in SEQ ID NO:2. A suitable nucleic acid includes a nucleic acid comprising a nucleotide sequence having at least about 75%, at least about 80%, at least about 85%, at least about 90%, at least about 95%, at least about 98%, at least about 99%, or 100%, nucleotide sequence identity to a contiguous stretch of from about 2500 nucleotides to about 2600 nucleotides, or from 2600 nucleotides to 2631 nucleotides, of the nucleotide sequence depicted in FIGS. 11A and 11B and set forth in SEQ ID NO:4. A suitable nucleic acid includes a nucleic acid comprising a nucleotide sequence encoding an enzymatically active LSD1 polypeptide. The nucleic acid is exogenous to the host cell into which it is introduced.

An exogenous nucleic acid comprising a nucleotide sequence encoding an LSD1 polypeptide can be a recombinant expression vector, where suitable vectors include, e.g., recombinant retroviruses, lentiviruses, and adenoviruses; retroviral expression vectors, lentiviral expression vectors, nucleic acid expression vectors, and plasmid expression vectors. In some cases, the one or more exogenous nucleic acids is/are integrated into the genome of a cell. In other cases, the one or more exogenous nucleic acids persists in an episomal state in a cell.

Suitable expression vectors include, but are not limited to, viral vectors (e.g. viral vectors based on vaccinia virus; poliovirus; adenovirus (see, e.g., Li et al., Invest Opthalmol V is Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol V is Sci 38:2857 2863, 1997; Jomary et al., Gene Ther 4:683 690, 1997, Rolling et al., Hum Gene Ther 10:641 648, 1999; Ali et al., Hum Mol Genet. 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Examples of suitable viral vectors include, but are not limited, viral vectors based on retroviruses (including lentiviruses); adenoviruses; and adeno-associated viruses. An example of a suitable retrovirus-based vector is a vector based on murine moloney leukemia virus (MMLV); however, other recombinant retroviruses may also be used, e.g., Avian Leukosis Virus, Bovine Leukemia Virus, Murine Leukemia Virus (MLV), Mink-Cell focus-Inducing Virus, Murine Sarcoma Virus, Reticuloendotheliosis virus, Gibbon Abe Leukemia Virus, Mason Pfizer Monkey Virus, or Rous Sarcoma Virus, see, e.g., U.S. Pat. No. 6,333,195.

In other cases, the retrovirus-based vector is a lentivirus-based vector, (e.g., Human

Immunodeficiency Virus-1 (HIV-1); Simian Immunodeficiency Virus (SIV); or Feline Immunodeficiency Virus (FIV)), See, e.g., Johnston et al., (1999), Journal of Virology, 73(6):4991-5000 (FIV); Negre D et al., (2002), Current Topics in Microbiology and Immunology, 261:53-74 (SIV); Naldini et al., (1996), Science, 272:263-267 (HIV).

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

Treatment Methods; Administering an Agent that Increases LSD1 Enzymatic Activity and/or Levels

The present disclosure provides methods of treating an immunodeficiency virus infection in an individual, the methods generally involving co-administering to the individual an agent that reactivates latent HIV and an anti-HIV agent. The present disclosure provides methods for reducing the reservoir of latent immunodeficiency virus in an individual by administering to the individual an effective amount of an agent that increases LSD1 enzymatic activity and/or LSD1 levels. Suitable agents are described above.

An effective amount of an agent that increases LSD1 enzymatic activity and/or LSD1 levels is an amount that reactivates latent HIV and reduces the reservoir of latent HIV in an individual by at least about 20%, at least about 30%, at least about 40%, at least about 50%, at least about 60%, at least about 70%, at least about 80%, or at least about 90%. A “reduction in the reservoir of latent HIV” (also referred to as “reservoir of latently infected cells”) is a reduction in the number of cells in the individual that harbor a latent HIV infection. Whether the reservoir of latently infected cells is reduced can be determined using any known method, including the method described in Blankson et al. (2000) J. Infect. Disease 182(6):1636-1642.

In some embodiments, a subject method of treating an immunodeficiency virus infection in an individual in need thereof involves: a) administering to the individual an agent that increases LSD1 enzymatic activity and/or LSD1 levels; and b) administering to the individual an effective amount of an agent that inhibits an immunodeficiency virus function. The immunodeficiency virus function can be selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity. Administering to the individual an agent that increases LSD1 enzymatic activity and/or LSD1 levels results in reactivation of latent immunodeficiency virus. Administering an agent that inhibits an immunodeficiency virus function can result in one or both of: a reduction of immunodeficiency virus load in the individual; and an increase in the number of CD4⁺ T cells in the individual.

In some embodiments, an agent that increases LSD1 enzymatic activity and/or levels is administered in combination therapy with: 1) one or more nucleoside reverse transcriptase inhibitors (e.g., Combivir, Epivir, Hivid, Retrovir, Videx, Zerit, Ziagen, etc.); 2) one or more non-nucleoside reverse transcriptase inhibitors (e.g., Rescriptor, Sustiva, Viramune, etc.); 3) one or more protease inhibitors (e.g., Agenerase, Crixivan, Fortovase, Invirase, Kaletra, Norvir, Viracept, etc.); 4) anti-HIV agent such as a protease inhibitor and a nucleoside reverse transcriptase inhibitor; 5) anti-HIV agent such as a protease inhibitor, a nucleoside reverse transcriptase inhibitor, and a non-nucleoside reverse transcriptase inhibitor; 6) anti-HIV agent such as a protease inhibitor and a non-nucleoside reverse transcriptase inhibitor. Other combinations of an effective amount of an agent that increases LSD1 enzymatic activity and/or levels with one or more anti-HIV agents, such as one or more of a protease inhibitor, a nucleoside reverse transcriptase inhibitor, and a non-nucleoside reverse transcriptase inhibitor, are contemplated.

Any of a variety of methods can be used to determine whether a treatment method is effective. For example, methods of determining whether the methods of the invention are effective in reducing immunodeficiency virus (e.g., HIV) viral load, and/or treating an immunodeficiency virus (e.g., HIV) infection, are any known test for indicia of immunodeficiency virus (e.g., HIV) infection, including, but not limited to, measuring viral load, e.g., by measuring the amount of immunodeficiency virus (e.g., HIV) in a biological sample, e.g., using a polymerase chain reaction (PCR) with primers specific for an immunodeficiency virus (e.g., HIV) polynucleotide sequence; detecting and/or measuring a polypeptide encoded by an immunodeficiency virus (e.g., HIV), e.g., p24, gp120, reverse transcriptase, using, e.g., an immunological assay such as an enzyme-linked immunosorbent assay (ELISA) with an antibody specific for the polypeptide; and measuring the CD4⁺ T cell count in the individual.

### Formulations, Dosages, and Routes of Administration

In general, active agents (e.g., an agent that inhibits LSD1 activity; an agent that reduces the level of LSD1; an agent that inhibits LSD1-mediated demethylation of methylated Tat; an agent that inhibits an activity of a CoREST polypeptide independently of any directe effect on LSD1; an agent that increases the level and/or activity of LSD1) are prepared in a pharmaceutically acceptable composition(s) for delivery to a host. In the context of reducing immunodeficiency virus transcription, the terms “active agent,” “drug,” “agent,” “therapeutic agent,” and the like are used interchangeably herein to refer to an agent that inhibits enzymatic activity of LSD1 and/or reduces the level of LSD1 and/or that inhibits LSD1-mediated demethylation of methylated Tat. In the context of reactivating latent HIV, the terms “active agent,” “drug,” “agent,” “therapeutic agent,” and the like are used interchangeably herein to refer to an agent that increases the level and/or enzymatic activity of LSD1.

Pharmaceutically acceptable carriers preferred for use with active agents (and optionally one or more additional therapeutic agent) may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, and microparticles, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. A composition comprising an active agent (and optionally one or more additional therapeutic agent) may also be lyophilized using means well known in the art, for subsequent reconstitution and use according to the invention.

### Formulations

An active agent is administered to an individual in need thereof in a formulation with a pharmaceutically acceptable excipient(s). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc. For the purposes of the following description of formulations, “active agent” includes an active agent as described above, and optionally one or more additional therapeutic agent.

In a subject method, an active agent may be administered to the host using any convenient means capable of resulting in the desired degree of: 1) reduction of immunodeficiency virus transcription; or 2) reactivation of latent immunodeficiency virus. Thus, an active agent can be incorporated into a variety of formulations for therapeutic administration. For example, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. In an exemplary embodiment, an active agent is formulated as a gel, as a solution, or in some other form suitable for intravaginal administration. In a further exemplary embodiment, an active agent is formulated as a gel, as a solution, or in some other form suitable for rectal (e.g., intrarectal) administration.

In pharmaceutical dosage forms, an active agent may be administered in the form of its pharmaceutically acceptable salts, or it may also be used alone or in appropriate association, as well as in combination, with other pharmaceutically active compounds. The following methods and excipients are merely exemplary and are in no way limiting.

In some embodiments, an active is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. An active agent can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise the active agent(s) in a composition as a solution in sterile water, normal saline or another pharmaceutically acceptable carrier.

Unit dosage forms for intravaginal or intrarectal administration such as syrups, elixirs, gels, and suspensions may be provided wherein each dosage unit, for example, teaspoonful, tablespoonful, tablet, unit gel volume, or suppository, contains a predetermined amount of the composition containing one or more active agents.

The term “unit dosage form,” as used herein, refers to physically discrete units suitable as unitary dosages for human and animal subjects, each unit containing a predetermined quantity of an active agent, calculated in an amount sufficient to produce the desired effect in association with a pharmaceutically acceptable diluent, carrier or vehicle. The specifications for a given active agent will depend in part on the particular compound employed and the effect to be achieved, and the pharmacodynamics associated with each compound in the host.

Other modes of administration will also find use with the subject invention. For instance, an active agent can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), e.g. about 1% to about 2%.

An active agent can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

An active agent will in some embodiments be formulated for vaginal delivery. A subject formulation for intravaginal administration comprises an active agent formulated as an intravaginal bioadhesive tablet, intravaginal bioadhesive microparticle, intravaginal cream, intravaginal lotion, intravaginal foam, intravaginal ointment, intravaginal paste, intravaginal solution, or intravaginal gel.

An active agent will in some embodiments be formulated for rectal delivery. A subject formulation for intrarectal administration comprises an active agent formulated as an intrarectal bioadhesive tablet, intrarectal bioadhesive microparticle, intrarectal cream, intrarectal lotion, intrarectal foam, intrarectal ointment, intrarectal paste, intrarectal solution, or intrarectal gel.

A subject formulation comprising an active agent includes one or more of an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Tablets comprising an active agent may be coated with a suitable film-forming agent, e.g., hydroxypropylmethyl cellulose, hydroxypropyl cellulose or ethyl cellulose, to which a suitable excipient may optionally be added, e.g., a softener such as glycerol, propylene glycol, diethylphthalate, or glycerol triacetate; a filler such as sucrose, sorbitol, xylitol, glucose, or lactose; a colorant such as titanium hydroxide; and the like.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Dosages

Although the dosage used will vary depending on the clinical goals to be achieved, a suitable dosage range of an active agent is one which provides up to about 1 mg to about 1000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, or from about 500 mg to about 1000 mg of an active agent can be administered in a single dose.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

In some embodiments, a single dose of an active agent is administered. In other embodiments, multiple doses of an active agent are administered. Where multiple doses are administered over a period of time, an active agent is administered twice daily (qid), daily (qd), every other day (qod), every third day, three times per week (tiw), or twice per week (biw) over a period of time. For example, an active agent is administered qid, qd, qod, tiw, or biw over a period of from one day to about 2 years or more. For example, an active agent is administered at any of the aforementioned frequencies for one week, two weeks, one month, two months, six months, one year, or two years, or more, depending on various factors.

Where two different active agents are administered, a first active agent and a second active agent can be administered in separate formulations. A first active agent and a second active agent can be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

### Routes of Administration

An active agent is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, transdermal, subcutaneous, intradermal, topical application, intravenous, vaginal, nasal, and other parenteral routes of administration. In some embodiments, an active agent is administered via an intravaginal route of administration. In other embodiments, an active agent is administered via an intrarectal route of administration. Routes of administration may be combined, if desired, or adjusted depending upon the agent and/or the desired effect. The composition can be administered in a single dose or in multiple doses.

An active agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, vaginal, transdermal, subcutaneous, intramuscular, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

An active agent can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as the number of viral particles per unit blood. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts (wherein the term “host” is used interchangeably herein with the terms “subject” and “patient”) are treatable according to the subject methods. Generally such hosts are “mammals” or “mammalian,” where these terms are used broadly to describe organisms which are within the class mammalia, and primates (e.g., humans, chimpanzees, and monkeys), that are susceptible to immunodeficiency virus (e.g., HIV) infection. In many embodiments, the hosts will be humans.

### Kits, Containers, Devices, Delivery Systems

Kits with unit doses of the active agent, e.g. in oral, vaginal, rectal, transdermal, or injectable doses (e.g., for intramuscular, intravenous, or subcutaneous injection), are provided. In such kits, in addition to the containers containing the unit doses will be an informational package insert describing the use and attendant benefits of the drugs in treating an immunodeficiency virus (e.g., an HIV) infection. Suitable active agents and unit doses are those described herein above.

In many embodiments, a subject kit will further include instructions for practicing the subject methods or means for obtaining the same (e.g., a website URL directing the user to a webpage which provides the instructions), where these instructions are typically printed on a substrate, which substrate may be one or more of: a package insert, the packaging, formulation containers, and the like.

In some embodiments, a subject kit includes one or more components or features that increase patient compliance, e.g., a component or system to aid the patient in remembering to take the active agent at the appropriate time or interval. Such components include, but are not limited to, a calendaring system to aid the patient in remembering to take the active agent at the appropriate time or interval.

The present invention provides a delivery system comprising an active agent that inhibits LSD1 enzymatic activity. In some embodiments, the delivery system is a delivery system that provides for injection of a formulation comprising an active agent subcutaneously, intravenously, or intramuscularly. In other embodiments, the delivery system is a vaginal or rectal delivery system.

In some embodiments, an active agent is packaged for oral administration. The present invention provides a packaging unit comprising daily dosage units of an active agent. For example, the packaging unit is in some embodiments a conventional blister pack or any other form that includes tablets, pills, and the like. The blister pack will contain the appropriate number of unit dosage forms, in a sealed blister pack with a cardboard, paperboard, foil, or plastic backing, and enclosed in a suitable cover. Each blister container may be numbered or otherwise labeled, e.g., starting with day 1.

In some embodiments, a subject delivery system comprises an injection device. Exemplary, non-limiting drug delivery devices include injections devices, such as pen injectors, and needle/syringe devices. In some embodiments, the invention provides an injection delivery device that is pre-loaded with a formulation comprising an effective amount of an active agent that inhibits LSD1 enzymatic activity. For example, a subject delivery device comprises an injection device pre-loaded with a single dose of an active agent that inhibits LSD1 enzymatic activity. A subject injection device can be re-usable or disposable.

Pen injectors are well known in the art. Exemplary devices which can be adapted for use in the present methods are any of a variety of pen injectors from Becton Dickinson, e.g., BD™ Pen, BD™ Pen II, BD™ Auto-Injector; a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable.

The present invention provides a delivery system for vaginal or rectal delivery of an active agent to the vagina or rectum of an individual. The delivery system comprises a device for insertion into the vagina or rectum. In some embodiments, the delivery system comprises an applicator for delivery of a formulation into the vagina or rectum; and a container that contains a formulation comprising an active agent. In these embodiments, the container (e.g., a tube) is adapted for delivering a formulation into the applicator. In other embodiments, the delivery system comprises a device that is inserted into the vagina or rectum, which device includes an active agent. For example, the device is coated with, impregnated with, or otherwise contains a formulation comprising the active agent.

In some embodiments, the vaginal or rectal delivery system is a tampon or tampon-like device that comprises a subject formulation. Drug delivery tampons are known in the art, and any such tampon can be used in conjunction with a subject drug delivery system. Drug delivery tampons are described in, e.g., U.S. Pat. No. 6,086,909 If a tampon or tampon-like device is used, there are numerous methods by which an active agent can be incorporated into the device. For example, the drug can be incorporated into a gel-like bioadhesive reservoir in the tip of the device. Alternatively, the drug can be in the form of a powdered material positioned at the tip of the tampon. The drug can also be absorbed into fibers at the tip of the tampon, for example, by dissolving the drug in a pharmaceutically acceptable carrier and absorbing the drug solution into the tampon fibers. The drug can also be dissolved in a coating material which is applied to the tip of the tampon. Alternatively, the drug can be incorporated into an insertable suppository which is placed in association with the tip of the tampon.

In other embodiments, the drug delivery device is a vaginal or rectal ring. Vaginal or rectal rings usually consist of an inert elastomer ring coated by another layer of elastomer containing an active agent to be delivered. The rings can be easily inserted, left in place for the desired period of time (e.g., up to 7 days), then removed by the user. The ring can optionally include a third, outer, rate-controlling elastomer layer which contains no drug. Optionally, the third ring can contain a second drug for a dual release ring. The drug can be incorporated into polyethylene glycol throughout the silicone elastomer ring to act as a reservoir for drug to be delivered.

In other embodiments, a subject vaginal or rectal delivery system is a vaginal or rectal sponge. The active agent is incorporated into a silicone matrix which is coated onto a cylindrical drug-free polyurethane sponge, as described in the literature.

Pessaries, tablets, and suppositories are other examples of drug delivery systems which can be used in the present invention. These systems have been described extensively in the literature.

Bioadhesive microparticles constitute still another drug delivery system suitable for use in the present invention. This system is a multi-phase liquid or semi-solid preparation which does not seep from the vagina or rectum as do many suppository formulations. The substances cling to the wall of the vagina or rectum and release the drug over a period of time. Many of these systems were designed for nasal use but can be used in the vagina or rectum as well (e.g. U.S. Pat. No. 4,756,907). The system may comprise microspheres with an active agent; and a surfactant for enhancing uptake of the drug. The microparticles have a diameter of 10-100 μm and can be prepared from starch, gelatin, albumin, collagen, or dextran.

Another system is a container comprising a subject formulation (e.g., a tube) that is adapted for use with an applicator. The active agent is incorporated into creams, lotions, foams, paste, ointments, and gels which can be applied to the vagina or rectum using an applicator. Processes for preparing pharmaceuticals in cream, lotion, foam, paste, ointment and gel formats can be found throughout the literature. An example of a suitable system is a standard fragrance free lotion formulation containing glycerol, ceramides, mineral oil, petrolatum, parabens, fragrance and water such as the product sold under the trademark JERGENS™ (Andrew Jergens Co., Cincinnati, Ohio). Suitable nontoxic pharmaceutically acceptable systems for use in the compositions of the present invention will be apparent to those skilled in the art of pharmaceutical formulations and examples are described in Remington's Pharmaceutical Sciences, 19th Edition, A. R. Gennaro, ed., 1995. The choice of suitable carriers will depend on the exact nature of the particular vaginal or rectal dosage form desired, e.g., whether the active ingredient(s) is/are to be formulated into a cream, lotion, foam, ointment, paste, solution, or gel, as well as on the identity of the active ingredient(s). Other suitable delivery devices are those described in U.S. Pat. No. 6,476,079.

### Combination Therapy

In some embodiments, two or more agents that inhibit LSD1 enzymatic activity, that reduce the level of LSD1 in a cell, or that reduce LSD1-mediated demethylation of Tat, are administered in combination therapy. For example, the following agents can be co-administered: 1) a small molecule inhibitor of LSD1 enzymatic activity and a dominant negative CoREST polypeptide; 2) a small molecule inhibitor of LSD1 enzymatic activity and a BHC80 polypeptide; 3) a small molecule inhibitor of LSD1 enzymatic activity and an HDAC inhibitor; 4) a small molecule inhibitor of LSD1 enzymatic activity and an interfering nucleic acid that reduces CoREST expression. Other combinations are also encompassed by the present disclosure.

In some embodiments, an active agent is administered in combination therapy with one or more additional therapeutic agents. Suitable additional therapeutic agents include agents that inhibit one or more functions of an immunodeficiency virus; agents that treat or ameliorate a symptom of an immunodeficiency virus infection; agents that treat an infection that occurs secondary to an immunodeficiency virus infection; and the like.

Therapeutic agents include, e.g., beta-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™), trisodium phosphonoformate (Foscarnet™), famcyclovir, pencyclovir, valacyclovir, nucleic acid/replication inhibitors, interferon, zidovudine (AZT, Retrovir™), didanosine (dideoxyinosine, ddI, Videx™), stavudine (d4T, Zerit™), zalcitabine (dideoxycytosine, ddC, Hivid™), nevirapine (Viramune™), lamivudine (Epivir™, 3TC), protease inhibitors, saquinavir (Invirase™, Fortovase™), ritonavir (Norvir™), nelfinavir (Viracept™), efavirenz (Sustiva™), abacavir (Ziagen™), amprenavir (Agenerase™) indinavir (Crixivan™), ganciclovir, AzDU, delavirdine (Rescriptor™), kaletra, trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparaginase and combinations thereof. Anti-HIV agents are those in the preceding list that specifically target a function of one or more HIV proteins.

In some embodiments, a subject antibody is administered in combination therapy with two or more anti-HIV agents. For example, a subject antibody can be administered in combination therapy with one, two, or three nucleoside reverse transcriptase inhibitors (e.g., Combivir, Epivir, Hivid, Retrovir, Videx, Zerit, Ziagen, etc.). A subject antibody can be administered in combination therapy with one or two non-nucleoside reverse transcriptase inhibitors (e.g., Rescriptor, Sustiva, Viramune, etc.). A subject antibody can be administered in combination therapy with one or two protease inhibitors (e.g., Agenerase, Crixivan, Fortovase, Invirase, Kaletra, Norvir, Viracept, etc.). A subject antibody can be administered in combination therapy with a protease inhibitor and a nucleoside reverse transcriptase inhibitor. A subject antibody can be administered in combination therapy with a protease inhibitor, a nucleoside reverse transcriptase inhibitor, and a non-nucleoside reverse transcriptase inhibitor. A subject antibody can be administered in combination therapy with a protease inhibitor and a non-nucleoside reverse transcriptase inhibitor. Other combinations of a subject antibody with one or more of a protease inhibitor, a nucleoside reverse transcriptase inhibitor, and a non-nucleoside reverse transcriptase inhibitor are contemplated.

In some embodiments, a subject treatment method involves administering: a) an active agent (e.g.: 1) an agent that inhibits LSD1 enzymatic activity; 2) an agent that reduces the level of LSD1; 3) an agent that inhibits LSD1-mediated demethylation of methylated Tat; 4) an agent that increases LSD1 activity and/or levels); and b) an agent that inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity.

In some embodiments, a subject treatment method involves administering: a) an active agent (e.g.: 1) an agent that inhibits LSD1 enzymatic activity; 2) an agent that reduces the level of LSD1; 3) an agent that inhibits LSD1-mediated demethylation of methylated Tat; 4) an agent that increases LSD1 activity and/or levels); and b) an HIV inhibitor, where suitable HIV inhibitors include, but are not limited to, one or more nucleoside/nucleotide reverse transcriptase inhibitors (NRTIs), non-nucleoside reverse transcriptase inhibitors (NNRTIs), protease inhibitors (PIs), fusion inhibitors, integrase inhibitors, chemokine receptor (e.g., CXCR4, CCR5) inhibitors, and hydroxyurea.

Nucleoside reverse transcriptase inhibitors include, but are not limited to, abacavir (ABC; ZIAGEN™), didanosine (dideoxyinosine (ddI); VIDEX™), lamivudine (3TC; EPIVIR™), stavudine (d4T; ZERIT™, ZERIT XR™), zalcitabine (dideoxycytidine (ddC); HIVID™), zidovudine (ZDV, formerly known as azidothymidine (AZT); RETROVIR™), abacavir, zidovudine, and lamivudine (TRIZIVIR™), zidovudine and lamivudine (COMBIVIR™), and emtricitabine (EMTRIVA™). Nucleotide reverse transcriptase inhibitors include tenofovir disoproxil fumarate (VIREAD™). Non-nucleoside reverse transcriptase inhibitors for HIV include, but are not limited to, nevirapine (VIRAMUNE™), delavirdine mesylate (RESCRTPTOR™), and efavirenz (SUSTIVA™).

Protease inhibitors (PIs) for treating HIV infection include amprenavir (AGENERASE™), saquinavir mesylate (FORTOVASE™, INVIRASE™.), ritonavir (NORVIR™), indinavir sulfate (CRIXIVAN™), nelfmavir mesylate (VIRACEPT™), lopinavir and ritonavir (KALETRA™), atazanavir (REYATAZ™), and fosamprenavir (LEXIVA™).

Fusion inhibitors prevent fusion between the virus and the cell from occurring, and therefore, prevent HIV infection and multiplication. Fusion inhibitors include, but are not limited to, enfuvirtide (FUZEON™), Lalezari et al., New England J. Med., 348:2175-2185 (2003); and maraviroc (SELZENTRY™, Pfizer).

An integrase inhibitor blocks the action of integrase, preventing HIV-1 genetic material from integrating into the host DNA, and thereby stopping viral replication. Integrase inhibitors include, but are not limited to, raltegravir (ISENTRESS™, Merck); and elvitegravir (GS 9137, Gilead Sciences).

Maturation inhibitors include, e.g., bevirimat (3β-(3-carboxy-3-methyl-butanoyloxy) lup-20(29)-en-28-oic acid); and Vivecon (MPC9055).

In some embodiments, a subject treatment method involves administering: a) an active agent (e.g.: 1) an agent that inhibits LSD1 enzymatic activity; 2) an agent that reduces the level of LSD1; 3) an agent that inhibits LSD1-mediated demethylation of methylated Tat; 4) an agent that increases LSD1 activity and/or levels); and b) one or more of: (1) an HIV protease inhibitor selected from amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, RO0334649, KNI-272, DPC-681, DPC-684, GW640385X, DG17, PPL-100, DG35, and AG 1859; (2) an HIV non-nucleoside inhibitor of reverse transcriptase selected from capravirine, emivirine, delaviridine, efavirenz, nevirapine, (+) calanolide A, etravirine, GW5634, DPC-083, DPC-961, DPC-963, MN-150, and TMC-120, TMC-278 (rilpivirene), efavirenz, BILR 355 BS, VRX 840773, UK-453061, and RDEA806; (3) an HIV nucleoside inhibitor of reverse transcriptase selected from zidovudine, emtricitabine, didanosine, stavudine, zalcitabine, lamivudine, abacavir, amdoxovir, elvucitabine, alovudine, MIV-210, racivir, D-d4FC, emtricitabine, phosphazide, fozivudine tidoxil, apricitibine (AVX754), amdoxovir, KP-1461, and fosalvudine tidoxil (formerly HDP 99.0003); (4) an HIV nucleotide inhibitor of reverse transcriptase selected from tenofovir and adefovir; (5) an HIV integrase inhibitor selected from curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, MK-0518 (raltegravir), BMS-538158, GSK364735C, BMS-707035, MK-2048, and BA 011; (6) a gp41 inhibitor selected from enfuvirtide, sifuvirtide, FB006M, and TRI-1144; (7) a CXCR4 inhibitor, such as AMD-070; (8) an entry inhibitor, such as SP01A; (9) a gp120 inhibitor, such as BMS-488043 and/or BlockAide/CR; (10) a G6PD and NADH-oxidase inhibitor, such as immunitin; (11) a CCR5 inhibitors selected from the group consisting of aplaviroc, vicriviroc, maraviroc, PRO-140, INCB15050, PF-232798 (Pfizer), and CCR5 mAb004; (12) another drug for treating HIV selected from BAS-100, SPI-452, REP 9, SP-01A, TNX-355, DES6, ODN-93, ODN-112, VGV-1, PA-457 (bevirimat), Ampligen, HRG214, Cytolin, VGX-410, KD-247, AMZ 0026, CYT 99007A-221 HIV, DEBIO-025, BAY 50-4798, MDXO10 (ipilimumab), PBS119, ALG 889, and PA-1050040 (PA-040); (13) any combinations or mixtures of the above.

As further examples, in some embodiments, a subject treatment method involves administering: a) an active agent (e.g.: 1) an agent that inhibits LSD1 enzymatic activity; 2) an agent that reduces the level of LSD1; 3) an agent that inhibits LSD1-mediated demethylation of methylated Tat; 4) an agent that increases LSD1 activity and/or levels); and b) one or more of: i) amprenavir (Agenerase; (3S)-oxolan-3-yl N-[(2S,3R)-3-hydroxy-4-[N-(2-methylpropyl)(4-aminobenzene)sulfonamido]-1-phenylbutan-2-yl]carbamate) in an amount of 600 mg or 1200 mg twice daily; ii) tipranavir (Aptivus; N-{3-[(1R)-1-[(2R)-6-hydroxy-4-oxo-2-(2-phenylethyl)-2-propyl-3,4-dihydro-2H-pyran-5-yl]propyl]phenyl}-5-(trifluoromethyl)pyridine-2-sulfonamide) in an amount of 500 mg twice daily; iii) idinavir (Crixivan; (2S)-1-[(2S,4R)-4-benzyl-2-hydroxy-4-{[(1S,2R)-2-hydroxy-2,3-dihydro-1H-inden-1-yl]carbamoyl}butyl]-N-tert-butyl-4-(pyridin-3-ylmethyl)piperazine-2-carboxamide) in an amount of 800 mg three times daily; iv) saquinavir (Invirase; 2S)—N-[(2S,3R)-4-[(3S)-3-(tert-butylcarbamoyl)-decahydroisoquinolin-2-yl]-3-hydroxy-1-phenylbutan-2-yl]-2-(quinolin-2-ylformamido)butanediamide) in an amount of 1,000 mg twice daily; v) lopinavir and ritonavir (Kaleta; where lopinavir is 2S)—N-[(2S,4S,5S)-5-[2-(2,6-dimethylphenoxy)acetamido]-4-hydroxy-1,6-diphenylhexan-2-yl]-3-methyl-2-(2-oxo-1,3-diazinan-1-yl)butanamide; and ritonavir is 1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl})carbamoyl]amino}butanamido]-1,6-diphenylhexan-2-yl]carbamate) in an amount of 133 mg twice daily; yl) fosamprenavir (Lexiva; {[(2R,3S)-1-[N-(2-methylpropyl)(4-aminobenzene)sulfonamido]-3-({[(3S)-oxolan-3-yloxy]carbonyl}amino)-4-phenylbutan-2-yl]oxy}phosphonic acid) in an amount of 700 mg or 1400 mg twice daily); vii) ritonavir (Norvir) in an amount of 600 mg twice daily; viii) nelfinavir (Viracept; (3S,4aS,8aS)—N-tert-butyl-2-[(2R,3R)-2-hydroxy-3-[(3-hydroxy-2-methylphenyl)formamido]-4-(phenylsulfanyl)butyl]-decahydroisoquinoline-3-carboxamide) in an amount of 750 mg three times daily or in an amount of 1250 mg twice daily; ix) Fuzeon (Acetyl-YTSLIHSLIEESQNQ QEKNEQELLELDKWASLWNWF-amide; SEQ ID NO:57) in an amount of 90 mg twice daily; x) Combivir in an amount of 150 mg lamivudine (3TC; 2′,3′-dideoxy-3′-thiacytidine) and 300 mg zidovudine (AZT; azidothymidine) twice daily; xi) emtricitabine (Emtriva; 4-amino-5-fluoro-1-[2R,5S)-2-(hydroxymethyl)-1,3-oxathiolan-5-yl]-1,2-dihydropyrimidin-2-one) in an amount of 200 mg once daily; xii) Epzicom in an amount of 600 mg abacavir (ABV; {(1S,4R)-4-[2-amino-6-(cyclopropylamino)-9H-purin-9-yl]cyclopent-2-en-1-yl}methanol) and 300 mg 3TC once daily; xiii) zidovudine (Retrovir; AZT or azidothymidine) in an amount of 200 mg three times daily; xiv) Trizivir in an amount of 150 mg 3TC and 300 mg ABV and 300 mg AZT twice daily; xv) Truvada in an amount of 200 mg emtricitabine and 300 mg tenofovir (({[(2R)-1-(6-amino-9H-purin-9-yl)propan-2-yl]oxy}methyl)phosphonic acid) once daily; xvi) didanosine (Videx; 2′,3′-dideoxyinosine) in an amount of 400 mg once daily; xvii) tenofovir (Viread) in an amount of 300 mg once daily; xviii) abacavir (Ziagen) in an amount of 300 mg twice daily; xix) atazanavir (Reyataz; methyl N-[(1S)-1-{[(2S,3S)-3-hydroxy-4-[(2S)-2-[(methoxycarbonyl)amino]-3,3-dimethyl-N′-{[4-(pyridin-2-yl)phenyl]methyl}butanehydrazido]-1-phenylbutan-2-yl]carbamoyl}-2,2-dimethylpropyl]carbamate) in an amount of 300 mg once daily or 400 mg once daily; xx) lamivudine (Epivir) in an amount of 150 mg twice daily; xxi) stavudine (Zerit; 2′-3′-didehydro-2′-3′-dideoxythymidine) in an amount of 40 mg twice daily; xxii) delavirdine (Rescriptor; N-[2-({4-[3-(propan-2-ylamino)pyridin-2-yl]piperazin-1-yl}carbonyl)-1H-indol-5-yl]methanesulfonamide) in an amount of 400 mg three times daily; xxiii) efavirenz (Sustiva; (4S)-6-chloro-4-(2-cyclopropylethynyl)-4-(trifluoromethyl)-2,4-dihydro-1H-3,1-benzoxazin-2-one) in an amount of 600 mg once daily); xxiv) nevirapine (Viramune; 11-cyclopropyl-4-methyl-5,11-dihydro-6H-dipyrido[3,2-b:2′,3′-e][1,4]diazepin-6-one) in an amount of 200 mg twice daily); xxv) bevirimat; and xxvi) Vivecon.

### Subjects Suitable for Treatment

The methods of the present disclosure are suitable for treating individuals who have an immunodeficiency virus infection, e.g., who have been diagnosed as having an immunodeficiency virus infection.

The methods of the present disclosure are suitable for treating individuals who have an HIV infection (e.g., who have been diagnosed as having an HIV infection), and individuals who are at risk of contracting an HIV infection. Such individuals include, but are not limited to, individuals with healthy, intact immune systems, but who are at risk for becoming HIV infected (“at-risk” individuals). At-risk individuals include, but are not limited to, individuals who have a greater likelihood than the general population of becoming HIV infected. Individuals at risk for becoming HIV infected include, but are not limited to, individuals at risk for HIV infection due to sexual activity with HIV-infected individuals. Individuals suitable for treatment include individuals infected with, or at risk of becoming infected with, HIV-1 and/or HIV-2 and/or HIV-3, or any variant thereof.

In some embodiments, subjects who have been diagnosed as having cancer are specifically excluded. In some embodiments, subjects who have been diagnosed as having a neurological disorder are specifically excluded.

### Screening Methods

The present disclosure provides screening methods, e.g., methods of identifying agents that modulate LSD1 enzymatic activity and/or LSD1 levels.

The present disclosure provides methods of identifying an agent that inhibits LSD1-mediated demethylation of a methylated Tat polypeptide. The methods generally comprise: a) contacting an LSD1 polypeptide and a methylated Tat polypeptide with a test agent; and b) determining the effect, if any, of the test agent on the methylation of the Tat polypeptide. A test agent that inhibits demethylation of the methylated Tat polypeptide by the LSD1 polypeptide, compared to the level of methylation of the Tat polypeptide in the absence of the test agent, is considered an inhibitor of LSD1-mediated methylation of HIV Tat.

An agent that inhibits demethylation of a methylated Tat polypeptide by an LSD1 polypeptide is considered a candidate agent for inhibiting HIV transcription, and is thus considered a candidate agent for treating an HIV infection in an individual.

The present disclosure provides methods of identifying an agent that increases LSD1-mediated demethylation of a methylated Tat polypeptide. The methods generally comprise: a) contacting an LSD1 polypeptide and a methylated Tat polypeptide with a test agent; and b) determining the effect, if any, of the test agent on the methylation of the Tat polypeptide. A test agent that increases LSD1-mediated demethylation of a methylated Tat polypeptide, compared to the level of methylation of the Tat polypeptide in the absence of the test agent, is considered an activator of LSD1-mediated methylation of HIV Tat.

An agent that increases demethylation of a methylated Tat polypeptide by an LSD1 polypeptide is considered a candidate agent for reactivating latent HIV integrated into the genome of an HIV-infected cell. Such an agent could be used in combination therapy with one or more anti-HIV agents.

By “test agent,” “candidate agent,” and grammatical equivalents thereof, which terms are used interchangeably herein, is meant any molecule (e.g. proteins (which herein includes proteins, polypeptides, and peptides); small (i.e., 5 Da-1000 Da, 100 Da-750 Da, 200 Da-500 Da, or less than 500 Da in size) organic or inorganic molecules; polysaccharides, polynucleotides, etc.) which are to be tested for activity in inhibiting methylation of a Tat polypeptide by an LSD1 polypeptide.

A variety of different test agents may be screened using a subject method. Candidate agents encompass numerous chemical classes, e.g., small organic compounds having a molecular weight of more than 50 daltons and less than about 10,000 daltons, less than about 5,000 daltons, or less than about 2,500 daltons. Test agents can comprise functional groups necessary for structural interaction with proteins, e.g., hydrogen bonding, and can include at least an amine, carbonyl, hydroxyl or carboxyl group, or at least two of the functional chemical groups. The test agents can comprise cyclical carbon or heterocyclic structures and/or aromatic or polyaromatic structures substituted with one or more of the above functional groups. Test agents are also found among biomolecules including peptides, saccharides, fatty acids, steroids, purines, pyrimidines, derivatives, structural analogs or combinations thereof.

Test agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In one embodiment, test agents are synthetic compounds. A number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the test agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In one embodiment, the test agents are organic moieties. In this embodiment, as is generally described in WO 94/24314, test agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

As used herein, the term “determining” refers to both quantitative and qualitative determinations and as such, the term “determining” is used interchangeably herein with “assaying,” “measuring,” and the like.

Suitable LSD1 polypeptides are described above. For example, an LSD1 polypeptide can comprise an amino acid sequence having at least about 75%, at least about 80%, at least about 85%, at least about 90%, at least about 95%, at least about 98%, at least about 99%, or 100%, amino acid sequence identity to a contiguous stretch of from about 750 amino acids to about 800 amino acids, or from about 800 amino acids to 852 amino acids, of the amino acid sequence depicted in FIG. 8 and set forth in SEQ ID NO:1. As another example, an LSD1 polypeptide can comprise an amino acid sequence having at least about 75%, at least about 80%, at least about 85%, at least about 90%, at least about 95%, at least about 98%, at least about 99%, or 100%, amino acid sequence identity to a contiguous stretch of from about 750 amino acids to about 800 amino acids, from about 800 amino acids to about 850 amino acids, or from about 850 amino acids to 876 amino acids, of the amino acid sequence depicted in FIG. 10 and set forth in SEQ ID NO:3.

As another example, an LSD1 polypeptide is encoded by a nucleotide sequence having at least about 75%, at least about 80%, at least about 85%, at least about 90%, at least about 95%, at least about 98%, at least about 99%, or 100%, nucleotide sequence identity to a contiguous stretch of from about 2400 nucleotides to about 2500 nucleotides, or from 2500 nucleotides to 2559 nucleotides, of the nucleotide sequence depicted in FIGS. 9A and 9B and set forth in SEQ ID NO:2. As another example, an LSD1 polypeptide is encoded by a nucleotide sequence having at least about 75%, at least about 80%, at least about 85%, at least about 90%, at least about 95%, at least about 98%, at least about 99%, or 100%, nucleotide sequence identity to a contiguous stretch of from about 2500 nucleotides to about 2600 nucleotides, or from 2600 nucleotides to 2631 nucleotides, of the nucleotide sequence depicted in FIGS. 11A and 11B and set forth in SEQ ID NO:4.

A methylated Tat polypeptide can have a length of from about 50 amino acids to about 105 amino acids, e.g., from about 50 aa to about 55 aa, from about 55 aa to about 60 aa, from about 60 aa to about 65 aa, from about 65 aa to about 70 aa, from about 70 aa to about 75 aa, from about 75 aa to about 80 aa, from about 80 aa to about 85 aa, from about 85 aa to about 90 aa, from about 90 aa to about 95 aa, from about 95 aa to about 100 aa, or from about 100 aa to about 105 aa; where the isolated, methylated Tat polypeptide comprises a methylated Lys at a position corresponding to Lys-51 of SEQ ID NO:5; and where the isolated, methylated Tat polypeptide comprises an amino acid sequence having at least about 75%, at least about 80%, at least about 85%, at least about 90%, at least about 95%, at least about 98%, at least about 99%, or 100% amino acid sequence identity to the amino acid sequence set forth in SEQ ID NO:5 (FIG. 12). A Tat lysine residue at a position corresponding to Lys-51 of the consensus Tat sequence can be monomethylated or dimethylated. Demethylation includes removal of the methyl group of monomethylated Tat; and removal of one or both methyl groups of dimethylated Tat.

The amino acid sequences of HIV Tat polypeptides are known, and any of these —sequences can be included in a subject acetylated Tat polypeptide. Numerous HIV Tat protein amino acid sequences are found under GenBank. Exemplary, non-limiting, HIV Tat protein amino acid sequences are found under GenBank Accession Nos. AAO26250, AAO26252, AAO26254, AAO26258, AAO26260, AAO26262, AAO26264, AAO26266, AAO26268, AAO26270, AAO26272, AAO26274, AAO26276, AAO26278, AAO26280, AAO26282, AAO26284, AAO26286, AAO26288, AAO26290, AAO26292, AAO26294, AAO26296, AAO26298, AAO26300, AAO26302, AAO26304, AAO26306, AAO26308; AAB50256; AAL12204; AAL12195; AAL12186; AAL12177; AAN47131; AAN47122; AAN47113; AAN47104; AAN03332; AAN03323; AAN03314; AAN03305; AAN03296; AAN03287; AAN03278; AAN31592; AAN64126; AAN64117; AAN64108; AAN64099; AAN64090; AAN64080; K02013; AAL29460; and as shown in FIGS. 13A and 13B (SEQ ID NOs:12-34; and consensus Tat sequence SEQ ID NO:5). Additional HIV Tat amino acid sequences are found in Peloponese et al. (1999) J. Biol. Chem. 274:11473-11478; and Goldstein (1996) Nat. Med. 2:960-964.

A methylated substrate for an LSD1 polypeptide can comprise a monomethylated and/or a dimethylated lysine. In some embodiments, a methylated LSD1 substrate is a methylated HIV Tat polypeptide. In some embodiments, a methylated Tat polypeptide comprises a methylated lysine at a position corresponding to Lys-51 of the amino acid sequence depicted in FIG. 12 and set forth in SEQ ID NO:5, where the methylated lysine is monomethylated or dimethylated. In some embodiments, a methylated Tat polypeptide comprises the amino acid sequence SYGRKK^{Me}RRQR (SEQ ID NO:6), or a variation thereof, where the methylated lysine is monomethylated or dimethylated. Suitable methylated Tat polypeptides are described in, e.g., U.S. Patent Publication No. 2009/0233267.

In some embodiments, a methylated Tat polypeptide includes heterologous amino acid sequences, e.g., a methylated Tat polypeptide may be a fusion protein that comprises a methylated Tat polypeptide and a fusion partner, where the fusion partner is a heterologous polypeptide (e.g., a polypeptide other than Tat). Heterologous polypeptides are polypeptides other than Tat, and include, but are not limited to, polypeptide carriers (discussed in more detail below); immunological tags such as epitope tags, including, but not limited to, hemagglutinin (e.g., CYPYDVPDYA; SEQ ID NO:58), FLAG (e.g., DYKDDDDK; SEQ ID NO:59), c-myc (EQKLISEEDL; SEQ ID NO:60) and the like; proteins that provide for a detectable signal, including, but not limited to, fluorescent proteins, enzymes (e.g., β-galactosidase, alkaline phosphatase, luciferase, horse radish peroxidase, etc.), and the like; polypeptides that facilitate purification or isolation of the fusion protein, e.g., metal ion binding polypeptides such as 6His tags, glutathione-5-transferase; polypeptides that facilitate transport across a eukaryotic cell membrane; and the like.

Suitable fluorescent proteins include, but are not limited to, a green fluorescent protein (GFP), including, but not limited to, a “humanized” version of a GFP, e.g., wherein codons of the naturally-occurring nucleotide sequence are changed to more closely match human codon bias; a GFP derived from Aequoria victoria or a derivative thereof, e.g., a “humanized” derivative such as Enhanced GFP, which are available commercially, e.g., from Clontech, Inc.; a GFP from another species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); any of a variety of fluorescent and colored proteins from Anthozoan species, as described in, e.g., Matz et al. (1999) Nature Biotechnol. 17:969-973; a red fluorescent protein; a yellow fluorescent protein; and the like. Where the fusion partner is an enzyme that yields a detectable product, the product can be detected using an appropriate means, e.g., β-galactosidase can, depending on the substrate, yield colored product, which is detected spectrophotometrically, or a fluorescent product; luciferase can yield a luminescent product detectable with a luminometer; etc.

In some embodiments, a methylated Tat polypeptide is detectably labeled. Various labels include radioisotopes, fluorescers (e.g., fluorescent dyes), chemiluminescers, enzymes, a member of a specific binding pair, particles, e.g. magnetic particles, and the like. Specific binding pairs include, but are not limited to, biotin and streptavidin; digoxin and antidigoxin; lectin and carbohydrate moieties; antibody and hapten; antibody and antigen; etc.

A methylated Tat polypeptide may be synthesized chemically or enzymatically, may be produced recombinantly, may be isolated from a natural source, or a combination of the foregoing. A methylated Tat polypeptide may be isolated from natural sources using standard methods of protein purification known in the art, including, but not limited to, high performance liquid chromatography, exclusion chromatography, gel electrophoresis, affinity chromatography, or other purification technique. One may employ solid phase peptide synthesis techniques, where such techniques are known to those of skill in the art. See Jones, The Chemical Synthesis of Peptides (Clarendon Press, Oxford)(1994). Generally, in such methods a peptide is produced through the sequential additional of activated monomeric units to a solid phase bound growing peptide chain. Peptides can be synthesized in solution or on a solid support in accordance with conventional techniques. Various automatic synthesizers are commercially available and can be used in accordance with known protocols. See, for example, Stewart and Young, Solid Phase Peptide Synthesis, 2d. ed., Pierce Chemical Co. (1984); Tam et al., J. Am. Chem. Soc. 105:6442 (1983); Merrifield, Science 232:341-347 (1986); and Barany and Merrifield, The Peptides, Gross and Meienhofer, eds., Academic Press, New York, pp. 1-284 (1979), each of which is incorporated herein by reference. Well-established recombinant DNA techniques can be employed for production of a methylated Tat polypeptide.

In some embodiments, the Tat polypeptide is methylated in a cell-free reaction in vitro, either after synthesis or during synthesis, or, e.g., after isolation from a naturally-occurring source of a Tat polypeptide. For example, a Tat polypeptide is contacted with a SET domain-containing polypeptide (e.g., Set9) in a buffer containing 50 mM Tris-HCl (pH 8.5), 5 mM MgCl₂, 4 mM dithiothreitol (DTT) and S-adenosyl methionine (SAM). In some embodiments, a Lys⁵¹ methylated Tat polypeptide is detectably labeled during synthesis of the Tat polypeptide, e.g., using a radioactively labeled methyl donor.

Suitable SET domain-containing polypeptides include, e.g., a Set9 polypeptide, a SETDB1 polypeptide, and a SETDB2 polypeptide. For Set9 amino acid sequences, see U.S. Patent Publication No. 2009/0233267 (e.g., FIG. 12A and SEQ ID NO:31 of U.S. Patent Publication No. 2009/0233267); GenBank Accession No. NP_{—}085151; and GenBank Accession Nos. Q8WTS6, AAL69901, and AAI2105. For SETDB1 polypeptides and SETDB2 polypeptides, see, e.g., vanDuyne et al. (2008) Retrovirol. 5:40; GenBank Accession No. AAH09362 for an amino acid sequence of human SETDB1; and GenBank Accession Nos. AAH47434 and AAH17078 for amino acid sequences of human SETDB2.

Amino acid sequences of Set9 polypeptides are known in the art. For example, a human Set9 amino acid sequence is found in GenBank Accession No. NP_{—}085151. Other Set9 amino acid sequences are provided in GenBank Accession Nos. Q8WTS6, AAL69901, and AAI21056. A suitable Set9 polypeptide includes a polypeptide comprising an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to a contiguous stretch of from about 250 amino acids to about 300 amino acids, or from about 300 amino acids to about 365 amino acids, of the amino acid sequence provided in GenBank Accession No. NP_{—}085151; where the Set9 polypeptide is enzymatically active, e.g., is capable of methylating a lysine at a position corresponding to Lys-51 of the Tat polypeptide set forth in FIG. 12.

A Tat polypeptide can also be methylated post-translationally in a living cell (e.g., a eukaryotic cell such as a mammalian cell line) in vitro, e.g., Lys-51 is methylated post-translationally following synthesis of the Tat polypeptide. For example, the cell can include a Set9 polypeptide, e.g., the cell can include an endogenous Set9 polypeptide, or can be genetically modified with a nucleic acid that comprises a nucleotide sequence encoding a Set9 polypeptide. Alternatively, the cell could include a SETDB1 polypeptide or a SETDB2 polypeptide, e.g., the cell can include an endogenous SETDB1 polypeptide or an endogenous SETDB2 polypeptide, or can be genetically modified with a nucleic acid that comprises a nucleotide sequence encoding a SETDB1 polypeptide or a SETDB2 polypeptide. In some embodiments, a Lys⁵¹ methylated Tat polypeptide is detectably labeled during synthesis of the Tat polypeptide, e.g., using a radioactively labeled methyl donor.

Methylated Tat polypeptide synthesized by a living eukaryotic cell can be recovered using standard methods for protein purification. In some embodiments, the Tat polypeptide that is methylated by a living eukaryotic cell is a fusion protein comprising a moiety that facilitates purification (e.g., a binding moiety), e.g., glutathione-5-transferase (GST), 6His, etc., and the methylated Tat polypeptide is purified using a separation medium appropriate to the binding moiety.

In some embodiments, a methylated Tat polypeptide is a fusion protein, e.g., a polypeptide comprising a methylated Tat polypeptide and a heterologous (non-Tat) polypeptide (e.g., a fusion partner), where suitable heterologous polypeptides (fusion partners) include, e.g., an epitope tag; enzymes that act on a substrate to yield a detectable product (e.g., alkaline phosphatase, luciferase, horse radish peroxidase, β-galactosidase, etc.); fluorescent proteins (e.g., a green fluorescent protein, a yellow fluorescent protein, etc.); and the like. The methylated Tat polypeptide can also be detectably labeled, e.g., with a radiolabel. In some embodiments, the methylated Tat polypeptide is biotinylated.

In addition to a methylated Tat polypeptide, an LSD1 polypeptide and a test agent, a variety of other reagents may be included in the screening assay. Suitable additional reagents include reagents such as salts, neutral proteins, e.g. albumin, detergents, etc., including agents that are used to facilitate optimal enzyme activity and/or reduce non-specific or background activity. Reagents that improve the efficiency of the assay, such as protease inhibitors, anti-microbial agents, etc. may be used. The components of the assay mixture are added in any order that provides for the requisite activity. Incubations are performed at any suitable temperature, typically between 4° C. and 40° C. Incubation periods are selected for optimum activity, but may also be optimized to facilitate rapid high-throughput screening. In some embodiments, between 0.1 hour and 1 hour, between 1 hour and 2 hours, or between 2 hours and 4 hours, will be sufficient.

Assays of the invention include controls, where suitable controls include a sample (e.g., a sample comprising the methylated Tat polypeptide and the LSD1 polypeptide in the absence of the test agent). Generally a plurality of assay mixtures is run in parallel with different agent concentrations to obtain a differential response to the various concentrations. Typically, one of these concentrations serves as a negative control, i.e. at zero concentration or below the level of detection.

The effect, if any, of the test agent on demethylation of the methylated Tat polypeptide by the LSD1 polypeptide can be readily determined by assessing the degree of methylation of the substrate methylated Tat polypeptide. Demethylation of the methylated Tat polypeptide can be determined using, e.g., an antibody specific for Lys-51 methylated Tat polypeptide. De-methylation of the methylated Tat polypeptide can also be determined using matrix-assisted laser desorption/ionization-time of flight (MALDI-TOF).

In some embodiments, the effect of the test agent on methylation of the Tat polypeptide is determined using an antibody specific for a Lys-51 methylated Tat polypeptide. The determination step can be carried out using an immunoprecipitation method; an enzyme-linked immunosorbent assay (ELISA); an immunoblot assay; a radioimmunoassay (RIA); and the like, where an antibody specific for Lys-51 methylated Tat polypeptide is used. In some embodiments, an antibody that specifically recognizes a polypeptide comprising the amino acid sequence SYGRKKRRQR (SEQ ID NO:61), where the lysine corresponding to Lys-51 of the amino acid sequence set forth in SEQ ID NO:5 is not methylated, can be used to assess the amount of Tat polypeptide that is not Lys-51 methylated, e.g., the amount of Tat polypeptide that is not methylated at a lysine corresponding to Lys-51 of the amino acid sequence set forth in SEQ ID NO:1.

In other embodiments, the methyl donor is radioactively labeled, and the determining step comprises detecting the radiolabel in the methylated Tat peptide following reaction with the SET domain-containing polypeptide.

In some embodiments, a subject screening method is a cell-free in vitro screening method. In other embodiments, a subject screening method is a cell-based in vitro screening method.

In carrying out a cell-free in vitro screening method, a methylated Tat polypeptide and an LSD1 polypeptide can be present in a test sample in substantially pure form (e.g., at least 90%, at least 95%, at least 98%, or at least 99%, free of contaminants and macromolecules other than the LSD1 polypeptide and the methylated Tat polypeptide), in cell extracts, or other non-purified form.

In some embodiments, in addition to determining the effect of a test agent on LSD1-mediated demethylation of a methylated Tat polypeptide, a test agent is assessed for any cytotoxic activity it may exhibit toward a living eukaryotic cell, using well-known assays, such as trypan blue dye exclusion, an MTT (3-(4,5-dimethylthiazol-2-yl)-2,5-diphenyl-2H-tetrazolium bromide) assay, and the like. Agents that do not exhibit cytotoxic activity are considered candidate agents.

In some embodiments, a subject screening method is a cell-based in vitro screening method. A cell expressing an LSD1 polypeptide and a Tat polypeptides is contacted with a test agent; and the effect, if any, of the test agent on LSD1-mediated demethylation of the Tat polypeptide is determined. The effect of the agent on LSD1-mediated demethylation of the Tat polypeptide can be determined as described above. For example, for the determining step, a cell extract or cell lysate comprising the Tat polypeptide (which may be a mixture of methylated and unmethylated Tat polypeptides) can be analyzed, or the Tat polypeptides (which may be a mixture of methylated and unmethylated Tat polypeptides) can be isolated, e.g., in substantially pure form, from a cell lysate, then analyzed for methylation.

Cell-based in vitro screening methods can be carried out using any of a variety of cells, e.g., primary cells, immortalized cells, and the like. In some embodiments, the cells are eukaryotic cells. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), Jurkat cells (e.g., ATCC TIB-152), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 dells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like. Derivatives of such cells are also suitable for use. Also suitable for use are human T cell lines with latent immunodeficiency virus, e.g., a cell line as described in U.S. Pat. No. 7,232,685.

In some embodiments, the cell produces an endogenous LSD1 polypeptide. In other embodiments, the cell is genetically modified with a nucleic acid comprising a nucleotide sequence that encodes an enzymatically active LSD1 polypeptide. LSD1-encoding nucleotide sequences that are suitable for use for genetically modifying a cell for use in a subject cell-based in vitro screening method are described above.

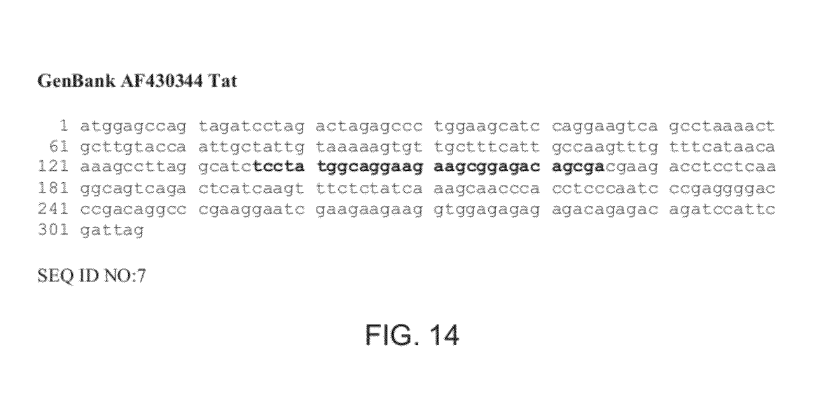

The cell can also be genetically modified with a Tat nucleic acid, e.g., a nucleic acid comprising a nucleotide sequence encoding a Tat polypeptide that comprises at least a lysine corresponding to Lys-51 of the amino acid sequence set forth in SEQ ID NO:5. A suitable Tat nucleic acid comprises a nucleotide sequence having at least about 80%, at least about 85%, at least about 90%, at least about 95%, at least about 98%, at least about 99% or 100% nucleotide sequence identity with a contiguous stretch of from about 30 nucleotides to about 45 nucleotides, from about 45 nucleotides to about 60 nucleotides, from about 60 nucleotides to about 75 nucleotides, from about 75 nucleotides to about 90 nucleotides, from about 90 nucleotides to about 105 nucleotides, from about 105 nucleotides to about 120 nucleotides, from about 120 nucleotides to about 135 nucleotides, from about 135 nucleotides to about 150 nucleotides, from about 150 nucleotides to about 165 nucleotides, from about 165 nucleotides to about 180 nucleotides, from about 180 nucleotides to about 195 nucleotides, from about 195 nucleotides to about 210 nucleotides, from about 210 nucleotides to about 225 nucleotides, from about 225 nucleotides to about 240 nucleotides, from about 240 nucleotides to about 255 nucleotides, from about 255 nucleotides to about 270 nucleotides, from about 270 nucleotides to about 285 nucleotides, or from about 285 nucleotides to about 306 nucleotides of the nucleotide sequence depicted in FIG. 14, where the nucleotide sequence comprises a sequence encoding a lysine corresponding to Lys-51 of the amino acid sequence set forth in SEQ ID NO:5, e.g., where the nucleotide sequence comprises a sequence encoding at least SYGRKKRRQR (SEQ ID NO:61), or a variant thereof.

In some embodiments, a Tat nucleic acid comprises a nucleotide sequence encoding a Tat polypeptide comprising at least SYGRKKRRQR (SEQ ID NO:61), or a variant thereof. In some embodiments, a suitable Tat nucleic acid comprises the nucleotide sequence 5′-TCCTATGGCAGGAAGAAGCGGAGACAGCGA-3′ (SEQ ID NO:62). In some embodiments, the nucleotide sequence encoding the Tat polypeptide is operably linked to a transcriptional control element; and is in some embodiments contained within an expression vector, as described below.

If the genetically modified host cell comprising a Tat nucleic acid does not methylate the encoded Tat polypeptide, the host cell can be genetically modified with a nucleic acid comprising a nucleotide sequence encoding a Set9 polypeptide. Nucleotide sequences encoding Set9 polypeptides are known in the art; see, e.g., GenBank Accession No. BC121055; and FIG. 12B (SEQ ID NO:32) of U.S. Patent Publication No. 2009/0233267. For example, a suitable Set9 nucleic acid comprises a nucleotide sequence having at least about 50%, at least about 60%, at least about 70%, at least about 75%, at least about 80%, at least about 85%, at least about 90%, at least about 95%, at least about 98%, at least about 99%, or 100% nucleotide sequence identity to the nucleotide sequence set forth in FIG. 12B (SEQ ID NO:32) of U.S. Patent Publication No. 2009/0233267.

Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, HIV-based lentivirus vectors, and the like), P1-based artificial chromosomes, yeast plasmids, yeast artificial chromosomes, and any other vectors specific for specific hosts of interest (such as E. coli, mammalian cells, or yeast). Suitable vectors include chromosomal, nonchromosomal and synthetic DNA sequences.

Numerous suitable expression vectors are known to those of skill in the art, and many are commercially available. For example, suitable expression vectors for use in eukaryotic host cells include, but are not limited to, pXT1, pSG5 (Stratagene), pSVK3, pBPV, pMSG, and pSVLSV40 (Pharmacia). However, any other vector may be used so long as it is compatible with the host cell.

Non-limiting examples of suitable eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

Generally, an expression vector will include origins of replication. In addition, the expression vectors include one or more selectable marker genes to provide a phenotypic trait for selection of transformed (genetically modified) host cells such as dihydrofolate reductase or neomycin resistance for eukaryotic cell culture.

Methods of Identifying an Agent that Inhibits LSD1-Mediated Demethylation of a Methylated Tat Polypeptide

The present disclosure provides methods of identifying an agent that inhibits LSD1-mediated demethylation of a methylated Tat polypeptide. The methods generally comprise: a) contacting an LSD1 polypeptide and a methylated Tat polypeptide with a test agent; and b) determining the effect, if any, of the test agent on the methylation of the Tat polypeptide. A test agent that inhibits demethylation of the methylated Tat polypeptide by the LSD1 polypeptide, compared to the level of methylation of the Tat polypeptide in the absence of the test agent, is considered an inhibitor of LSD1-mediated methylation of HIV Tat. In some embodiments, a subject screening method is a cell-free in vitro screening method. In other embodiments, a subject screening method is a cell-based in vitro screening method.

An agent that inhibits demethylation of a methylated Tat polypeptide by an LSD1 polypeptide is considered a candidate agent for inhibiting HIV transcription, and is thus considered a candidate agent for treating an HIV infection in an individual.

A test agent that inhibits demethylation of a methylated Tat polypeptide by an LSD1 polypeptide is a candidate agent for treating an immunodeficiency virus infection. In some embodiments, a test agent of interest inhibits demethylation of a methylated Tat polypeptide by an LSD1 polypeptide by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or more, compared to the degree or level of LSD1-mediated demethylation of the methylated Tat polypeptide in the absence of the test agent.

For example, in some embodiments, a test agent of interest reduces the proportion of the total methylated Tat polypeptides present in the test sample that are demethylated by the LSD1 polypeptide by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more, compared to the proportion of the total methylated Tat polypeptides present in the test sample that are demethylated by the LSD1 polypeptide in the absence of the test agent.

In other words, in some embodiments, a test agent of interest reduces the percentage of demethylated Tat polypeptides (e.g., Tat polypeptides with unmethylated Lys-51 methylated; or Tat polypeptides unmethylated at a position corresponding to Lys-51 of the amino acid sequence set forth in SEQ ID NO:5) in the test sample by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more, compared to the percentage of demethylated Tat polypeptides in the test sample in the absence of the test agent.

In some embodiments, a test agent of interest is one that inhibits Lys-51 demethylation of a methylated Tat polypeptide (e.g., demethylation of Lys-51 or demethylation of a methylated Lys at a position corresponding to Lys-51 of SEQ ID NO:5) by an LSD1 polypeptide with a 50% inhibitory concentration (IC₅₀) of from about 100 μM to about 50 μM, from about 50 μM to about 25 μM, from about 25 μM to about 10 μM, from about 10 μM to about 5 μM, from about 5 to about 1 μM, from about 1 μM to about 500 nM, from about 500 nM to about 400 nM, from about 400 nM to about 300 nM, from about 300 nM to about 250 nM, from about 250 nM to about 200 nM, from about 200 nM to about 150 nM, from about 150 nM to about 100 nM, from about 100 nM to about 50 nM, from about 50 nM to about 30 nM, from about 30 nM to about 25 nM, from about 25 nM to about 20 nM, from about 20 nM to about 15 nM, from about 15 nM to about 10 nM, from about 10 nM to about 5 nM, or less than about 5 nM.

Methods of Identifying an Agent that Increases LSD1 Enzymatic Activity and/or Levels

The present disclosure provides methods of identifying an agent that increases LSD1-mediated demethylation of a methylated Tat polypeptide. The methods generally comprise: a) contacting an LSD1 polypeptide and a methylated Tat polypeptide with a test agent; and b) determining the effect, if any, of the test agent on the methylation of the Tat polypeptide. A test agent that increases LSD1-mediated demethylation of a methylated Tat polypeptide, compared to the level of methylation of the Tat polypeptide in the absence of the test agent, is considered an activator of LSD1-mediated methylation of HIV Tat. In some embodiments, a subject screening method is a cell-free in vitro screening method. In other embodiments, a subject screening method is a cell-based in vitro screening method.

An agent that increases demethylation of a methylated Tat polypeptide by an LSD1 polypeptide is considered a candidate agent for reactivating latent HIV integrated into the genome of an HIV-infected cell. Such an agent could be used in combination therapy with one or more anti-HIV agents.

In some embodiments, a test agent of interest increases demethylation of a methylated Tat polypeptide by an LSD1 polypeptide by at least about 10%, at least about 15%, at least about 20%, at least about 25%, at least about 30%, at least about 35%, at least about 40%, at least about 50%, at least about 60%, at least about 70%, at least about 80%, at least about 90%, at least about 2-fold, at least about 2.5-fold, at least about 3-fold, at least about 5-fold, at least about 10-fold, at least about 25-fold, or more than 25-fold, compared to the degree or level of LSD1-mediated demethylation of the methylated Tat polypeptide in the absence of the test agent.

For example, in some embodiments, a test agent of interest increases the proportion of the total methylated Tat polypeptides present in the test sample that are demethylated by the LSD1 polypeptide by at least about 10%, at least about 15%, at least about 20%, at least about 25%, at least about 30%, at least about 35%, at least about 40%, at least about 50%, at least about 60%, at least about 70%, at least about 80%, at least about 90%, at least about 2-fold, at least about 2.5-fold, at least about 3-fold, at least about 5-fold, at least about 10-fold, at least about 25-fold, or more than 25-fold, compared to the proportion of the total methylated Tat polypeptides present in the test sample that are demethylated by the LSD1 polypeptide in the absence of the test agent.

In other words, in some embodiments, a test agent of interest increases the percentage of demethylated Tat polypeptides (e.g., Tat polypeptides with unmethylated Lys-51 methylated; or Tat polypeptides unmethylated at a position corresponding to Lys-51 of the amino acid sequence set forth in SEQ ID NO:5) in the test sample by at least about 10%, at least about 15%, at least about 20%, at least about 25%, at least about 30%, at least about 35%, at least about 40%, at least about 50%, at least about 60%, at least about 70%, at least about 80%, at least about 90%, at least about 2-fold, at least about 2.5-fold, at least about 3-fold, at least about 5-fold, at least about 10-fold, at least about 25-fold, or more than 25-fold, compared to the percentage of demethylated Tat polypeptides in the test sample in the absence of the test agent.

In some embodiments, a test agent of interest is one that increase Lys-51 demethylation of a methylated Tat polypeptide (e.g., demethylation of Lys-51 or demethylation of a methylated Lys at a position corresponding to Lys-51 of SEQ ID NO:5) by an LSD1 polypeptide with a half-maximal effective concentration (EC₅₀) of from about 100 μM to about 50 μM, from about 50 μM to about 25 μM, from about 25 μM to about 10 μM, from about 10 μM to about 5 μM, from about 5 μM to about 1 from about 1 μM to about 500 nM, from about 500 nM to about 400 nM, from about 400 nM to about 300 nM, from about 300 nM to about 250 nM, from about 250 nM to about 200 nM, from about 200 nM to about 150 nM, from about 150 nM to about 100 nM, from about 100 nM to about 50 nM, from about 50 nM to about 30 nM, from about 30 nM to about 25 nM, from about 25 nM to about 20 nM, from about 20 nM to about 15 nM, from about 15 nM to about 10 nM, from about 10 nM to about 5 nM, or less than about 5 nM.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like. In the context of antibodies, an antibody to a particular antigen can be represented by an alpha (α); for example, an anti-CoREST antibody can be referred to as “α-CoREST”; an anti-FLAG antibody can be referred to as “α-FLAG”; an anti-LSD1 antibody can be referred to as “α-LSD1”; etc.

### Example 1

### Materials and Methods

### Cells, Reagents and Antibodies

HeLa and 293T cells (obtained from the American Type Culture Collection), and the Jurkat clone A2 (Jordan et al. (2003) Embo J 22: 1868-1877) were maintained under standard cell-culture conditions. The following antibodies were commercially available: α-LSD1/KDM1 (#ab51877, abcam, Cambridge, Mass.), α-CoREST (#ab24166, abeam), α-FLAG M2 (#F-3165 Sigma-Aldrich, St. Louis Mo.), α-histone H3K4me2 (#07-030, Millipore, Billerica, Mass.), α-histone H3 (#07-690, Millipore), α-tubulin (#T6074, Sigma-Aldrich), α-Tat (MMS-116P, Covance, Emeryville, Calif.) and α-CD28 (#16-0289-85 eBioscience, San Diego Calif.). α-K51 mono-methylated Tat polyclonal antibodies were previously described. Pagans et al. (2010) Cell Host Microbe 7:234-244. α-CD3 (OKT-3) was obtained from UCSF monoclonal antibodies core facility.

Phenelzine Sulfate was purchased from Spectrum Chemical MHG Corp. (#3032 Gardena, Calif.) and Enzo Life Sciences (#EI-217, Plymouth meeting, PA). Saquinavir was obtained from AIDS Research and Reference Reagent Program, Division of AIDS, NIAID, NIH. Recombinant Human tumor necrosis factor-α (TNF-α) was purchased from Humanzyme (#HZ-1014, Chicago, Ill.).

The synthetic Tat proteins (aa 1-72) was synthesized as previously described (Pagans et al. (2005) PLoS Biol 3: e41) and was synthesized together with the ARM region short peptide (aa 45-58) (Peptide Specialty Laboratories GmbH, Heidelberg, Germany).

The HIV LTR luciferase construct, the EF-1α-Tat/FLAG expression vector, the K51A mutated EF-1α-Tat/FLAG expression vector and the pEF-1α-RL (Renilla luciferase) were described before. Pagans et al. (2010) supra. The FLAG amino acid sequence is DYKDDDDK (SEQ ID NO:59). His-tagged LSD1 prokaryotic expression vector was kindly provided by Dr. Yang Shi (Department of Pathology, Harvard Medical School; Shi et al. (2004) Cell 119: 941-95). Luciferase containing HIV-1 NL4-3 clone—which is capable of multiple rounds of infection and producing luciferase driven from the long terminal repeat (LTR) promoter, was kindly provided by Dr. Warner C Greene (Gladstone Institute of Virology and Immunology).

A modified version of the pSicoR lentiviral vector that encodes the mCherry reporter gene driven by an EF-1α promoter (pSicoRMS) (Grskovic et al. (2007) PLoS Genet. 3: e145; Ventura et al. (2004) Proc Natl Acad Sci USA 101: 10380-10385) was kindly provided by Matthew Spindler (Gladstone Institute of Cardiovascular Disease). Small hairpin RNAs (shRNAs) targeting LSD1 (LSD1 #1:GAAGGCTCTTCTAGCAATA; SEQ ID NO:36; LSD1 #2: CATGTGCCTGTTTCTGCCATG; SEQ ID NO:63) were cloned into pSicoRMS. The pSicoRMS containing a non-targeting control sequence (shScramble: GTCAAGTCTCACTTGCGTC; SEQ ID NO:64) (Hellman et al. (2008) J Biol Chem 283: 4272-4282 and targeting luciferase (shLuciferase: CTTACGCTGAGTACTTCGA; SEQ ID NO:65) was kindly provided by Dr. Silke Wissing (Gladstone Institute of Virology and Immunology). ShRNAs against CoREST and control empty pLKO.1 vector were purchased from Thermo Fischer Scientific (Waltham Mass.).

### In-Gel Digestion and MALDI-TOF Mass Spectrometric Analysis of Tat Protein

C-terminal FLAG tagged Tat protein (Tat/FLAG) purified from Jurkat A2 cells (ca. 100 ng) was further purified by sodium dodecyl sulfate-polyacrylamide gel electrophoresis (SDS-PAGE) (Flamingo gel stain, Bio-Rad Hercules, Calif.). Tat band was excised and washed with 200 μL of 50 mM ammonium bicarbonate containing 50% (v/v) ethanol followed by 200 μL of ethanol for two times. Then, Tat protein in the gel was reduced with 10 mM dithiothreitol (DTT) in 50 mM ammonium bicarbonate for 1 hour at 56° C. and alkylated with 55 mM iodoacetamide in 50 mM ammonium bicarbonate for 30 min at room temperature. After reduction and alkylation, the gel was dehydrated with acetonitrile for 3 times. The gel was rehydrated by adding 200 μL of 50 mM ammonium bicarbonate with 5 ng/μL of chymotrypsin (Roche Applied Science, Penzberg, Upper Bavaria, Germany) and incubated at 30° C. for 2 hours. Peptides digested from Tat protein were extracted from the gel with 1% (v/v) formic acid containing 30% (v/v) acetonitrile followed by 1% (v/v) formic acid containing 60% (v/v) acetonitrile. The extracted peptide solution was dried by speed vac. Then, residual peptides were reconstituted with 30 μL of 0.1% (v/v) trifluoroacetic acid (TFA) containing 2% (v/v) acetonitrile and desalted by ZipTip_{c18} (Millipore) according to the manufacturer's description. 2 μL of cleaned peptides solution eluted from ZipTip_{C18} was deposited on the Bruker metallic MALDI target (MTP384 ground steel, Bruker Daltonics, Billerica, Mass.) and mixed with 2 μL of saturated matrix solution (α-cyano-4-hydroxycinnamic acid solution in 33% (v/v) acetonitrile, 0.1% (v/v) TFA). Peptide mixture was allowed to dry at room temperature. The peptide mixture was analyzed by ultraflex III TOF/TOF (Bruker Daltonics) MALDI-TOF/TOF mass spectrometer, operated in reflector mode for positive ion detection, and controlled by flexControl 3.0 software. For MS/MS acquisitions, the ions of interest were fragmented by laser induced decay; and mass of fragments were analyzed using LIFT mode. Monoisotopic mass was determined using flexAnalysis 3.0 software with the SNAP peak picking algorithm. The modifications of peptides were analyzed using UniMod database in the Biotools software.

### Generation of Bi-Modified Tat Antibodies

The strategy to generate bi-modified Tat (AcK50/Me1K51) specific antibodies was performed as same as previously described. Pagans et al. (2010) supra; Pagans et al. (2010) Methods 53:91. Briefly, keyhole limpet hemocyanin (KLH) conjugated bi-modified arginine-rich motif (ARM) peptides (AcK50/Me1K51) were immunized into rabbits. The amino acid sequence of the peptides was: RKKRRQRRR (SEQ ID NO:52), RK^{Ac}K^{Me}RRQRRR (SEQ ID NO:66), RK^{Ac}KRRQRRR (SEQ ID NO:67), and RKK^{Me}RRQRRR (SEQ ID NO:35), where K^{Ac} is acetylated lysine, and where K^{Me} is monomethylated lysine. The same peptides were used for affinity purification of the anti-serum. Specificity of established antibodies was monitored by Dot-Blot analysis using ARM peptides (unmodified, K50 acetylated, K51 monomethylated and bi-modified) and western blot analysis using synthetic full-length Tat (un-modified, K50 acetylated, K51 methylated and bi-modified).

### In Vitro Enzymatic Assays

Protein expression and purification of recombinant LSD1 and in vitro demethylation reactions of LSD1 (0.5-2.0 μg) with synthetic Tat proteins (3 μg) or purified total cellular histones (8 μg) were performed as previously described. Shi et al. (2004) supra. The reactions were analyzed by western blot using α-Me1K51 Tat antibodies (1 μg/ml). In vitro methylation reactions with synthetic Tat protein (amino acids (aa) 1-72; 1 μg) and ARM peptides (aa 45-58; 100 μM), Set719-KMT7 enzyme (2 μg Millipore), and ³H—S-Adenosyl Methionine (Perkin Elmer) were performed as previously described. Pagans et al. (2010) supra. In vitro acetylation reactions with synthetic Tat proteins (1 μg), ARM peptides (100 μM), glutathione-5-transferase (GST)-p300 histone acetyltransferase (HAT) enzyme (aa 1195-1810; 5 μg; Gu and Roeder (1997) Cell 90: 595-606) and ¹⁴C-acetyl CoA (0.1 μCi; Perkin Elmer) were performed as described. Ott et al. (1999) Curr Biol 9: 1489-1492. Reactions were separated by SDS-PAGE or Tris-Tricine gel electrophoresis and visualized by autoradiography.

### RNAi Experiments

Short interfering RNA (siRNA) analysis for HeLa cells were performed as described. Pagans et al. (2010) supra. Briefly, HeLa cells were transfected with pooled LSD1 and control siRNAs (200 μmol, Dharmacon; Lafayette, Colo.) using Oligofectamine (#58303, Invitrogen, Carlsbad, Calif.) and were retransfected after 48 hr with the HIV LTR luciferase construct (200 ng), Tat-expressing vectors (2 ng), and corresponding amounts of the empty vector by Lipofectamine reagent (#50470, Invitrogen). Cells were harvested 24 hour later and processed for luciferase assays (Luciferase Assay System, #E1501, Promega, Madison, Wis.) or western blotting.

Jurkat A2 cells were transduced with pseudotyped pSicoRMS-derived lentiviral vectors expressing shRNAs against LSD1 (shLSD1#1 and #2), against luciferase (shLuciferase) or a nontargeting shRNA control (ShScramble). These lentiviral vectors also express the mCherry protein under the control of the EF-1α promoter (see under Cells, Reagents, Antibodies). 5 to 10 days after infection, cells were treated with 0.08 ng/ml of TNF-α for 12 hours. Expression of green fluorescent protein (GFP) and mCherry was analyzed by flow cytometory (BD LSRII, Beckton Dickinson, Franklin Lakes, N.J.). The same experiments were performed using pLKO.1-derived vectors expressing shRNAs against CoREST (shCoREST #1 and #2) and empty vector (shControl). In the case of pLKO.1 vector, puromycin was added for selection one day after shRNA infection (1 ng/ml final concentration).

### Chromatin Immunoprecipitation Experiments

Chromatin immunoprecipitations from Jurkat A2 cells were performed as described previously. Kauder et al. (2009) PLoS Pathog 5: e1000495; Pagans et al. (2010) supra. Chromatin solutions were isolated from A2 cells treated with TNF-α (2 ng/ml) and were immunoprecipitated with α-LSD1 antibodies (abeam), α-CoREST antibodies (abcam) or control rabbit pre-immune serum. The immunoprecipitated material was quantified by real-time polymerase chain reaction (PCR) with primers specific for the HIV LTR using the ABI7700 Sequence Detection System (Applied Biosystems, Foster City, Calif.) and the 2× Hot Sybr real-time PCR kit (#HSM-400, McLab, South San Francisco, Calif.). Primer sequences were: HIV LTR upstream: GAGCCCTCAGATCCTGCATA (SEQ ID NO:68), HIV LTR downstream: AGCTCCTCTGGTTTVCCTTT (SEQ ID NO:69).

### Co-Immunoprecipitation Experiments

293T cells were transfected with Tat expressing vector using Lipofectamine reagent (Invitrogen). 2 days after transfection, cells were lysed in immunoprecipitation (IP) buffer (250 mM NaCl, 0.1% NP40, 20 mM NaH₂PO₄ (pH7.5), 5 mM EDTA, 30 mM sodium pyrophosphate, 10 mM NaF and protease inhibitors) and immunoprecipitated with α-FLAG M2 agarose (Sigma-Aldrich) over night at 4° C. Beads were extensively washed and analyzed by western blotting with α-LSD1, α-CoREST or monoclonal α-FLAG antibodies.

### Primary T Cell Model of HIV Latency

The primary T cell HIV latency model was modified from previously reported model. Swiggard et al. (2005) J Virol 79: 14179-14188. The luciferase containing HIV-1 NL4-3 clone, which is capable of multiple rounds of infection and producing luciferase driven from the LTR promoter, was used. NL4-3 Luciferase vector was transfected into 293T cells. Two days after transfection, the transfected supernatants were collected and concentrated by ultra-centrifuge (20,000 rpm 2 hour) and virus concentration was determined by analyzing concentration of p24^{gag} (HIV-1 antigen p24 enzyme-linked immunosorbent assay (ELISA) kit #NEK050A001KT, Perkin Elmer). CD4+ T cells were isolated from human whole blood buffy coats obtained from anonymous donors by centrifugation onto a Histopaque-1077 (#10771, Sigma-Aldrich) cushion, enrichment of T cells by rosetting with sheep red blood cells (#CS115 Colorado Serum, Denver, Colo.), and depletion of non-CD4+ T cells with the CD4+ T cell isolation kit (#130-091-155, Miltenyi Biotec Bergisch Gladbach, Germany) and AutoMACS cell separator (Miltenyi Biotec). Purity of isolated CD4+T cells was confirmed by flow cytometry. Typically, 1 μg of p24^{gag} was used for 5×10⁶ CD4 T cells. The mixture of virus and cells were centrifuged at 2400 rpm for 2 h. After spinoculation, cells were cultured in the presence of 5 μM Saquinavir (NIH) for 3 days and then infected CD4 T cells were stimulated with α-CD3 (2.5 μg/ml, coated) and α-CD28 (1 μg/ml, soluble) in the presence or absence of phenelzine (100 μM-1 mM). After over night incubation, cells were harvested and processed for luciferase assays (Luciferase Assay System, Promega). Cell viability was also determined by propidium iodide staining (#P-3566, Invitrogen).

### Results

### Acetylation of K50 Inhibits Monomethylation of K51 in Tat In Vitro

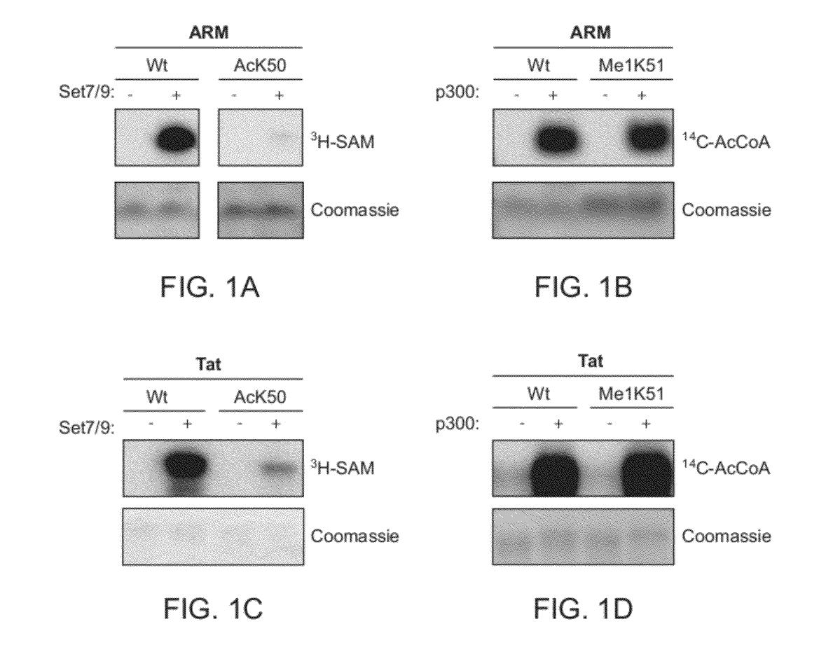

To examine how acetylation of K50 affects monomethylation of the neighboring K51 residue, short synthetic Tat peptides (aa 48-58) carrying an acetylated lysine at position 50 were incubated with recombinant Set7/9/KMT7 enzyme and radiolabeled S-adenosyl-L-methionine (SAM). Reactions were dissolved on a high percentage Tris-Tricine gel and examined by autoradiography. Acetylation at K50 suppressed methylation of the peptide by Set7/9/KMT7 (FIG. 1A). The inverse experiment was also performed. A Tat peptide carrying a monomethyl group at position 51 was incubated with the K50 acetyltransferase p300/KAT3B; no change was observed in acetylation of K50 as compared to an unmodified peptide (FIG. 1B). Similar results were observed when the reactions were performed with full-length synthetic Tat proteins (aa 1-72) carrying acetylated K50 or monomethylated K51 residues (FIGS. 1C and D). These results support previous data that point to a role of K51 monomethylation early in the Tat transactivation cycle (Pagans et al. (2010) supra) and suggest that sequential monomethylation/acetylation could occur within Tat.

FIGS. 1A-D. In vitro acetylation and methylation assays using synthetic Tat peptides (A) In vitro methylation assays of short Tat peptides (aa 45-58). Unmodified or K50-acetylated peptides were incubated with recombinant SET7/9/KMT7 and ³H-radiolabeled S-adenosyl-L-methionine (SAM). Peptides were separated by Tris-Tricine gel electrophoresis and visualized by autoradiography. (B) In vitro acetylation of short Tat peptides. Unmodified or K51-methylated peptides were incubated with recombinant p300-HAT and ¹⁴C-acetyl coenzyme. Peptides were processed as in A. (C) Radioactive in vitro methylation assays of synthetic Tat (aa 1-72) and K50-acetylated synthetic Tat as in A. Reaction products were separated by SDS-PAGE and visualized by autoradiography. (D) Radioactive in vitro acetylation assays of synthetic Tat (aa 1-72) and K51-methylated synthetic Tat as in B. Reaction products were separated by SDS-PAGE and visualized by autoradiography.

### Mass Spectrometry of Immunoprecipitated Tat

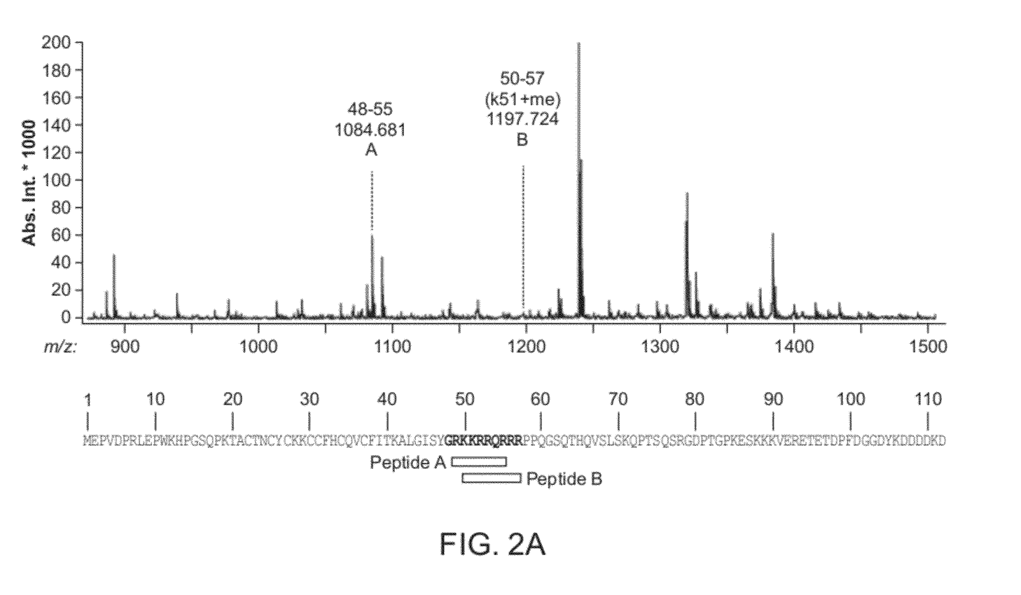

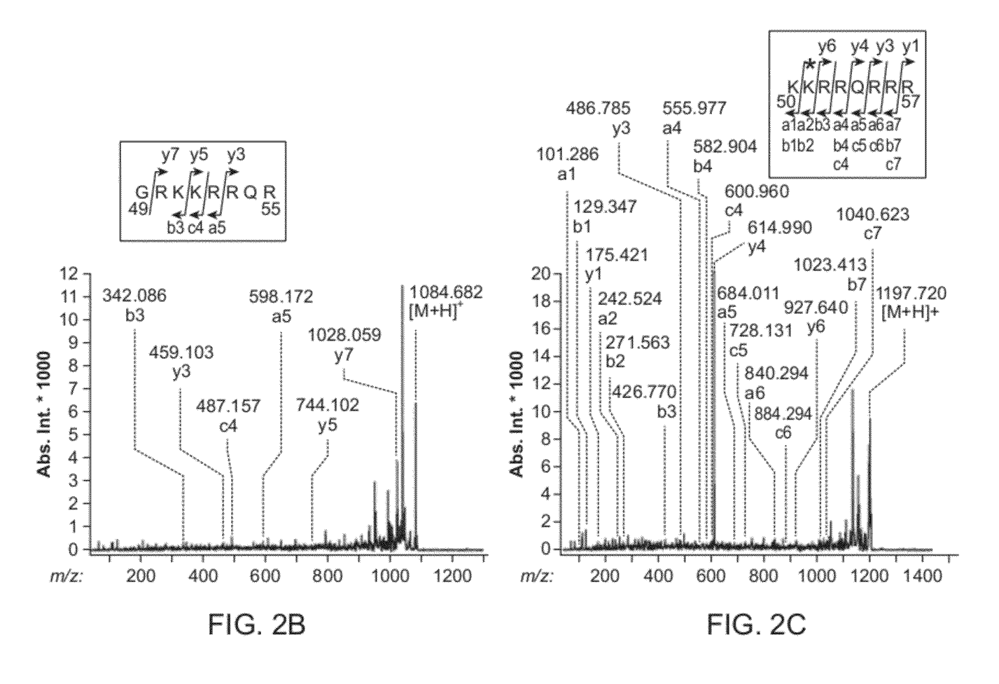

To examine whether methylated/acetylated Tat species is present in cells, mass spectrometry of Tat immunoprecipitated from TNF-α-activated J-Lat A2 cells was performed. This Jurkat-derived cell line harbors an integrated bicistronic lentiviral vector, which expresses FLAG-tagged Tat and GFP from the integrated HIV LTR (LTR-Tat-IRES-GFP) upon stimulation with TNF-α. Jordan et al. (2003) supra. Immunoprecipitated material was separated by SDS-PAGE and stained with FLAMINGO fluorescence dye. The Tat band was cut from the gel and applied to in-gel digestion with chymotrypsin. Residual digested peptides were analyzed by MALDI-TOF/TOF mass spectrometry. A representative matrix-assisted laser desorption/ionization-time of flight (MALDI-TOF) mass spectrometry (MS) spectrum of the digested peptides is shown in FIG. 2A, and more than 100 peptide ion signals were detected. A peptide encompassing the Tat ARM region without modification was detected at 1084.681 m/z, which was identified as the peptide from glycine 48 to arginine 55 in the Tat-FLAG molecule by MALDI-TOF/TOF MS/MS analysis (tandem MALDI-TOF MS analysis) (FIG. 2B). A mass signal was also detected at 1197.724 m/z, which corresponded to the Tat peptide from lysine 50 to arginine 57 carrying a monomethyl group at lysine 51 (FIG. 2C). Bi-modified Tat (AcK50/Me1K51) was not detected in this experiment. In addition, dimethylation was not detected at K51; however, a peptide was detected in which both K50 and K51 carried a mass addition of 42 Da, indicating that these residues are either acetylated or trimethylated in cells.

FIGS. 2A-D. MALDI-TOF mass spectrometric analysis of cellular Tat confirms K51 monomethylation (A) MALDI-TOF MS spectrum of digested peptides from Tat-FLAG (900-1,500 m/z). The peptide ions (designated as A and B) further analyzed by MS/MS are indicated by m/z values and number of amino acid sequence. Positions of peptide A (1084.681 m/z) and peptide B (1197.724 m/z) in the Tat-FLAG molecule are presented in the lower box of the figure. Identified ARM peptides are shorter than anticipated based on the predicted size of chymotrypsin-digested peptides in Tat which is due to a contamination of trypsin-like activity in the chymotrypsin preparations used in this experiment. (B) MALDI-TOF/TOF MS/MS spectra of peptide A ion (1084.681 m/z). (C) MALDI-TOF/TOF MS/MS spectra of peptide B ion (1197.724 m/z). In these spectra, identified fragment ions were denoted by the ion types, a, b, c, y according to the nomenclature by Roepstorff and Fohlman. Roepstorff P, Fohlman J (1984) Biomed Mass Spectrom 11: 601. The assignment of fragment ions to the amino acid sequence of the ARM region of the Tat molecule was inserted in each spectrum. The symbol * indicates methylated amino acid residue.

### Bi-Modified Tat is not Detected in Cells

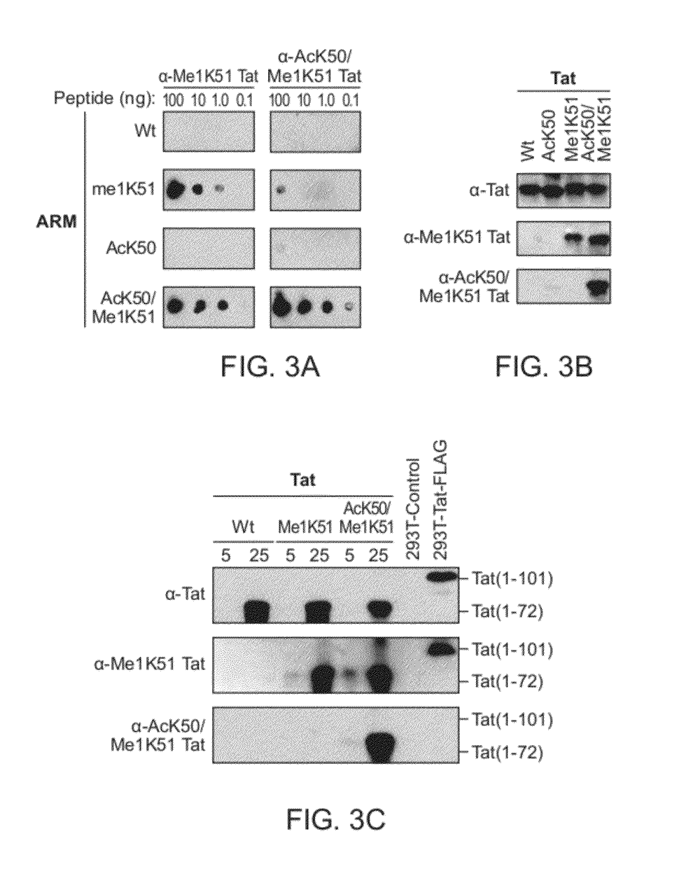

To independently analyze the existence of bi-modified (AcK50/Me1K51) Tat in cells, a polyclonal antiserum specific for doubly modified Tat was generated. ARM peptides carrying an acetyl group at position 50 and a monomethyl group at position 51 were injected into rabbits and affinity purified on a column carrying the bi-modified antigen. The resulting antibodies (α-AcK50/Me1K51 Tat) were specific for the bi-modified ARM peptides and did not react with singly modified peptides in dot blot analysis (FIG. 3A). In contrast, an antiserum that was previously generated against monomethylated K51 in Tat (α-Me1K51 Tat) (Pagans et al. (2010) supra) reacted with ARM peptides monomethylated at K51, as expected, but also showed cross-reactivity with bi-modified peptides (FIG. 3A). The same results were obtained when the antibodies were tested by western blot analysis of full-length synthetic Tat proteins, which carried either one or both modifications. The α-AcK50/Me1K51 Tat antibodies specifically recognized doubly modified Tat while the α-Me1K51 Tat recognized both methylated and doubly modified Tat (FIG. 3B). No cross-reactivity was observed with unmodified or acetylated Tat.

To test whether doubly modified Tat exists in cells, a construct encoding FLAG-tagged Tat was transfected into 293 T cells, and Tat was purified from the cells with α-FLAG agarose. K51 methylated Tat was readily detected by western blot analysis using α-Me1K51 Tat antibodies while no signal was detected with the α-AcK50/Me1K51 Tat antibodies (FIG. 3C). Of note, both antibodies recognized their cognate antigens with similar sensitivities as shown by western blot analysis of full-length synthetic methylated and acetylated/methylated Tat proteins (FIG. 3C). Similar experiments were performed with antibodies against AcK50Me2K51 and AcK50Me3K51 in Tat and showed no reactivity with Tat in cells. This result confirms the data obtained by mass spectrometry, which indicate that doubly-modified Tat is not a major Tat species in cells.

FIGS. 3A and 3B. No detection of K50Ac/K51Me Tat by newly generated Tat antibodies. (A) Dot blot analysis of ARM peptides using α-Me1K51 or α-AcK50/Me1K51 Tat antibodies. (B) Western blot analysis of synthetic Tat (aa 1-72) with α-Tat, α-Me1K51 Tat or α-AcK50/Me1K51 Tat antibodies. (C) Whole cell lysates from 293T cell transfected with a FLAG-tagged Tat-encoding construct were subjected to immunoprecipitation with α-FLAG agarose. Purified proteins were analyzed by western blot analysis using α-Tat, α-Me1K51 Tat or α-AcK50/Me1K51 Tat antibodies.

### SD1/KDM1 Demethylates Tat at K51

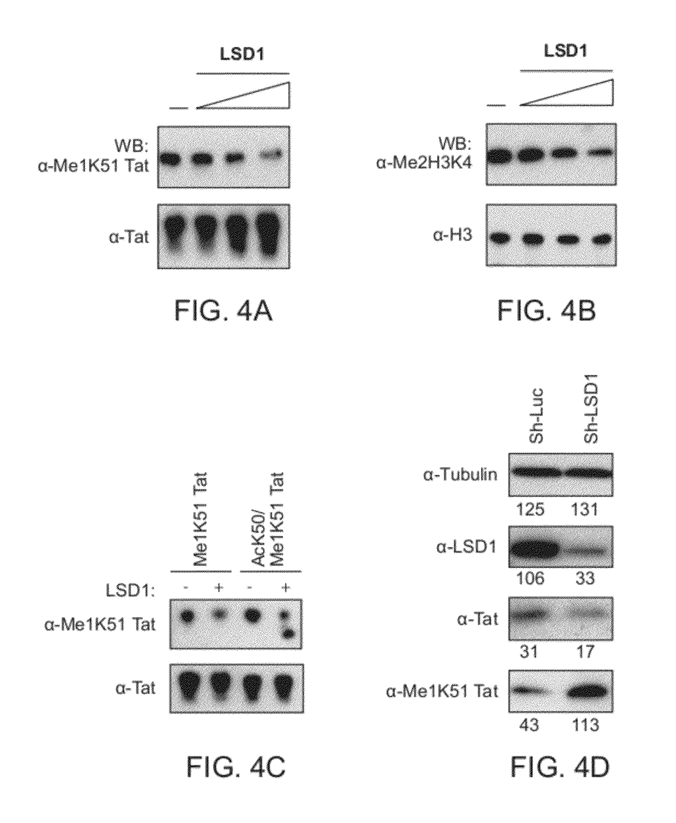

It was speculated that Tat is demethylated at K51 before acetylation occurs. Recombinant LSD1/KDM1 demethylated synthetic monomethylated Tat in a dose-dependent manner as shown by western blot analysis using α-Me1K51 Tat antibodies (FIG. 4A). LSD1/KDM1 also demethylated its cognate substrate, dimethyl lysine 4 in histone H3, as expected (FIG. 4B). Interestingly, LSD1/KDM1 demethylated monomethylated Tat regardless of whether the neighboring K50 residue was acetylated or not, indicating that LSD1/KDM1 could demethylate Tat in cells also immediately after acetylation had occurred (FIG. 4C).

To test whether LSD1/KDM1 is involved in the demethylation of Tat in cells, lentiviral vectors carrying shRNAs against LSD1/KDM1 were introduced into J-Lat A2 cells; endogenous expression of LSD1/KDM1 was reduced (FIG. 4D). Tat expression was induced with TNF-α; and monomethylation of Tat K51 was monitored using western blotting with α-Me1K51 Tat antibodies. Monomethylation of Tat was 2.6-fold enhanced in cells expressing shRNAs against LSD1/KDM1 as compared to cells expressing control shRNAs although the overall expression of Tat was reduced (FIG. 4D). Collectively, these results demonstrate that LSD1/KDM1 demethylates Tat K51 in vitro and in cells.

FIGS. 4A-D. LSD1/KDM1 demethylates mono-methylated K51 in Tat. (A) Synthetic K51-mono-methylated Tat proteins were incubated with increasing amounts of recombinant LSD1/KDM1 (0, 0.5, 1, 2 μg) for 1 hr at 37° C. Reaction products were analyzed by western blotting using α-Tat, α-Me1K51 Tat antibodies. (B) Purified histone proteins were subjected to the same procedure as in (A) as controls for LSD1 activity and analyzed by α-Me2H3K4 or α-histone H3 antibodies. (C) Synthetic K51-mono-methylated Tat or K50-acetylated/K51-mono-methylated Tat proteins were incubated with 1 μg of recombinant LSD1. Both were demethylated by LSD1. (D) Increase in K51-mono-methylation of Tat in LSD1 shRNA-infected J-Lat A2 cells. Whole cell lysates isolated from J-Lat A2 cells infected with shRNAs directed against LSD1 or control shRNAs and stimulated with TNFα were analyzed by western blotting with indicated antibodies.

LSD1/KDM1 Associates with the HIV Promoter In Vivo

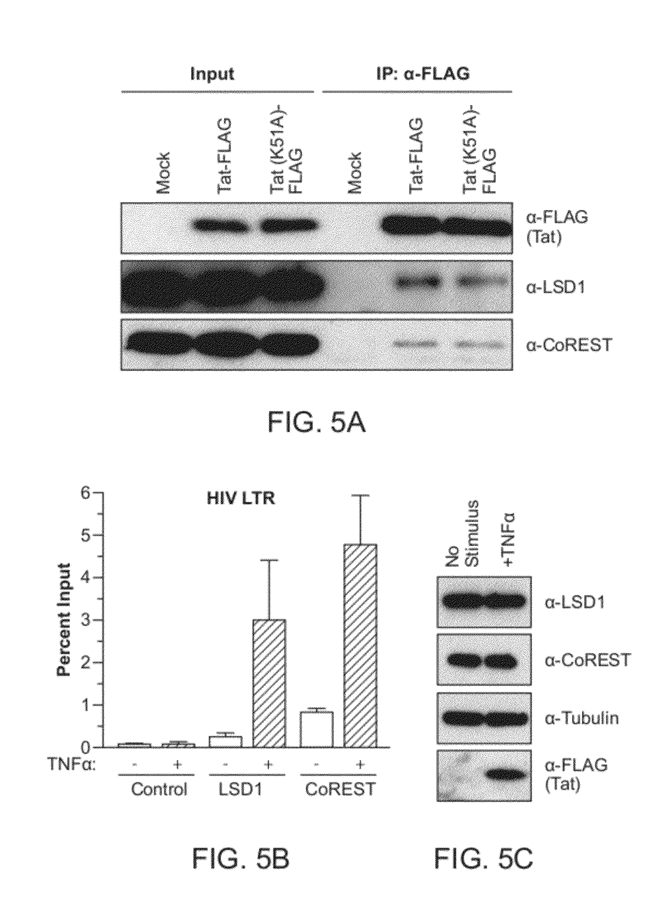

To test whether LSD1/KDM1 interacts with Tat in cells, FLAG-tagged Tat proteins were expressed in 293T cells after transient transfection. Following immunoprecipitation with α-FLAG antibodies, endogenous LSD1/KDM1 was detected by western blotting in the immunoprecipitated material (FIG. 5A). A Tat protein carrying a point mutation in K51 (K51A) also efficiently coimmunoprecipitated with LSD1/KDM1 indicating that the interaction was not dependent on demethylation of K51 in Tat. Similar results were obtained when we tested Tat's interaction with the LSD1/KDM1 cofactor CoREST (Shi et al. (2005) Mol Cell 19: 857-864; 31. Lee et al. (2006) Chem Biol 13: 563-567) suggesting that Tat may recruit a functional LSD1/KDM1/CoREST complex to the HIV promoter.

To test this hypothesis, chromatin immunoprecipitation assays of LSD1/KDM1 and CoREST at the HIV LTR were performed. Chromatin was prepared from J-Lat A2 cells, in which Tat expression was stimulated by TNF-α treatment or which were left nonstimulated. Quantitative PCR analysis of the immunoprecipitated material with primers specific for the HIV LTR indicated that LSD1/KDM1 and CoREST, while only present at low concentrations at the promoter under nonstimulated conditions, were specifically recruited in response to TNF-α stimulation (FIG. 5B). Overall cellular expression of LSD1/KDM1 and CoREST was not upregulated in response to treatment with TNF-α in J-Lat A2 cells (FIG. 5C). These results suggest that the LSD1/KDM1/CoREST complex is recruited to the HIV LTR in response to Tat. However, recruitment may also occur indirectly via other LTR activators in response to TNF-α treatment.

FIGS. 5A-D. In vivo recruitment of LSD1 and CoREST to the HIV LTR. (A) Co-immunoprecipitation of endogenous LSD1 and CoREST with Tat/FLAG and the Tat K51A mutant in transiently transfected 293T cells. (B) Chromatin immunoprecipitation analysis of LSD1 and CoREST in J-Lat A2 cell. A2 cell were stimulated with TNFα overnight and chromatin immunoprecipitation was performed using α-LSD1, α-CoREST or antibodies followed by real-time reverse transcription-polymerase chain reaction (RT-PCR) with primers specific for the HIV LTR region. (C) Whole cell lysates isolated from A2 cell stimulated with TNFα overnight and were analyzed by western blotting using α-LSD1, α-tubulin or α-FLAG antibodies.

### LSD1/KDM1 Acts as an Activator of HIV Transcription

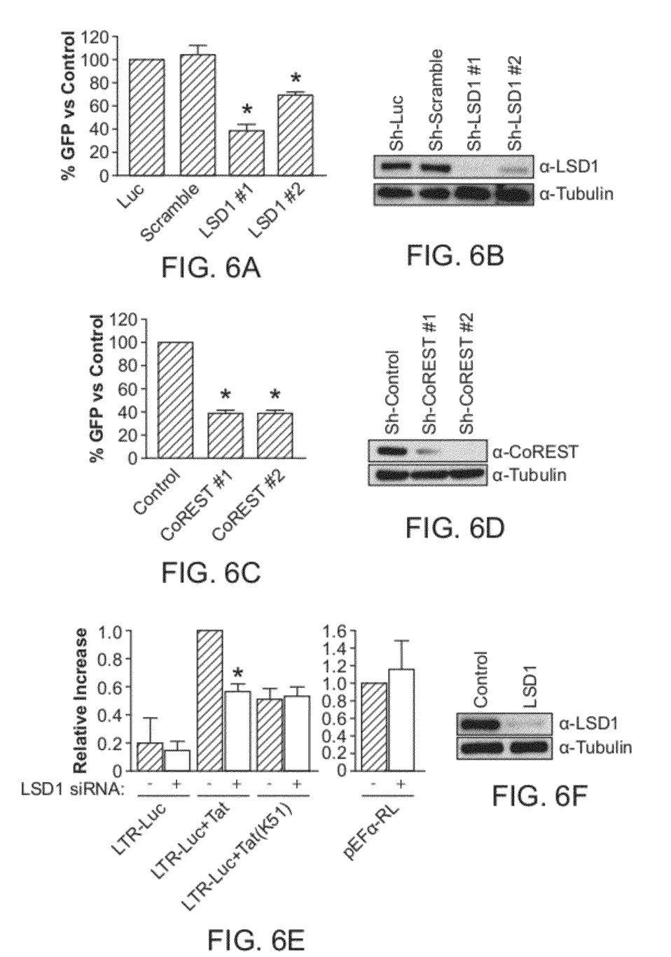

To test the function of LSD1/KDM1 in HIV transcription, A2 cells were transduced with lentiviral vectors expressing two different shRNAs directed against LSD1/KDM1, or control shRNAs directed against firefly luciferase, or a scrambled shRNA. All vectors also expressed the mCherry marker to track infection efficiencies. More than 90% of cells expressed mCherry after lentiviral vector infection, and no difference in infection efficiencies was observed between the different lentiviral vectors. ShRNA-expressing cells were stimulated with TNFα, and expression of GFP was measured by flow cytometry. GFP expression was reduced by 40-60% in LSD1/KDM1 knockdown cells as compared to cell lines expressing luciferase or scrambled shRNAs (FIG. 6A). Interestingly, shRNA#1 had a stronger suppressive effect than shRNA#2 mirroring the degree of LSD1/KDM1 knockdown in these cells (FIG. 6B). A similar suppression of GFP expression was observed in A2 cell lines, in which the expression of CoREST was suppressed (FIG. 6C,D). Collectively, these results demonstrate that a LSD1/KDM1/CoREST complex, normally a suppressor of cellular gene expression, functions as a co-activator of HIV transcription.

To test whether LSD1/KDM1 activates HIV transcription through Tat demethylation, siRNAs specific for LSD1/KDM1, or control siRNAs, were introduced into HeLa cells. Cells were then co-transfected with the HIV LTR luciferase reporter gene and an expression construct for Tat. Tat transactivation of the HIV LTR was suppressed by ˜50% when expression of LSD1/KDM1 was reduced in cells indicating that LSD1/KDM1 is a positive cofactor of Tat transactivation (FIG. 6E). Co-expression of the TatK51A mutant reduced Tat transactivation by ˜50% as previously reported (Pagans et al. (2010) supra) but no further reduction was observed in LSD1/KDM1 knockdown cells, supporting the model that LSD1/KDM1 activates Tat transactivation through K51 demethylation (FIG. 6E). The transcriptional activity of the HIV LTR alone was also reduced in LSD1/KDM1 knockdown cells although values did not reach statistical significance indicating that an additional target for LSD1/KDM1 may exist at the HIV LTR in the absence of Tat (FIG. 6D). Importantly, LSD1/KDM1 knockdown had no suppressive effect on the EF1α promoter that was driving Tat expression in these co-transfection experiments excluding the possibility that LSD1/KDM1 controls Tat expression and not Tat function. Successful knockdown of LSD1/KDM1 expression was confirmed by western blotting (FIG. 6F).

FIGS. 6A-F. A LSD1/KDM1/CoREST complex activates HIV transcription. (A) Lentiviral vectors expressing shRNAs against LSD1 (#1 and #2), luciferase or a scrambled shRNA were infected into J-Lat A2 cells for 5-10 days; cells were then stimulated with a low dose of TNFα (0.08 ng/ml) over night. Number of green fluorescent protein (GFP)+ cells was analyzed by flow cytometry and expressed as percent GFP+ cells in Luciferase-shRNA-infected A2 cells. The average (mean±SEM) of three independent experiments is shown. *corresponds to a p value <0.01. (B) Whole cell lysates from shRNA-expressing cells were analyzed by western blotting using α-LSD1 and α-tubulin antibodies. (C) Lentiviral vectors expressing shRNAs against CoREST (#1 and #2) or control shRNAs were infected into J-Lat A2 cells. The same experiment as in (C) was performed. The average (mean±SEM) of three independent experiments is shown. *corresponds to a p value <0.01. (D) Whole cell lysates from shRNA-infected A2 cells were analyzed by western blotting using α-CoREST and α-tubulin antibodies. (E) SiRNA-transfected HeLa cells (48 h after siRNA transfection) were re-transfected with an HIV LTR luciferase reporter construct and expression vectors for wildtype or K51A mutant Tat. In parallel, EF-1α RL reporter construct was re-transfected. Luciferase or Renilla activities were measured 24 h after plasmid transfections. The relative differences in luciferase activity as compared to wildtype Tat transactivation were calculated. The average (mean±SEM) of three independent experiments is shown. *corresponds to a p value <0.01. (F) Whole cell lysates from siRNA-transfected HeLa cells were analyzed by western blot using α-LSD1 and α-tubulin antibodies.

Phenelzine Suppresses Reactivation of HIV Gene Expression from Latency

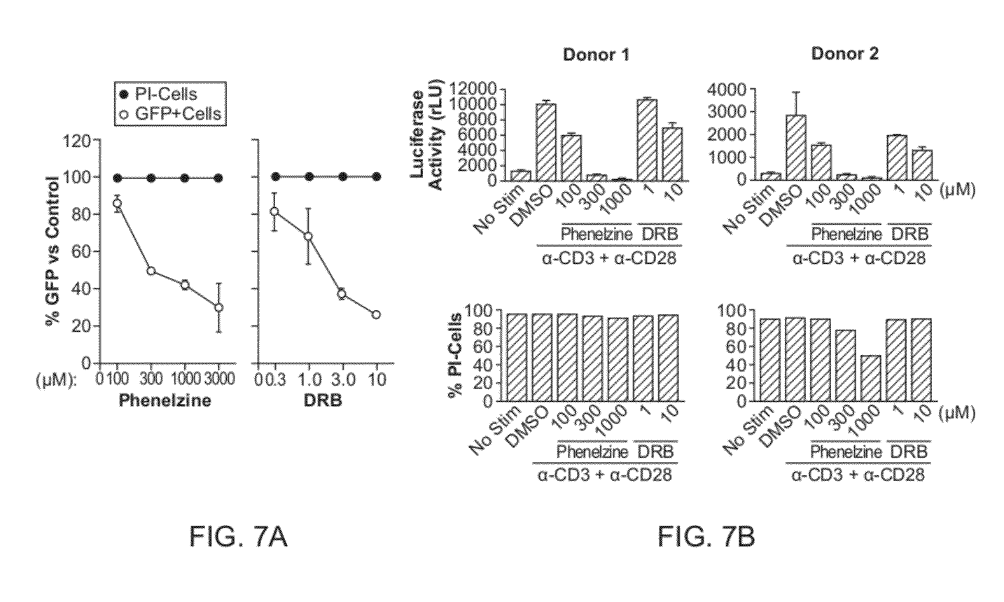

It was recently reported that the monoamine oxidase (MAO) inhibitor phenelzine (phenethylhydrazine) is far more potent in inhibiting LSD1/KDM1 activity in cells than previously appreciated. Culhane et al. (2010) J Am Chem Soc 132: 3164-3176. To test the activity of this agent in HIV infection, J-Lat A2 cells were treated with increasing amounts of phenelzine or the CDK inhibitor 5,6-dichloro-1-β-D-ribofuranosyl-1H-benzimidazole (DRB), a known Tat inhibitor. Phenelzine, similar to DRB, prevented TNFα-mediated activation of gene expression in a dose-dependent manner, albeit at ˜150 fold higher concentrations than DRB (IC₅₀=300 μM FIG. 7A, black circle). No cell toxicity was observed for both agents at the tested concentrations (FIG. 7A, white circle).

The same experiment was performed in a primary T cell model of HIV latency. Quiescent CD4+ T cells were isolated from blood of two healthy donors and were spin-inoculated with an infectious clone of HIV expressing luciferase as previously described. Swiggard et al. (2005) supra. The infected CD4+ cells were cultured with saquinavir for 3 days to ensure that postintegration latency and then treated with increasing amounts of phenelzine followed by stimulation with α-CD3 and α-CD28 antibodies to activate latent HIV transcription. Activation of luciferase expression was successfully suppressed by phenelzine treatment confirming the effectiveness of the drug in the context of a full-length infectious clone of HIV in primary T cells (FIG. 7B). A slight decrease in cell viability was observed in one donor at the highest concentration of phenelzine (FIG. 7B). Interestingly, in primary T cells, phenelzine was more efficient in suppressing HIV gene expression than DRB and also suppressed the basal expression of luciferase in nonactivated cells. Collectively, these results identify LSD1/KDM1 as a potent activator of HIV transcription and potential drug target to prevent reactivation of HIV infection from latency.

FIGS. 7A and 7B. LSD1/KDM1 is a potential drug target in HIV transcription. (A) J-Lat A2 cells were stimulated with 0.08 ng/ml of TNFα in the presence of increasing amounts of phenelzine (100, 300, 1000 or 3000 μM), DRB (0.3, 1, 3 or 10 μM) or dimethylsulfoxide (DMSO) as carrier control overnight. Numbers of GFP+ cells as well as propidium iodide (PI)-negative cells as a measure of intact cell viability were analyzed by flow cytometry. The percentage compared to DMSO-treated control cells was calculated. The average of two independent experiments is shown. (B) Purified resting primary CD4+ T cells were infected at a high multiplicity of infection (M.O.I.) with infectious HIV-NL4-3 luciferase reporter virus. Three days after infection, cells were stimulated with α-CD3 and α-CD28 antibodies in the presence of phenelzine (100, 300, or 1000 μM), DRB (1 or 10 μM) or DMSO overnight followed by analysis for luciferase activity and PI uptake.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of inhibiting transcription of a human immunodeficiency virus (HIV) in a cell infected with the HIV, the method comprising contacting the cell with an agent that inhibits enzymatic activity of a lysine-specific demethylase-1 (LSD1) polypeptide, that reduces the level of the LSD1 polypeptide in the cell, that reduces LSD1-mediated demethylation of a Tat polypeptide in the cell, or that reduces the level or activity of a CoREST polypeptide, wherein said contacting results in inhibition of HIV transcription.

2. The method of claim 1, wherein the agent is a monoamine oxidase inhibitor selected from selegiline, selegiline hydrochloride, dimethylselegilene, brofaromine, phenelzine, tranylcypromine, moclobemide, befloxatone, safinamide, isocarboxazid, nialamide, rasagiline, iproniazide, iproclozide, toloxatone, bifemelane, desoxypeganine, harmine, harmaline, linezolid, and pargyline.

3. (canceled)

4. The method of claim 1, wherein the agent is a polyamine compound of the formula: 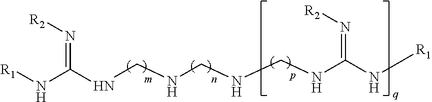
or a salt, solvate, or hydrate thereof, wherein n is an integer from 1 to 12, m and p are independently an integer from 1 to 5, q is 0 or 1, each R₁ is independently selected from the group consisting of C₁-C₈ substituted or unsubstituted alkyl, C₄-C₁₅ substituted or unsubstituted cycloalkyl, C₃-C₁₅ substituted or unsubstituted branched alkyl, C₆-C₂₀ substituted or unsubstituted aryl, C₆-C₂₀ substituted or unsubstituted heteroaryl, C₇-C₂₄ substituted or unsubstituted aralkyl, and C₇-C₂₄ substituted or unsubstituted heteroaralkyl, and each R₂ is independently selected from hydrogen or a C₁-C₈ substituted or unsubstituted alkyl;
or a pharmaceutically acceptable salt thereof.

5. The method of claim 1, wherein the agent is a phenylcyclopropylamine derivative of the formula: 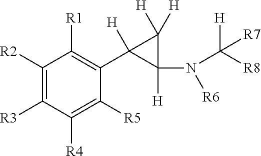
wherein:
each of R1-R5 is independently selected from H, halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanate, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;
R6 is H or alkyl;
R7 is H, alkyl, or cycloalkyl;
R8 is an -L-heterocyclyl wherein the ring or ring system of the -L-heterocyclyl has from 0 to 3 substituents selected from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanate, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido; or
R8 is -L-aryl wherein the ring or ring system of the -L-aryl has from 1 to 3 substituents selected from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanate, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;
where each L is independently selected from —(CH₂)ₙ—(CH₂)ₙ—, —(CH₂)ₙNH(CH₂)ₙ—, —(CH₂)ₙ—O—(CH₂)ₙ—, and —(CH₂)ₙS(CH₂)ₙ—, and where each n is independently chosen from 0, 1, 2, and 3;
or a pharmaceutically acceptable salt thereof.

6. The method of claim 1, wherein the agent is an interfering nucleic acid that specifically reduces the level of LSD1 in the cell or that specifically reduces the level of a CoREST polypeptide in the cell.

7. The method of claim 1, wherein the agent inhibits interaction of an LSD1 polypeptide with a CoREST polypeptide.

8. The method of claim 7, wherein the agent is a CoREST polypeptide that binds LSD1 but does not increase enzymatic activity of LSD1.

9.-10. (canceled)

11. The method of claim 1, wherein the agent is a BHC80 polypeptide.

12. The method of claim 1, wherein the agent is a histone deacetylase-1 or -2 inhibitor.

13. (canceled)

14. A method of treating a human immunodeficiency virus (HIV) infection in an individual having an HIV infection, the method comprising administering to the individual an effective amount of an agent that inhibits enzymatic activity of a lysine-specific demethylase-1 (LSD1) polypeptide, that reduces the level of the LSD1 polypeptide in a cell, or that reduces LSD1-mediated demethylation of a Tat polypeptide in a cell.

15. The method of claim 14, wherein the agent inhibits enzymatic activity of an LSD1 polypeptide, and wherein the agent is a polyamine compound, a phenylcyclopropylamine derivative, or a monoamine oxidase inhibitor.

16.-19. (canceled)

20. The method of claim 14, wherein the agent is an interfering nucleic acid that specifically reduces the level of LSD1 in a cell or that specifically reduces the level of a CoREST polypeptide in a cell.

21. The method of claim 14, wherein the agent inhibits interaction of an LSD1 polypeptide with a CoREST polypeptide, wherein the agent is a CoREST polypeptide that binds LSD1 but does not increase enzymatic activity of LSD1.

22.-24. (canceled)

25. The method of claim 14, wherein the agent is a BHC80 polypeptide.

26. The method of claim 14, wherein the agent is a histone deacetylase-1 or -2 inhibitor.

27.-29. (canceled)

30. A method of treating a human immunodeficiency virus (HIV) infection in an individual, the method comprising:
administering to an individual an effective amount of a first active agent, wherein the first active agent increases lysine-specific demethylase-1 (LSD1) enzymatic activity and/or levels, wherein said first active agent reactivates latent HIV integrated into the genome of a cell in the individual; and
administering to the individual an effective amount of a second active agent, wherein the second active agent inhibits an immunodeficiency virus function selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity.

31. (canceled)

32. An in vitro method for identifying an agent that inhibits transcription of human immunodeficiency virus (HIV), the method comprising:
a) contacting an LSD1 polypeptide and a methylated HIV Tat polypeptide, with a test agent; and
b) determining the effect, if any, of the test agent on the methylation of the Tat polypeptide,
wherein a test agent that inhibits demethylation of the methylated Tat polypeptide by the LSD1 polypeptide, compared to the level of methylation of the Tat polypeptide in the absence of the test agent, is considered a candidate agent for inhibition of HIV transcription.

33. The method of claim 32, wherein the methylated Tat polypeptide comprises a methylated Lys-51 or a methylated lysine at a position corresponding to Lys-51 of the amino acid sequence depicted in FIG. 12 (SEQ ID NO:5), wherein the methylated Lys-51 is monomethylated or dimethylated.

34. (canceled)

35. The method of claim 32, wherein the methylated Tat polypeptide comprises the amino acid sequence SYGRKK^{me}RRQR.

36. (canceled)

37. The method of claim 32, wherein the methylated Tat polypeptide comprises a radiolabelled methyl group.

38.-39. (canceled)
